(19) United States
(12) Patent Application Publication
(10) Pub. No.: US 2026/0256923 A1
(43) Pub. Date: Sep. 3, 2026

(54) NEW RECRUITMENT ELEMENT OF UBIQUITIN LIGASE AND USE THEREOF

(71) Applicant: SHENZHEN LINGGENE BIOTECH CO., LTD., Shenzhen, Guangdong (CN)

(72) Inventors: Yiying LIANG, Shenzhen, Guangdong (CN); Cuihua LIANG, Shenzhen, Guangdong (CN)

(21) Appl. No.: 19/469,326

(22) PCT Filed: Mar. 28, 2024
(86) PCT No.: PCT/CN2024/084481

§ 371 Date: Sep. 25, 2025

(30) Foreign Application Priority Data
Mar. 30, 2023 (CN) 202310361868.3
Nov. 27, 2023 (CN) 202311594108.3

(51) Int. Cl. A61K 47/54; A61P 35/00

(52) U.S. Cl. CPC A61K 47/549; A61K 47/545; A61P 35/00

(57) ABSTRACT

A new recruitment element of a ubiquitin ligase and the use thereof, in particular, the use of GROs as a recruitment element of MDM2 in a drug. By means of recruiting MDM2, the GROs can be used in a constructed proteolysis-targeting agent, comprising a PROTAC drug. The proteolysis-targeting agent comprises GROs, a linker and a target protein ligand, or comprises GROs, a linker and an E3 ubiquitin ligase ligand. In the use in the proteolysis-targeting agent, GROs are used as a new recruitment element of MDM2, and also endow the proteolysis-targeting agent with targeting and penetrative properties for diseased cells (such as tumors), so that the non-specific distribution of the proteolysis-targeting agent in normal tissue is reduced, the toxic side effects of the proteolysis-targeting agent are reduced, the capability and efficiency of the proteolysis-targeting agent for entering diseased cells (such as tumors) is improved. In addition, GROs can be used for treating tumor diseases or other related diseases that require the degradation of a target protein.

### TECHNICAL FIELD

The present disclosure pertains to the technical field of biomedicine, and specifically relates to a novel recruitment element of a ubiquitin ligase and use thereof, in particular, to use of a GRO as a recruitment element of MDM2 in a medicament.

### BACKGROUND

Targeted protein degradation is a brand-new breakthrough drug development strategy that leverages the inherent protein degradation pathway in cells to induce the direct degradation of pathogenic target proteins. The novel pharmaceutical forms include various types, such as a PROTAC, a molecular glue, an LYTAC, an ATAC, an AbTAC, an ATTEC, an AUTAC, and an AUTOTAC. A PROTAC (proteolysis targeting chimera) is a bifunctional molecule consisting of three parts: a target protein ligand, a linker, and an E3 ubiquitin ligase recruitment element. After a PROTAC enters the cell, the target protein ligand in its structure specifically binds to the target protein, and the E3 ligase recruitment element at the other end recruits the E3 ligase to form a ternary complex of target protein-PROTAC-E3. The E3 ubiquitin ligase mediates ubiquitination of the target protein by the ubiquitin-binding enzyme E2, and the polyubiquitinated target protein is then transported to the proteasome for degradation, thereby reducing the level of the target protein. In the process described above, the target protein ligand does not need to occupy the binding site for a long time. Thus, PROTACs may act in multiple cycles within the cell. Based on the special action mechanism of PROTACs, PROTAC drugs can overcome the drug development barriers associated with undruggable targets and difficult-to-drug targets.

MDM2 holds a special position in the development of PROTACs. In one aspect, it serves as one of the four E3 ubiquitin ligases commonly used in PROTACs (the other three are CRBN, VHL, and IAP, respectively) and is recruited by recruitment elements in PROTACs for ubiquitination and degradation of a variety of target proteins and disease treatment. In another aspect, MDM2 is also a protooncogene that is highly expressed in various tumors. As a key negative regulator of tumor suppressor p53, it plays an important role in the development and progression of tumors. Consequently, MDM2 is also often recognized by PROTACs as a target protein for ubiquitination and degradation for tumor treatment.

At present, the recruitment elements of MDM2 are mainly its ligand molecule Nutlin-3a and derivatives thereof.

However, these ligands may produce toxicity during use, which may be due to the activation of p53 in the bone marrow caused by incorrect dosage, leading to blood toxicity, including thrombocytopenia and neutropenia.

Therefore, there is a need to find novel recruitment elements of MDM2 for target protein degradation drugs and PROTAC construction.

In addition, the traditional PROTACs themselves do not have diseased cell selectivity, which will result in their non-specific distribution in normal tissues, leading to systemic toxicity. To achieve the selectivity of PROTACs for diseased cells, the current strategy is to conjugate PROTACs with antibodies, small molecules, polypeptides, oligonucleotides, or nucleic acid aptamers with the targeting function for diseased cells. However, the introduction of these target heads has made the molecular composition of PROTACs more complex, increasing the difficulty levels of both synthesis and mass production. Therefore, it is an important challenge to construct a novel PROTAC that is ingenious and simple and can achieve cell selectivity without the need to add additional target heads that recognize diseased cells.

In the molecular structure of the traditional PROTACs, both the E3 ubiquitin ligase recruitment element and the target protein ligand are mainly small molecules, which facilitates the rapid entry of the PROTACs into the cells to play a role. However, some difficult-to-drug target proteins do not have small molecule ligands. For such targets, attempts have been made to design PROTACs using macromolecular ligands, such as polypeptides, oligonucleotides, nucleic acid aptamers, and monoclonal antibodies. However, macromolecular PROTACs are only at a conceptual stage, and the main reason is that the large molecular weight causes difficulty in cell penetration. Therefore, macromolecular PROTACs still need to be assisted by techniques such as transfection, membrane-penetrating molecules, and microinjection to enter cells, which greatly limits the application of macromolecular PROTACs. Therefore, it is necessary to construct macromolecular PROTACs that can penetrate cells without auxiliary means, so as to avoid the problem that some difficult-to-drug target proteins do not have small molecule ligands and improve the application prospect of macromolecular PROTACs.

Nucleolin (NCL) is a multifunctional shuttle protein that shuttles among the nucleus, cytoplasm, and even the cell membrane of diseased cells, such as tumor cells, immune cells, and vascular endothelial cells. The NCL on the cell membrane surface can also serve as a receptor or co-receptor for a variety of protein molecules, bacteria, and viruses.

Abnormal expression of NCL affects the development and progression of various diseases such as inflammatory responses, viral infections, and tumors. NCL has an increased expression level and increased cell membrane distribution in a variety of diseased cells (such as tumors), affecting multiple processes such as cell proliferation, apoptosis, and metastasis. GROs (guanine-rich oligonucleotides), including AS1411 nucleic acid aptamers, are single-stranded DNA molecules that can specifically bind to NCL. GROs can specifically recognize NCL highly expressed on the membrane surface of diseased cells (such as tumors), penetrate cells by the shuttle effect of NCL, and can even reach the nucleus. AS1411 underwent phase I and phase II clinical trials as an antitumor targeted drug, showing good safety, but it did not advance to phase III clinical trials.

However, currently, GROs, as a novel recruitment element of MDM2, are not used for the development and research of target protein degradation drugs and PROTACs.

### SUMMARY

A first objective of the present disclosure is to provide a novel recruitment element of a ubiquitin ligase and use thereof, in particular, use of a GRO as a recruitment element of MDM2 in a drug. The GRO can recruit MDM2, and MDM2 exerts E3 ubiquitin ligase activity.

Preferably, the drug is a targeted protein degrader (TPD), for example, a targeted protein degrader based on the ubiquitination-proteasome system, endosome-lysosome, or autophagy-lysosome, such as a proteolysis targeting chimera (PROTAC), a molecular glue, a chaperone-mediated protein degrader (CHAMP), a lysosome targeting chimera (LYTAC), a GlucTAC, an antibody-PROTAC conjugate (AbTAC), an AUTAC, an AUTOTAC, an ATTEC, an antibody-PROTAC conjugate, and an autophagy degradation targeting chimera (MADTAC) (see, e.g., Zhou Luozhu, Sheng Chunquan. Advances and Prospects in Targeted Protein Degradation [J]. Journal of Pharmaceutical Practice and Service, 2023, 41(06): 341-351+365; Liu Jinghong, Chen Yimin, Cai Xiaoqing. Research Advances in New Technologies in Targeted Protein Degradation [J]. Acta Pharmaceutica Sinica, 2022, 57(02): 313-320; Paudel R R, Lu D, Roy Chowdhury S, Monroy E Y, Wang J. Targeted Protein Degradation via Lysosomes. Biochemistry. 2023 Feb. 7; 62(3): 564-579, etc.).

Further preferably, the target protein degradation drug is a PROTAC drug.

In the present disclosure, the GRO is a guanine-rich oligonucleotide capable of specifically binding to NCL; preferably, the GRO is a guanine-rich oligonucleotide comprising one or more GGT motifs and having G4 structural characteristics. The GRO exerts a novel function of recruiting MDM2 and endows PROTACs with targeting and penetration effects on diseased cells (such as tumors).

Further preferably, the GRO has stable G4 structural characteristics, wherein G4 structural characteristic signals can be detected using the methods described in the prior art (see, e.g., Yu Yuan, Hu Fang, Xia Yuan, et al. Progress in Detection Methods and Biological Research of G-Quadruplex [J]. Chemistry of Life, 2021, 14(10): 2146-2155; Gao Juan, Yuan Gu, Xu Ming. The Detections, Functions and Regulations of G-Quadruplex [J]. Progress in Physiological Sciences, 2014, 45(5): 364-371), such as probes (e.g., specific antibodies, specific fluorescent ligands, radioactive labels, etc.), circular dichroism (CD) spectroscopy, nuclear magnetic resonance (NMR), ultraviolet spectrometry, molecular fluorescence spectroscopy, or single molecule fluorescence resonance energy transfer (FRET).

In some embodiments of the present disclosure, detection is performed using a fluorescent probe (e.g., N-methyl mesoporphyrin IX (NMM) or O-phenanthroline derivative), and the GRO has a stable G4 structural characteristic signal (see, e.g., Zhang Suge, Sun Hongxia, Tang Yalin. Research Progress in the Probes Targeting DNA G-quadruplex [J]. Chemistry Online, 2016, 79(5): 387-394).

In one embodiment of the present disclosure, when N-methyl mesoporphyrin IX (NMM) is used as a fluorescent probe for detection, AS1411 can form stable G4 structural characteristics in the presence or absence of K⁺. Specifically, the detection method may comprise the following steps: adding a test GRO to a solution with or without K⁺, heating the mixture at 90-99° C. for 5-10 min, incubating the mixture on ice, adding NMM, incubating the mixture in the dark at room temperature, and detecting the fluorescence intensity (e.g., as described in Example 1 of the present disclosure).

Further preferably, the GRO comprises a chemical modification, a nucleic acid unit replacement, or a linkage to a functional group on the GRO; these modifications or functional groups may be used to improve the stability of PROTACs, provide a detection signal, or form a composition with other substances. The chemical modification is that at least one base is modified, and the chemical modification includes at least one of phosphorylation, methylation, amination, sulfydrylation, isotopication, phosphorothioate backbone modification, methoxy modification, and fluoro modification; the nucleic acid unit replacement is that at least one nucleic acid unit is replaced by LNA, UNA, or GNA; the functional group includes at least one of a fluorophore, a radioactive group, a therapeutic drug, biotin, digoxigenin, a nano-luminescent material, a nucleic acid substance, or an enzyme label.

Preferably, the GRO has 4-100 (e.g., 4, 5, 6, 8, 10, 12, 14, 16, 18, 20, 22, 24, 26, 28, 30, 40, 50, 60, 80, or 100) nucleotides.

Preferably, the GRO is one of DNA aptamer AS1411 and an AS1411 derivative/analog.

The AS1411 derivative/analog is a GRO having 60% or more (e.g., 65%, 70%, 75%, 80%, 85%, 90%, 95%, or more) homology to AS1411, comprising one or more GGT motifs, having stable G4 structural characteristics, and being capable of specifically binding to NCL.

Specifically, the AS1411 derivative/analog is one of GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M having the nucleotide sequences set forth in SEQ ID NOs: 2-4 and 20-47, respectively.

Specifically, AS1411 has the sequence set forth in SEQ ID NO: 1: 5′-GGTGGTGGTGGTTGTGGTGGTGGTGG-3′.

In a preferred embodiment of the present disclosure, the use is use of AS1411 as a recruitment element of MDM2 in the preparation of PROTACs.

In other embodiments of the present disclosure, the use is use of an AS1411 derivative/analog (e.g., those described above, particularly GRO29A, GRO15A, or AT11) as a recruitment element of MDM2 in the preparation of PROTACs.

In one embodiment of the present disclosure, the GRO is iSN04 having the sequence as follows: 5′-AGATTAGGGTGAGGGTGA-3′; iSN04 is an optional GRO, but is not a preferred GRO.

A second objective of the present disclosure is to provide use of a GRO as a recruitment element of MDM2 in PROTACs. In the use, the GRO, as a novel recruitment element of MDM2, endows PROTACs with targeting and penetration effects on diseased cells (such as tumors), reduces non-specific distribution of PROTACs in normal tissues, reduces toxic and side effects of PROTACs, and improves the ability and efficiency of PROTACs to enter diseased cells (such as tumors).

In the use, the GRO, as a recruitment element of MDM2, is used in constructing PROTACs. Since GROs can specifically recognize NCL highly expressed on the membrane surface of diseased cells (such as tumors), the PROTACs constructed by using the GRO as a novel recruitment element of MDM2 specifically bind to NCL via the GRO moiety and penetrate the cells by means of the shuttle effect of the NCL, thereby exerting targeting effects on diseased cells (such as tumors) without additionally introducing other target heads that recognize diseased cells (such as tumors). Compared with PROTACs that require additional introduction of other target heads that recognize diseased cells (such as tumors), the PROTACs are more ingenious and simple, which is conducive to synthesis and mass production. Therefore, macromolecular PROTACs can be constructed, so as to avoid the problem that some difficult-to-drug target proteins do not have small molecule ligands and achieve cell penetration without any auxiliary means, thereby improving the application prospect of macromolecular PROTACs.

Based on the two roles that MDM2 plays, either as an E3 ubiquitin ligase or as a target protein recruited by PROTACs, the use of a GRO as a recruitment element of MDM2 in PROTACs has the following two modes:

When MDM2 serves as the E3 ubiquitin ligase recruited by PROTACs, the GRO functions as a recruitment element of MDM2 and is conjugated to a target protein ligand via a linker to degrade the target protein. When MDM2 serves as the target protein recruited by PROTACs, the GRO functions as a recruitment element of MDM2 and is conjugated to an E3 ubiquitin ligase ligand via a linker to degrade MDM2.

Preferably, the target protein ligand includes, but is not limited to, small molecule compounds, oligonucleotides, oligopeptides, polypeptides, proteins, and the like; further preferably, the oligonucleotides include oligonucleotides such as nucleic acid aptamers; the target protein is a protein that needs to be targeted for degradation, including but not limited to, mutant proteins, pseudokinases, transcription factors, scaffold proteins, membrane proteins, and the like.

The ligand of the E3 ubiquitin ligase includes, but is not limited to, small molecule compounds, oligonucleotides, oligopeptides, polypeptides, proteins (e.g., antibodies), and the like; further preferably, the oligonucleotides include oligonucleotides such as nucleic acid aptamers. The E3 ubiquitin ligase includes, but is not limited to, CRBN, VHL, IAP, cbl-b, MDM2, DCAF15, RNF114, DCAF16, KEAP1, FEM1B, and the like.

In a preferred embodiment of the present disclosure, in the use of a GRO as a recruitment element of MDM2 in PROTACs, the GRO can be linked to one or more target protein ligands to construct multifunctional molecule PROTACs and degrade one or more target proteins.

In a preferred embodiment of the present disclosure, in the use of a GRO as a recruitment element of MDM2 in PROTACs, the GRO can be linked to one or more E3 ubiquitin ligase ligands for recruiting one or more E3 ubiquitin ligases to degrade MDM2.

In a preferred embodiment of the present disclosure, an additional ligand, including a target protein ligand, an E3 ubiquitin ligase ligand, or the like, may be linked to any base, sugar, or phosphate backbone at the 3′ end or 5′ end or in the middle of the GRO. In another preferred embodiment of the present disclosure, a modification may be made on or an additional functional group may be linked to any base, sugar, or phosphate backbone at the 3′ end or 5′ end or in the middle of the GRO. These modifications or functional groups may be used to improve the stability of PROTACs, provide a detection signal, or form a composition with other substances. Preferably, the modification that at least one base is modified includes phosphorylation, methylation, amination, sulfydrylation, or isotopication.

Preferably, the functional group includes at least one of a fluorophore, a radioactive group, a therapeutic drug, biotin, digoxigenin, a nano-luminescent material, a nucleic acid substance, or an enzyme label.

A third objective of the present disclosure is to provide a targeted protein degrader comprising a GRO, a linker, and a target protein ligand, or comprising a GRO, a linker, and an E3 ubiquitin ligase ligand, wherein the target protein ligand or the E3 ubiquitin ligase ligand is conjugated to the GRO via the linker to prepare the targeted protein degrader.

In one embodiment of the present disclosure, the targeted protein degrader has the following structure:

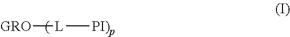

wherein
L is a linker;
PI is a target protein ligand moiety;
p is an integer of 1-100 (e.g., 1, 2, 3, 4, 5, 6, 7, 8, 9, 10, 12, 14, 15, 20, 25, 30, 35, 40, 50, 60, 80, or 100).

In a preferred embodiment of the present disclosure, p is 1, that is, general formula I is GRO-L-PI.

Specifically, the GRO is as described in the first objective of the present disclosure.

Specifically, L may be linked to any base, sugar, or phosphate backbone at the 3′ end or 5′ end or in the middle of the GRO.

In some embodiments of the present disclosure, L is linked to the 3′ end of the GRO.

In some embodiments of the present disclosure, L is linked to the 5′ end of the GRO.

In some embodiments of the present disclosure, the GRO moiety in general formula I has the following structure:

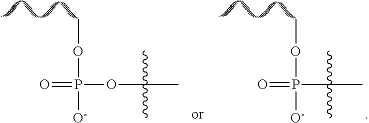

Specifically, L has the following structure:

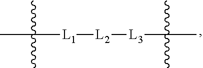

wherein
L₁ is a divalent group linked to the GRO and may be selected from: a single bond, —O—(C₀-C₆ alkylene)-, —S—(C₀-C₆ alkylene)-, —N(R^{L1})—(C₀-C₆ alkylene)-, —N(R^{L2})C(O)—(C₀-C₆ alkylene)-, —OP(O)(OR^{L1})O—(C₀-C₆ alkylene)-, —C(O)—(C₀-C₆ alkylene)-, —C(S)—(C₀-C₆ alkylene)-, and —CON(R^{L1})—(C₀-C₆ alkylene)-;
L₃ is a divalent group linked to PI or E3L and may be selected from: a single bond, —O—(C₀-C₆ alkylene)-, —S—(C₀-C₆ alkylene)-, —C(O)—(C₀-C₆ alkylene)-, —C(S)—(C₀-C₆ alkylene)-, —N(R^{L3})—(C₀-C₆ alkylene)-, —CON(R^{L3})—(C₀-C₆ alkylene)-, —N(R^{L3})CO—(C₀-C₆ alkylene)-, —SO₂—(C₀-C₆ alkylene)-, and —SO—(C₀-C₆ alkylene)-;
L₂ is a single bond or a divalent, saturated or unsaturated, linear or branched C₁-C₅₀ hydrocarbon chain (e.g., C1-C20 alkyl chain), wherein 0-6 methylene units in the hydrocarbon chain (e.g., alkyl chain) are independently substituted with: —CY—, —O—, —S—, —S—S—, —C(O)—, —C(S)—, —C(O)O—, —OC(O)—, —OC(O)O—, —C(O)N(R^{L2})—, —N(R^{L2})C(O)—, —N(R^{L2})C(O)O—, —N(R^{L2})C(O)N(R^{L2})—, —N(R^{L2})—, —S(O)₂—, —S(O)₂N(R^{L2})—, —N(R^{L2})S(O)₂—, —S(O)—, —S(O)N(R^{L2})—, —N(R^{L2})S(O)—, —P(O)(OR^{L2})O—, —P(O)—, —P(O)N(R^{L2})—, —P(O)(N(R^{L2})₂)-, —OP(O)(OR^{L2})₂N(R^{L2})—, —P(O)(OR^{L2})₂N(R^{L2})—, —N(R^{L2})P(O)(OR^{L2})O—, —N(R^{L2})P(O), —Si(R^{L2})₂, —C(═N—CN)—,

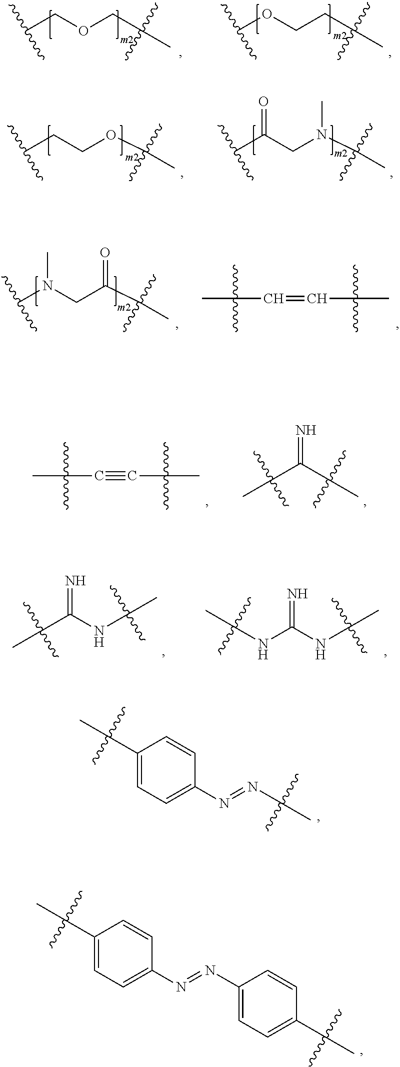

an amino acid residue, a nucleotide residue, an oligonucleotide residue, or an oligopeptide residue, wherein m2 is selected from integers between 1 and 10 (e.g., 1, 2, 3, 4, 5, 6, 7, 8, 9, or 10), and each —CY— is independently an optionally substituted divalent ring selected from: arylene, cycloalkylene, and heterocyclylene; H in the hydrocarbon chain may be optionally substituted with one or more groups selected from: halogen, cyano, nitro, azido, —OR^{L0}, —C(O)R^{L0}, —C(S)R^{L0}, —C(O)OR^{L0}, —C(S)SR^{L0}, —OC(O)R^{L0}, —OC(S)R^{L0}, —OC(S)SR^{L0}, —C(O)N(R^{L0})₂, —OC(O)N(R^{L0})₂, —N(R^{L0})C(O)OR^{L0}, —N(R^{L0})SO₂R^{L0}, —SO₂N(R^{L0})₂, —OSO₂N(R^{L0})₂, —N(R^{L0})C(O)R^{L0}, —N(R^{L0})₂, —SR^{L0}, —SOR^{L0}, —SO₂R^{L0}, —OSO₂R^{L0} C₁-C₁₀ alkyl, C₂-C₁₀ alkenyl, C₂-C₁₀ alkynyl, C₁-C₁₀ haloalkyl, C₁-C₁₀ haloalkoxy, —(C₀-C₆ alkylene)-(C₃-C₁₀ cycloalkyl), —(C₀-C₆ alkylene)-(C₆-C₁₀ aryl), and —(C₀-C₆ alkylene)-(4- to 10-membered heterocyclyl);
R^{L0}, R^{L1}, R^{L2}, and R^{L3} are independently selected from: H, C₁-C₁₀ alkyl, C₂-C₁₀ alkenyl, C₂-C₁₀ alkynyl, —(C₀-C₆ alkylene)-(C₃-C₁₀ cycloalkyl), —(C₀-C₆ alkylene)-(C₆-C₁₀ aryl), and —(C₀-C₆ alkylene)-(4- to 10-membered heterocyclyl), wherein H in the C₁-C₁₀ alkyl, C₂-C₁₀ alkenyl, C₂-C₁₀ alkynyl, C₀-C₆ alkylene, C₃-C₁₀ cycloalkyl, C₆-C₁₀ aryl, or 4- to 10-membered heterocyclyl may be optionally substituted with one or more groups selected from: halogen, cyano, nitro, azido, hydroxy, amino, sulfydryl, carboxyl, C₁-C₁₀ alkyl, C₂-C₁₀ alkenyl, C₂-C₁₀ alkynyl, C₁-C₁₀ haloalkyl, C₁-C₁₀ haloalkoxy, —(C₀-C₆ alkylene)-(C3-C₁₀ cycloalkyl), —(C₀-C₆ alkylene)-(C₆-C₁₀ aryl), and —(C₀-C₆ alkylene)-(4- to 10-membered heterocyclyl).

Specifically, each —CY— is independently selected from the following optionally substituted divalent rings: phenylene, bicyclic arylene, tricyclic arylene, monocyclic cycloalkylene, bicyclic cycloalkylene, tricyclic cycloalkylene, monocyclic heteroarylene, bicyclic heteroarylene, tricyclic heteroarylene, monocyclic heterocycloalkylene, bicyclic heterocycloalkylene, and tricyclic heterocycloalkylene.

In some embodiments of the present disclosure, each —CY— is independently selected from:

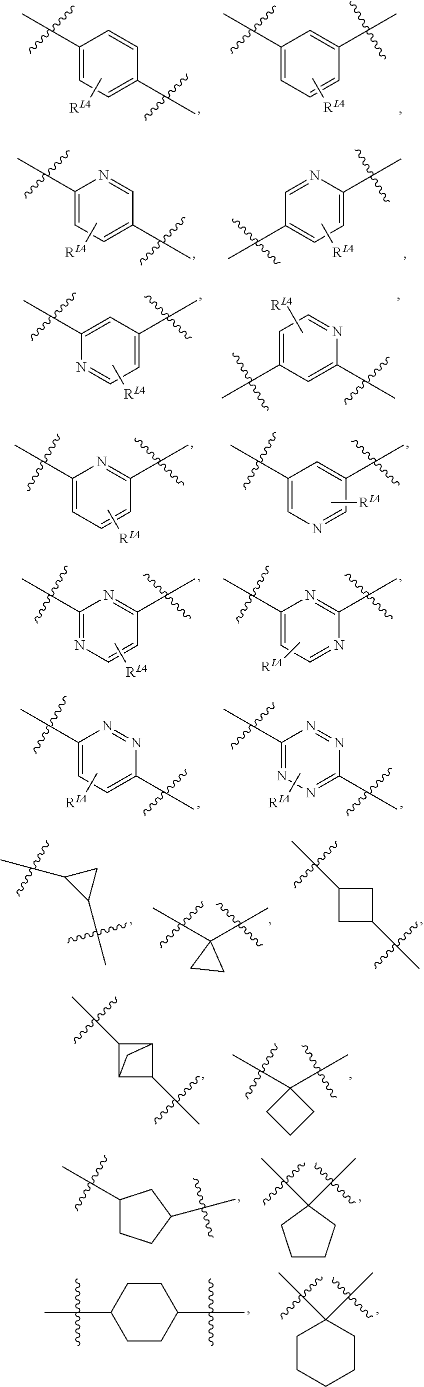

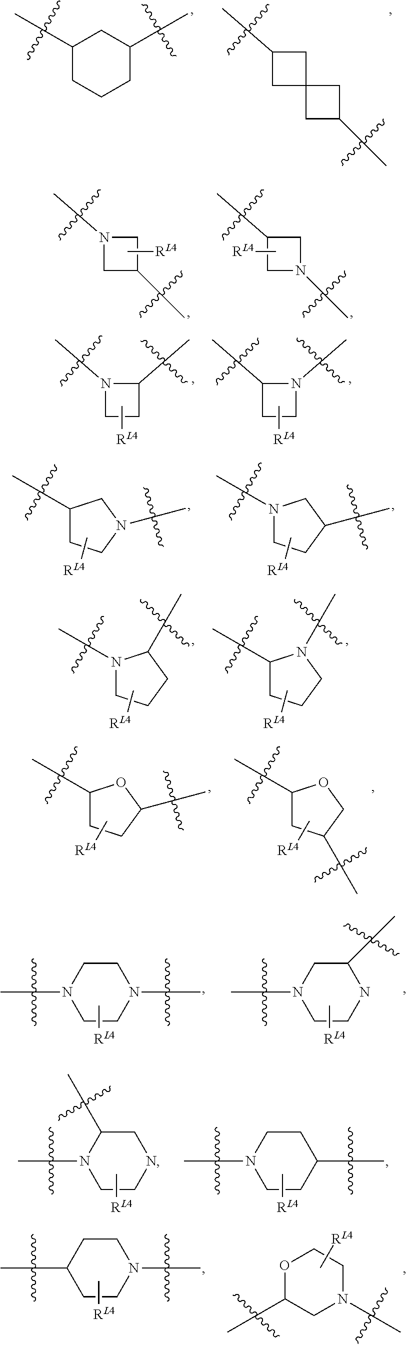

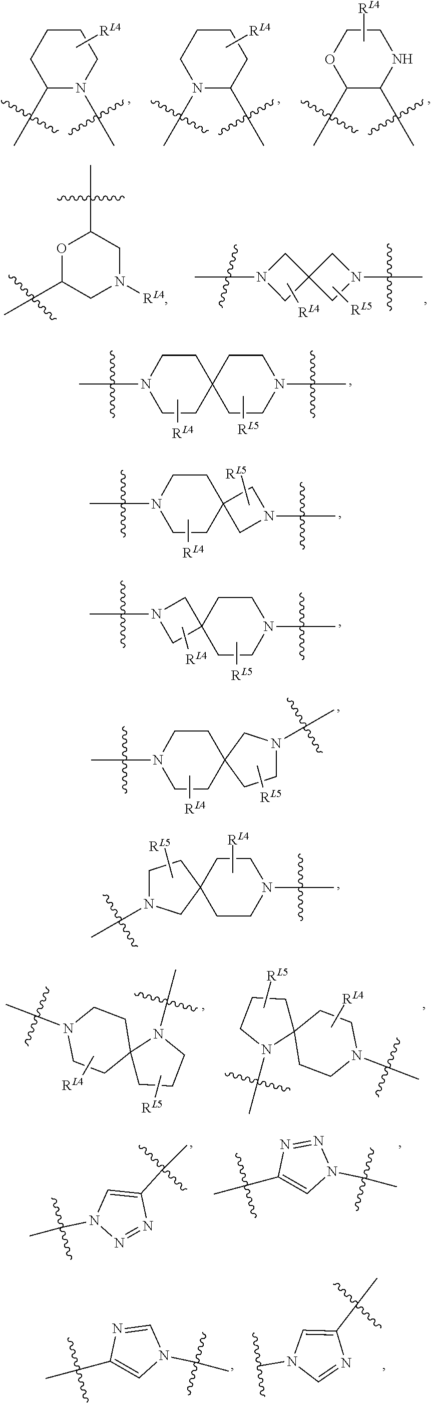

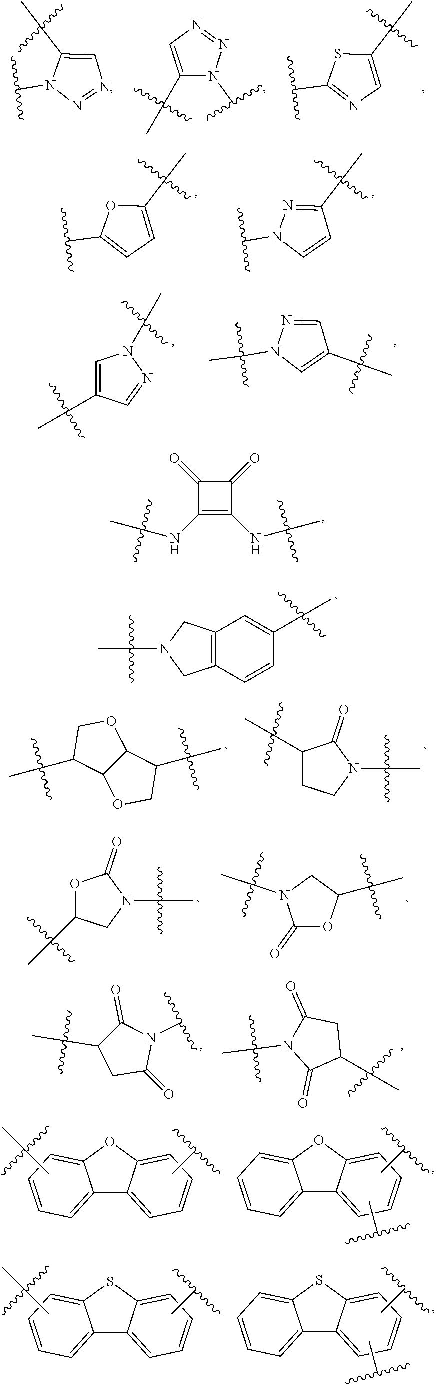

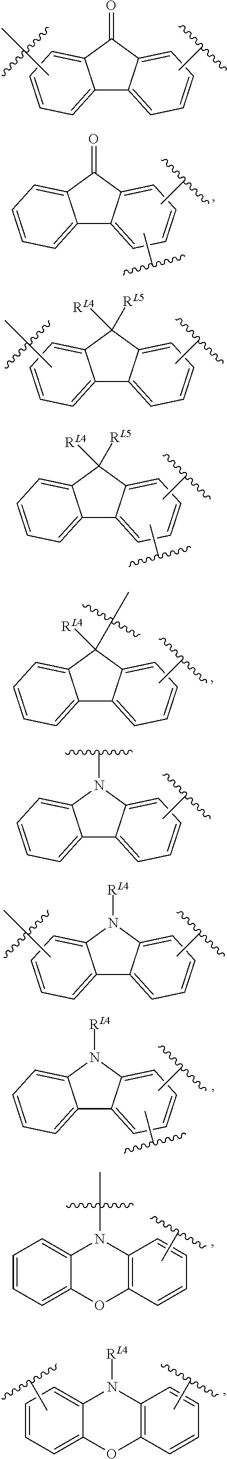

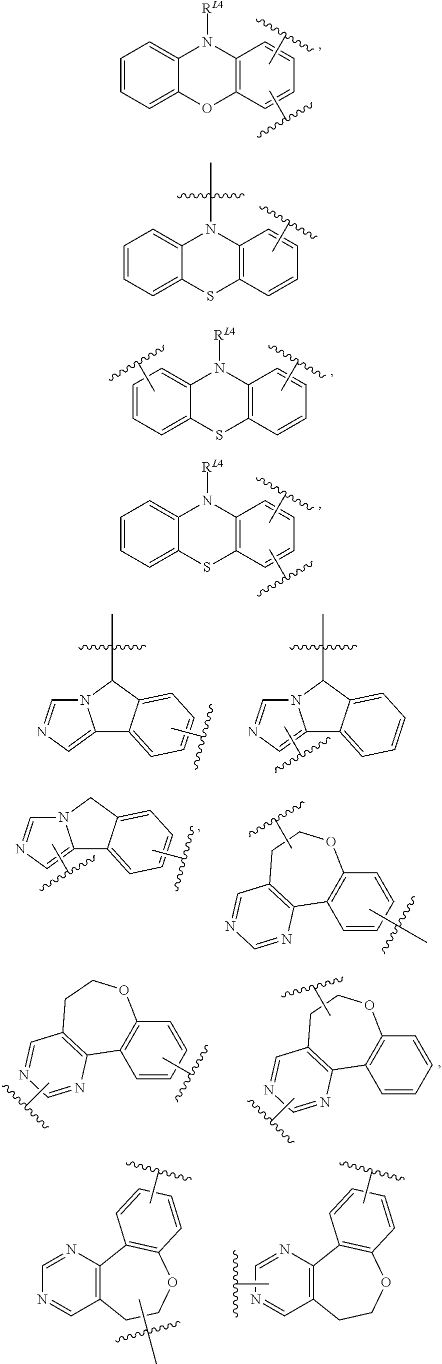

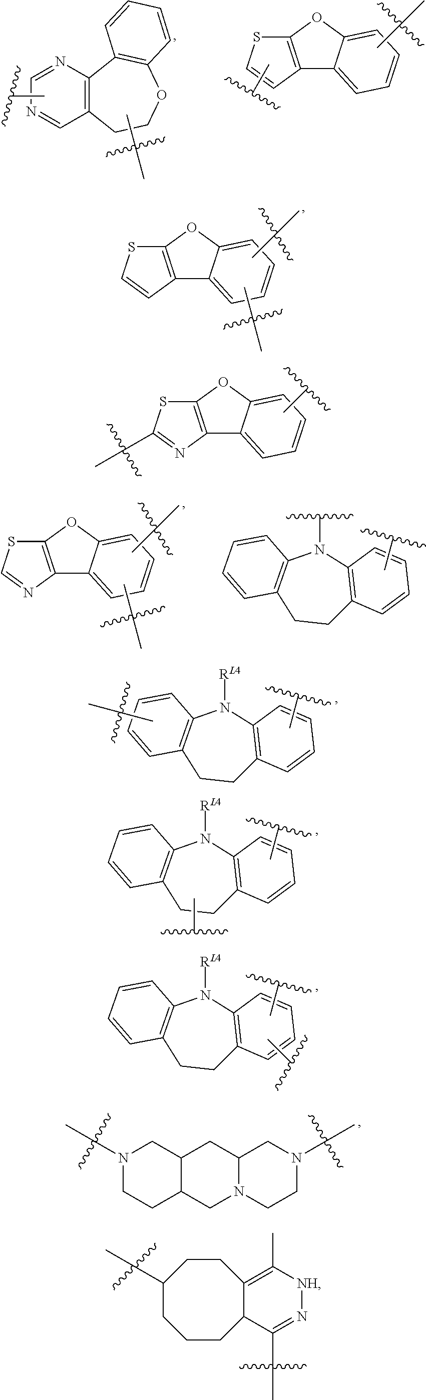

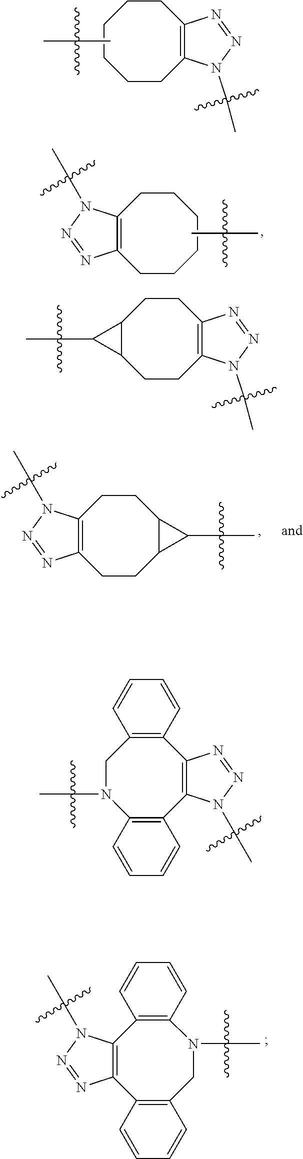

R^{L4} and R^{L5} are independently selected from: H, OH, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cyclohydrocarbyl, C₃₋₁₁ heterocyclohydrocarbyl, O(C₁₋₈ cyclohydrocarbyl), S(C₁₋₈ cyclohydrocarbyl), NH(C₁₋₈ cyclohydrocarbyl), N(C₁₋₈ cyclohydrocarbyl)(C₁₋₈ alkyl), OH, NH₂, SH, SO₂(C₁₋₈ alkyl), P(═O)(OC₁₋₈ alkyl)(C₁₋₈ alkyl), P(═O)(OC₁₋₈ alkyl)₂, C₁₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, Si(OH)₃, Si(C₁₋₈ alkyl)₃, Si(OH)(C₁₋₈ alkyl)₂, C(═O)(C₁₋₈ alkyl), CO₂H, CN, CF₃, CHF₂, CH₂F, NO₂, SF₅, SO₂NH(C₁₋₈ alkyl), SO₂N(C₁₋₈ alkyl)₂, S(═O)N(C₁₋₈ alkyl)₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl), NHSO₂N(C₁₋₈ alkyl)₂, and NHSO₂NH₂; or RM and R^{L5}, together with the atom to which they are attached, form cycloalkylene or heterocyclylene.

More specifically, R^{L4} and R^{L5} are independently selected from: —CH₃,

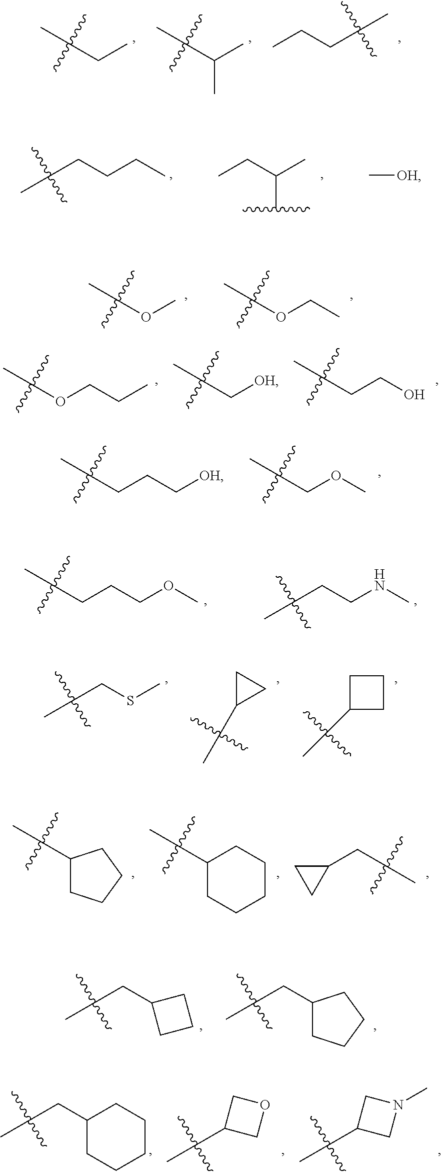

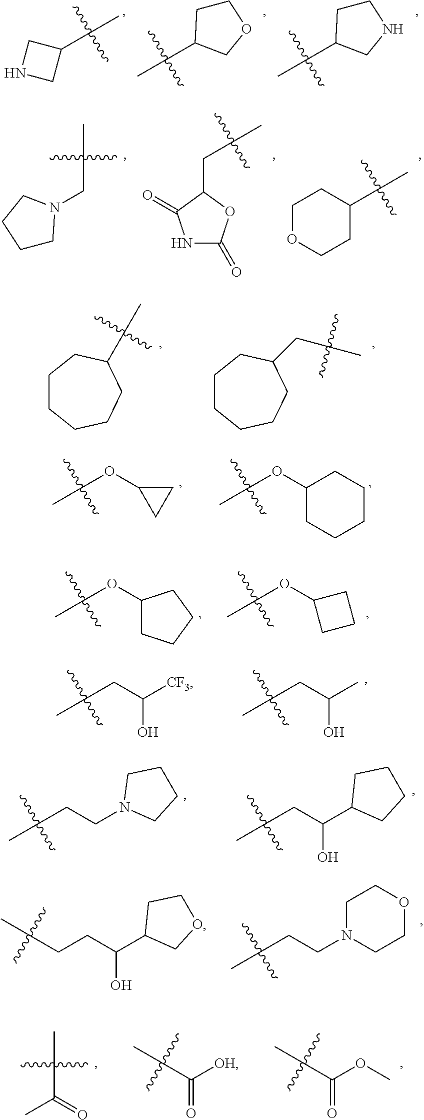

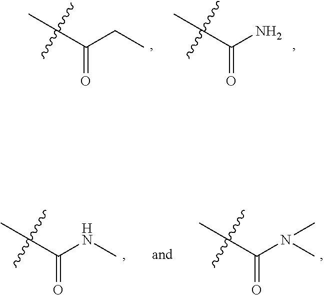

or R^{L4} and R^{L5}, together with the atom to which they are attached, form three- to six-membered cycloalkylene

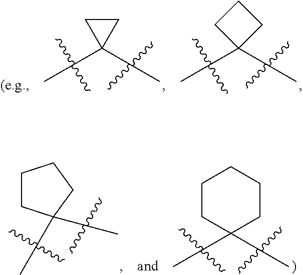 [SMILES: *C1(*)CC1 ; *C1(*)CCC1 ; *C1(*)CCCC1 ; *C1(*)CCCCC1]

or four- to six-membered heterocycloalkylene

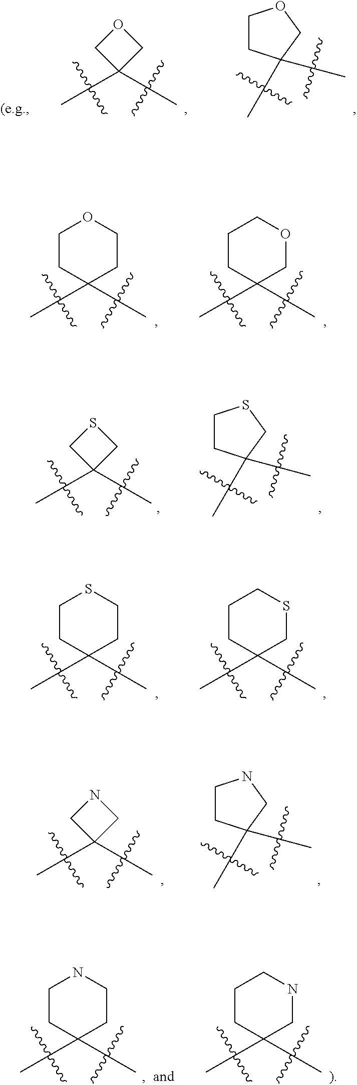

In some embodiments of the present disclosure, R^{L1} is H.

In some embodiments of the present disclosure, R^{L3} is H.

In some embodiments of the present disclosure, R^{L4} is H.

In some embodiments of the present disclosure, R^{L4} is OH.

In some embodiments of the present disclosure, R^{L5} is H.

In some embodiments of the present disclosure, R^{L5} is OH.

In one embodiment of the present disclosure, L₂ adopts the following scheme (1): L₂ is C1-C20 linear alkylene, wherein 0-6 methylene units in the alkylene are independently substituted with the following groups: —O—, —S—, —S—S—, —C(O)—, —C(O)O—, —OC(O)—, —N(R^{L2}), —C(O)N(R^{L2)}—, —N(R^{L2})C(O)—,

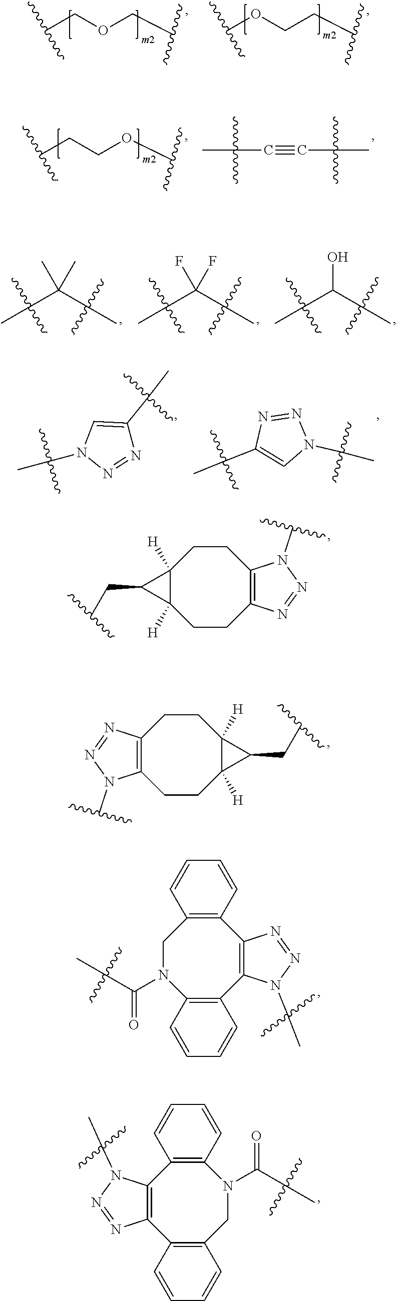

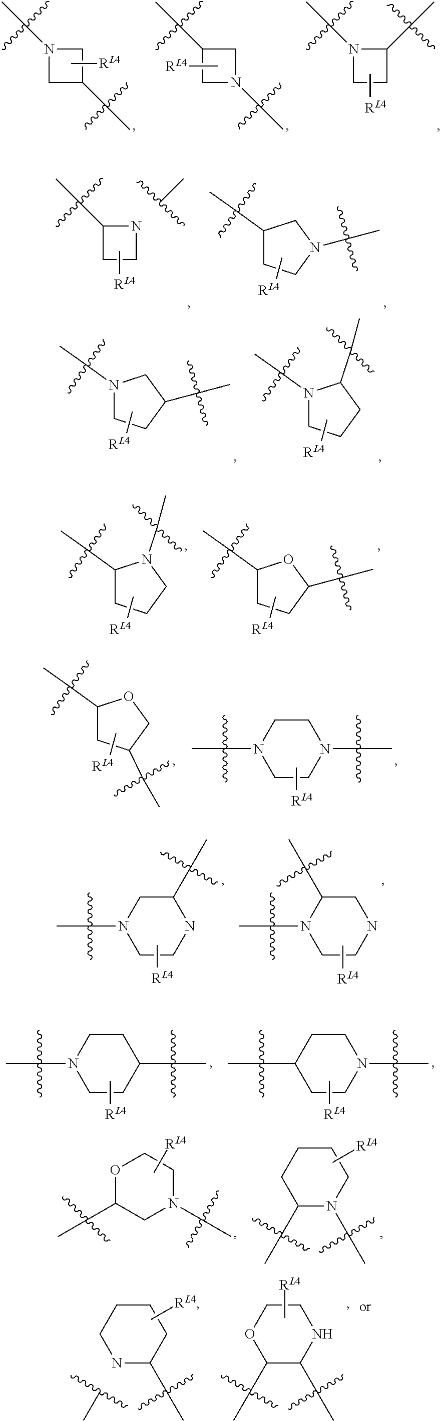

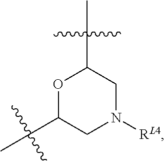

wherein, each R^{L2} is independently selected from: H and C₁-C₆ alkyl, and each R^{L} is independently selected from: OH and C₁-C₆ alkoxy.

More specifically, L₂ may be selected from: C₁-C₂₀ linear alkylene, —(CH₂CH₂O)ₘ₂—CH₂—, —(CH₂CH₂O)ₘ₂—CH₂CH₂—, —CH₂—(CH₂CH₂O)ₘ₂—CH₂—, —CH₂CH₂—(CH₂CH₂O)ₘ₂—CH₂—, —CH₂CH₂—(CH₂CH₂O)ₘ₂—CH₂CH₂—, —(C₁-C₁₀ alkylene)-O—(C₁-C₁₀ alkylene)-, —(C₁-C₁₀ alkylene)-NH—(C₁-C₁₀ alkylene)-, —(C₁-C₁₀ alkylene)-C(O)NH—(C₁-C₁₀ alkylene)-, —(C₁-C₁₀ alkylene)-NHC(O)—(C₁-C₁₀ alkylene)-, —(C₁-C₆ alkylene)-O—(C₁-C₆ alkylene)-C(O)NH—(C₁-C₆ alkylene)-, —(C₁-C₆ alkylene)-O—(C₁-C₆ alkylene)-NHC(O)—(C₁-C₆ alkylene)-,

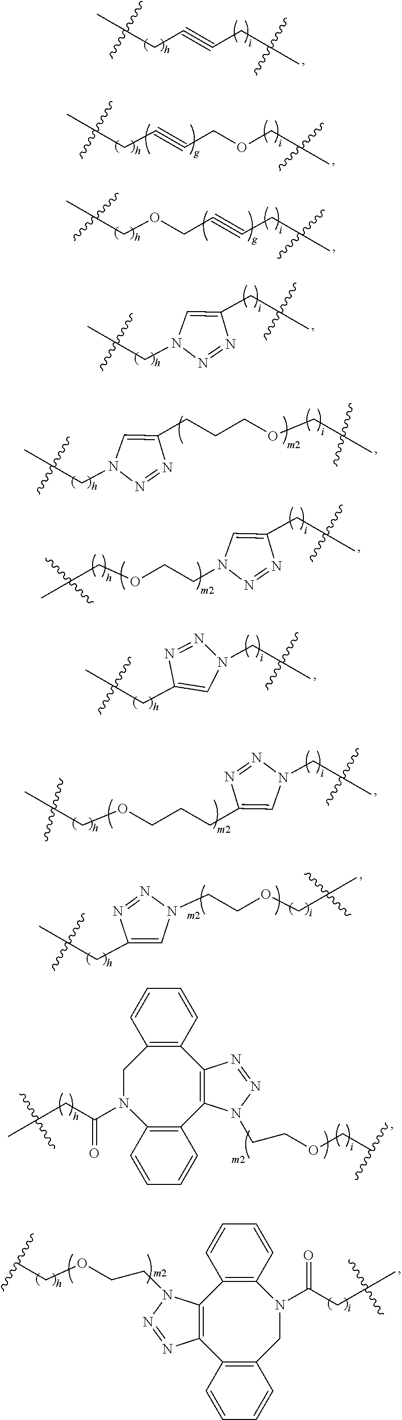

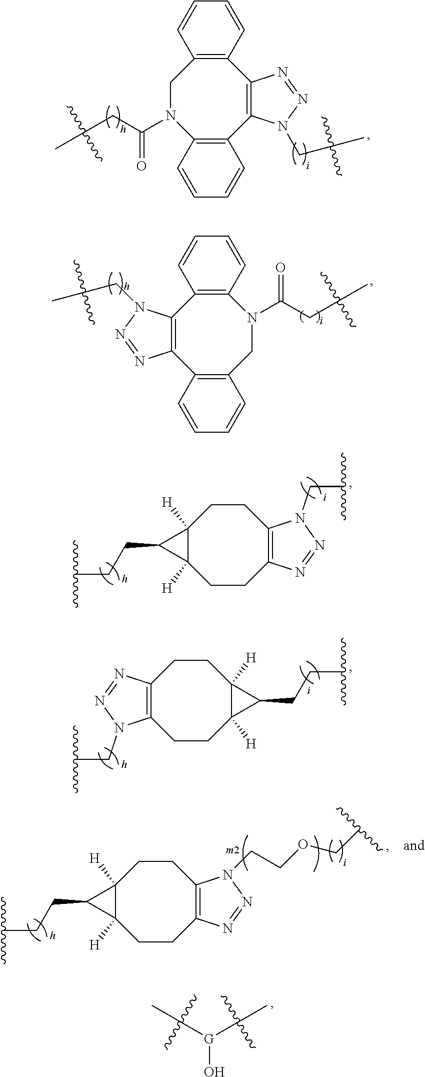

wherein m2 is selected from integers between 1 and 10, g is 0 or 1, h is selected from integers between 0 and 10 (e.g., 0, 1, 2, 3, 4, 5, 6, 7, 8, 9, or 10), i is selected from integers between 0 and 10 (e.g., 0, 1, 2, 3, 4, 5, 6, 7, 8, 9, or 10), and G is any suitable trivalent group.

In some specific embodiments of the present disclosure, L₂ is selected from:

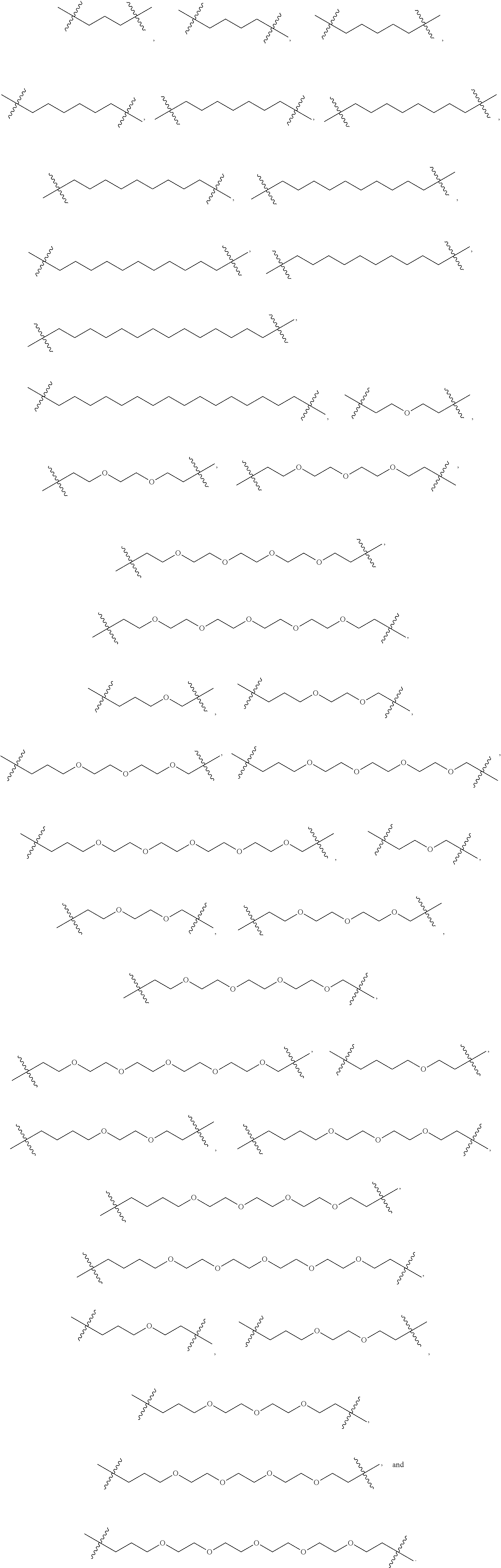

In some embodiments of the present disclosure, L₂ is selected from C₁-C₂₀ linear alkylene, —(C₀-C₆ alkylene)-(CH₂CH₂O)ₘ₂—(C₁-C₆ alkylene)-, —(C₁-C₁₀ alkylene)-O—(C₁-C₁₀ alkylene)-, —(C₁-C₁₀ alkylene)-NH—(C₁-C₁₀ alkylene)-, —(C₁-C₁₀ alkylene)-C(O)NH—(C₁-C₁₀ alkylene)-, —(C₁-C₁₀ alkylene)-NHC(O)—(C₁-C₁₀ alkylene)-, —(C₁-C₆ alkylene)-O—(C₁-C₆ alkylene)-C(O)NH—(C₁-C₆ alkylene)-, and —(C₁-C₆ alkylene)-O—(C₁-C₆ alkylene)-NHC(O)—(C₁-C₆ alkylene)-, wherein 1 methylene unit in the alkylene is independently substituted with a group capable of linking to a solid-phase support (e.g.,

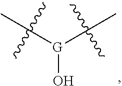

and G is any suitable trivalent group).

In some embodiments of the present disclosure,

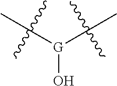

is

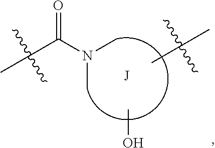

wherein ring J is a saturated 4- to 10-membered heterocyclic ring.

Specifically, ring J is a 4- to 6-membered saturated heterocyclic ring, and optionally, it further contains other heteroatoms, such as

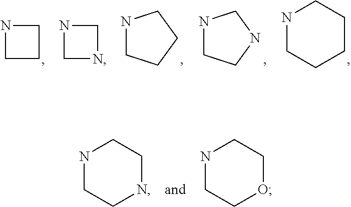

more specifically,

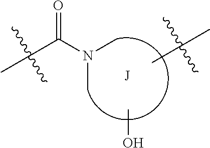

may be, for example,

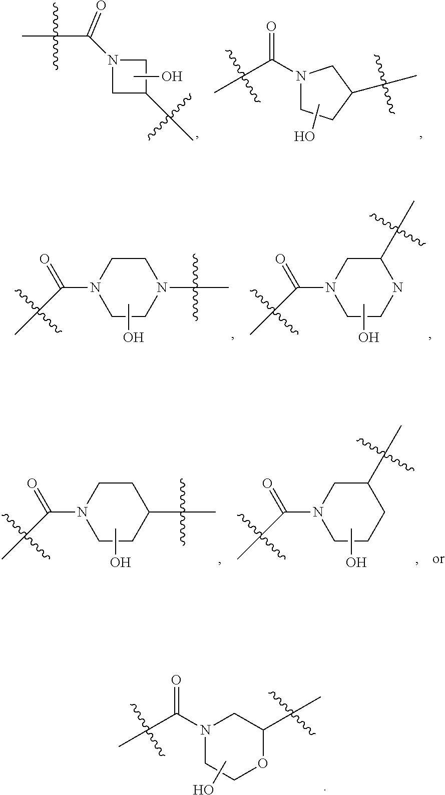

In some embodiments of the present disclosure, G is a sugar residue; for example,

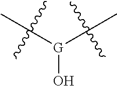

may be

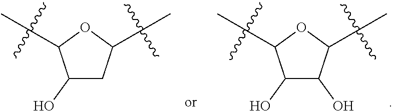 [SMILES: *C1CC(O)C(*)O1 ; *C1OC(*)C(O)C1O]

In some embodiments of the present disclosure, L₂ is

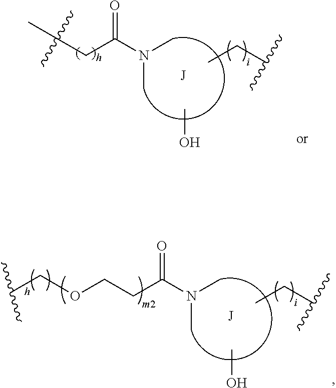

particularly

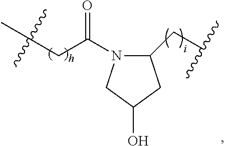

such as

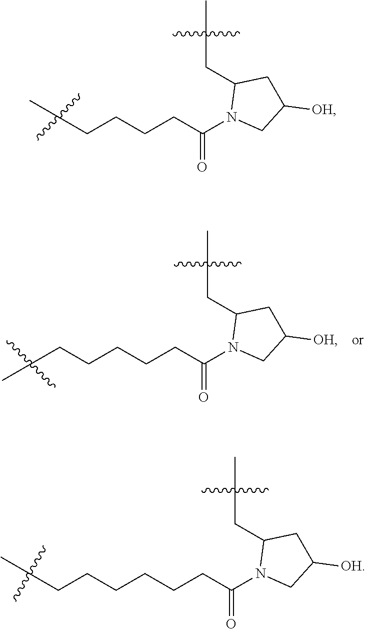 [SMILES: *CCCCC(=O)N1CC(O)CC1C* ; *CCCCCC(=O)N1CC(O)CC1C* ; *CCCCCCC(=O)N1CC(O)CC1C*]

In another embodiment of the present disclosure, L₂ adopts the following scheme (2): L₂ is C1-C20 linear alkylene, wherein 1-3 methylene units are independently substituted with the following groups: —CY—,

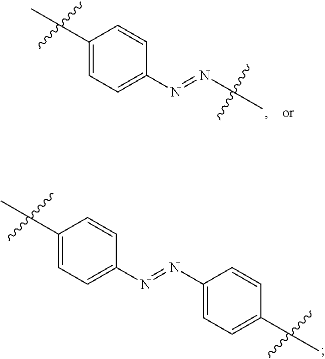 [SMILES: */N=N/c1ccc(*)cc1 ; *c1ccc(/N=N/c2ccc(*)cc2)cc1]

optionally, L₂ further comprises a group selected from: —O—, —C(O)—, —N(R^{L2})—, —C(O)N(R^{L2})—, —N(R^{L2})C(O)—,

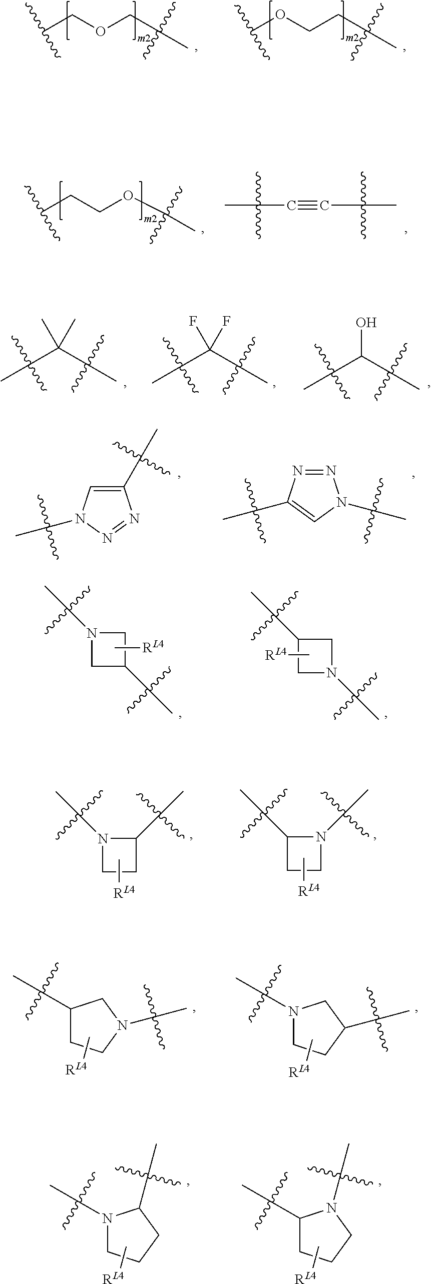

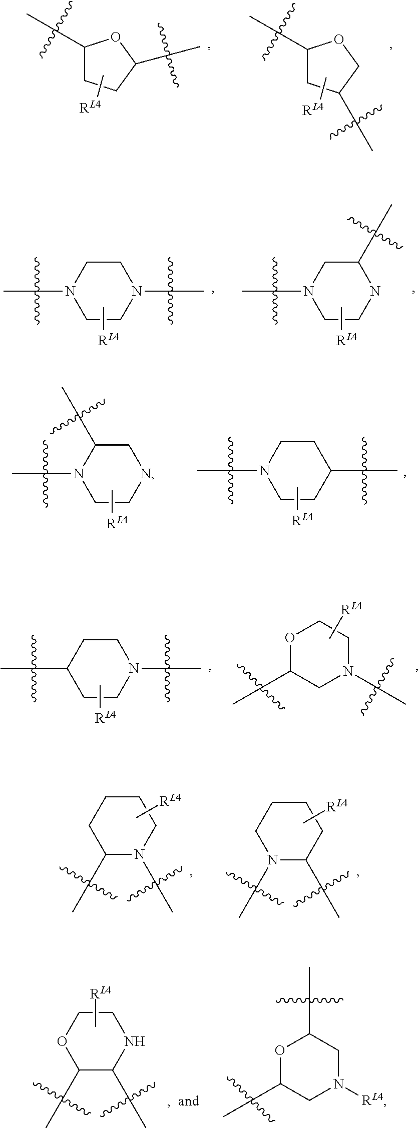

wherein, each R^{L2} is independently selected from: H and C₁-C₆ alkyl, and each R^{L4} is independently selected from: OH and C₁-C₆ alkoxy.

In some embodiments of the present disclosure, —CY— is selected from:

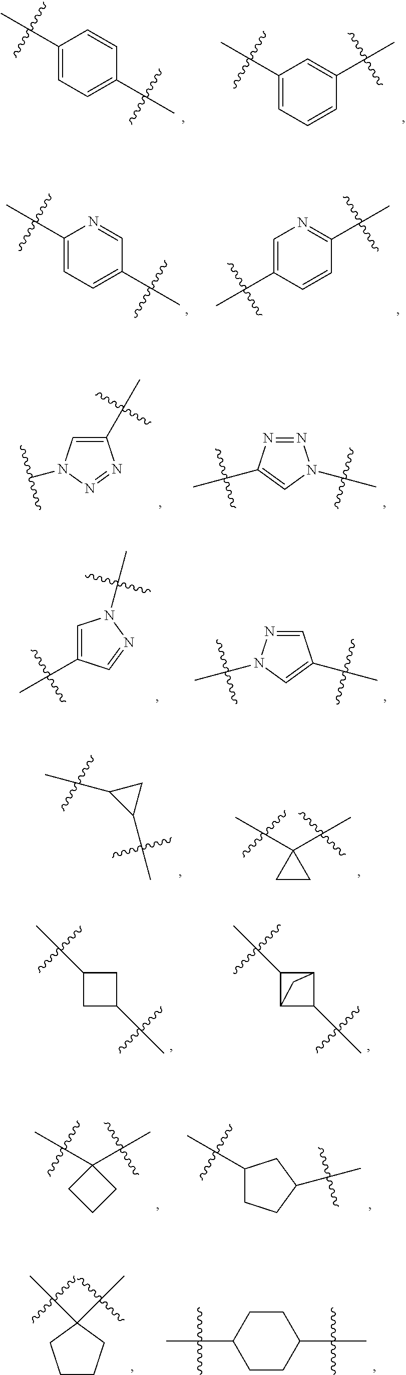

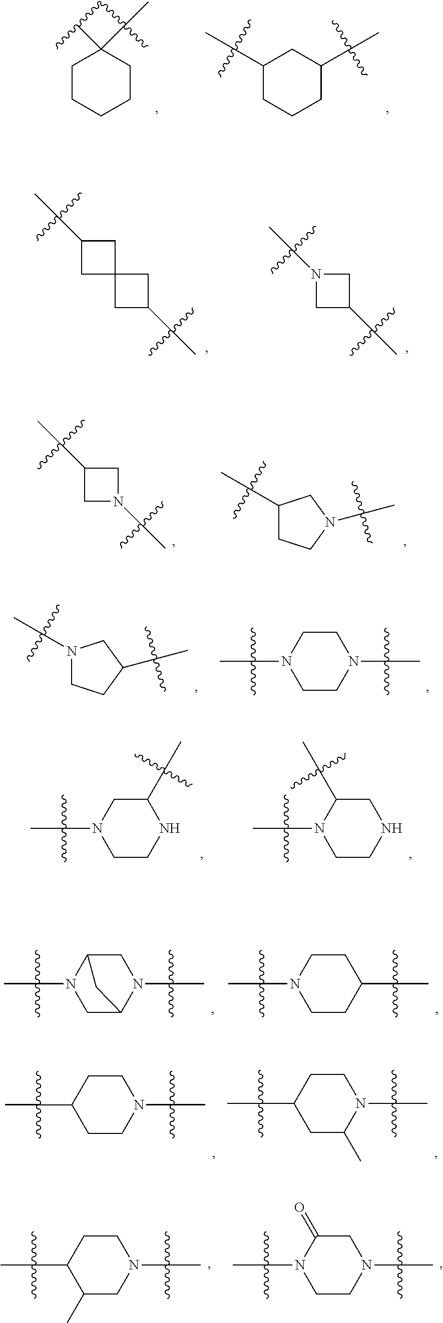

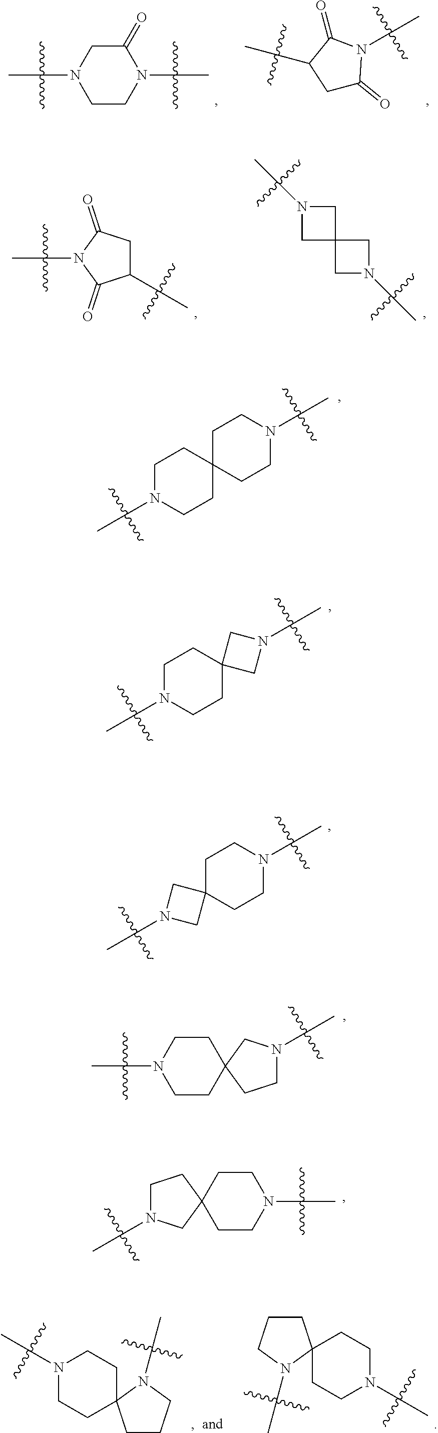

In some specific embodiments of the present disclosure, L₂ is selected from:

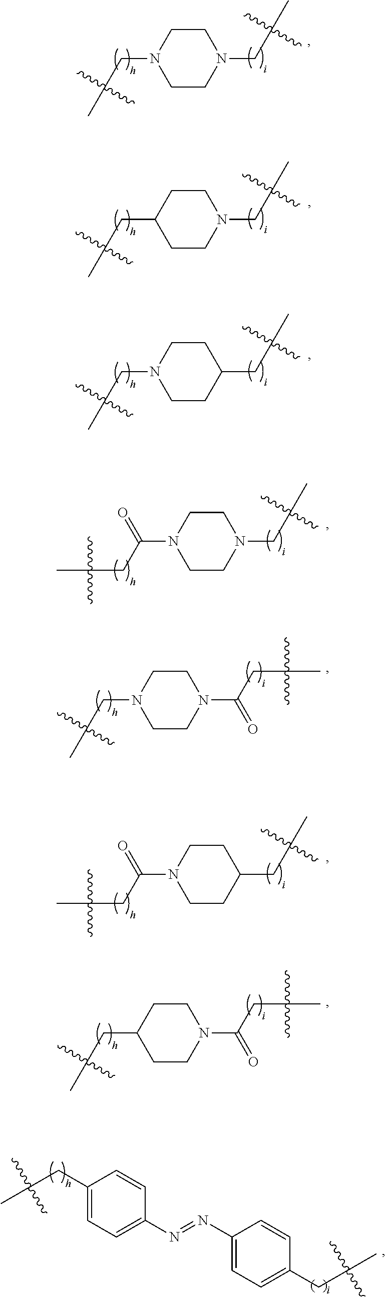

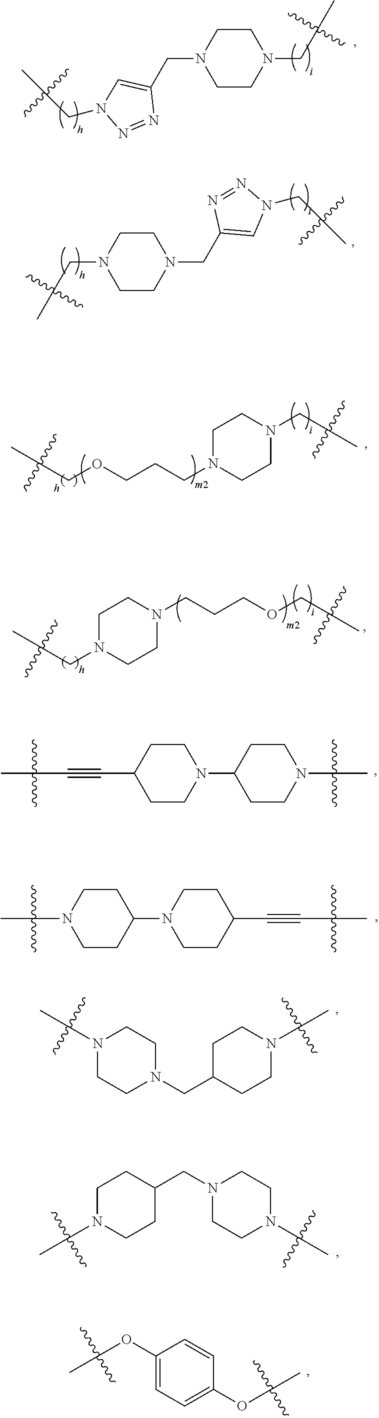

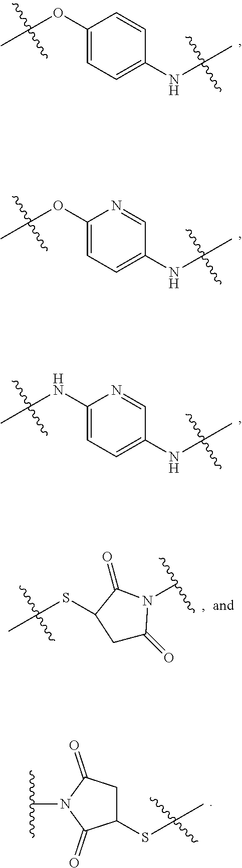 [SMILES: *Nc1ccc(O*)cc1 ; *Nc1ccc(O*)nc1 ; *Nc1ccc(N*)nc1 ; *SC1CC(=O)N(*)C1=O ; *SC1CC(=O)N(*)C1=O]

In another embodiment of the present disclosure, L₂ adopts the following scheme (3): L₂ is an oligonucleotide residue, such as a DNA oligonucleotide residue, an RNA oligonucleotide residue, or a DNA/RNA hybrid oligonucleotide residue, which may be single-stranded or double-stranded. In this case, L₁ may be a single bond or O, that is, the GRO

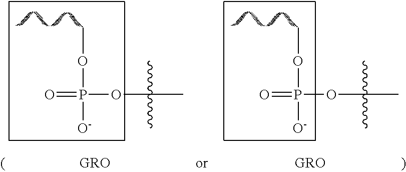

is linked to L₂ via a phosphoester bond

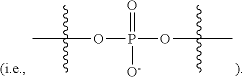 [SMILES: *OP(=O)([O-])O*]

Specifically, the oligonucleotide consists of A and/or T.

In some embodiments of the present disclosure, L₂ is a single-stranded oligonucleotide residue comprising 3-30 (e.g., 3, 4, 5, 6, 7, 8, 9, 10, 12, 14, 16, 18, 20, 22, 24, 26, 28, or 30) A or T, such as a single-stranded oligonucleotide residue consisting of 6 A.

In some embodiments of the present disclosure, L₂ is a double-stranded oligonucleotide residue comprising 3-30 (e.g., 3, 4, 5, 6, 7, 8, 9, 10, 12, 14, 16, 18, 20, 22, 24, 26, 28, or 30) A-T pairs, such as a double-stranded oligonucleotide residue consisting of 6 A-T pairs.

In some embodiments of the present disclosure, L₂ is a single-stranded DNA oligonucleotide residue consisting of 6 A.

In some embodiments of the present disclosure, L₂ is a double-stranded DNA oligonucleotide consisting of 6 A-T pairs.

In some embodiments of the present disclosure, L₂ is a double-stranded DNA oligonucleotide consisting of 10 A-T pairs.

In some embodiments of the present disclosure, L₂ is a double-stranded DNA/RNA hybrid oligonucleotide, wherein the DNA single strand consists of 6 T, and the RNA single strand consists of 6 A.

In another embodiment of the present disclosure, L₂ adopts the following scheme (4): L₂ is an oligopeptide residue consisting of 2-10 (e.g., 2, 3, 4, 5, 6, 7, 8, 9, or 10) amino acid residues.

Specifically, the target protein ligand may be a small molecule compound, an oligonucleotide, an oligopeptide, a polypeptide, a protein (e.g., an antibody), or the like.

In some embodiments of the present disclosure, the target protein ligand is an oligonucleotide, and L₂ adopts the scheme (3) described above, i.e., an oligonucleotide residue; in addition, L₁ and L₃ may be a single bond or O, that is, the GRO and the target protein ligand are each linked to L₂ via a phosphoester bond

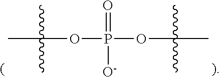 [SMILES: *OP(=O)([O-])O*]

In some embodiments of the present disclosure, the target protein ligand is a small molecule compound, and L₂ adopts the scheme (1) and/or scheme (2) described above.

In some embodiments of the present disclosure, the target protein ligand is an oligonucleotide, such as a DNA oligonucleotide, an RNA oligonucleotide, or a DNA/RNA hybrid oligonucleotide, which may be single-stranded or double-stranded.

In some embodiments of the present disclosure, the target protein ligand is a single-stranded oligonucleotide, and L may be linked to any base, sugar, or phosphate backbone at the 3′ end or 5′ end or in the middle of the oligonucleotide strand, particularly at the 3′ end or 5′ end.

In some embodiments of the present disclosure, the target protein ligand is a double-stranded oligonucleotide, and L may be linked to any base, sugar, or phosphate backbone at the 3′ end or 5′ end or in the middle of the sense strand of the oligonucleotide, or any base, sugar, or phosphate backbone at the 3′ end or 5′ end or in the middle of the antisense strand of the oligonucleotide, particularly at the 3′ end or 5′ end.

Specifically, the target protein is a protein that needs to be targeted for degradation, including but not limited to, mutant proteins, pseudokinase, transcription factors, scaffold proteins, membrane proteins, and the like, such as the target proteins shown as follows:

| | | | | |
|---|---|---|---|---|
| FAK | USP7 | GPX4 | PI3K | JAK |
| MetAP-2 | ER | AR | BET | IKAROS |
| RTK | ALC | EGFR | HER2 | TRK |
| BTK | STAT | IRAK4 | BCL-XL | TBK1 |
| FRS2 | STAT3 | HDAC6 | PDEδ | MAFF |
| Tau | GSK-3 | α-syn | mHtt | TDP-43 |
| FUS | SOD | NS3/4A | RIPK2 | BCR-ABL |
| PCAF/GCN5 | SHP2 | Bcl-2 | PD-L1 | NAMPT |
| EGFR-L858R | NA | STING | FKBP12 | TK |
| BRDT | FAP | Hsp70 | NLRP3 | cGAS |
| FLT3-ITD | c-MET | ALK | Akt | CK2 |
| FLT3 | Sirt2 | Pirin | SMAD3 | ARNT |
| CDK2 | CDK4 | CDK6 | CDK9 | ERK1 |
| BRD2 | BRD4 | BRD6 | BRD9 | ERK2 |
| IKZF1 | IKZF3 | RAR | Bcr/Abl | c-Abl |
| DAPK1 | PSD-95 | TRIM24 | CRABP-I | CRABP-II |
| TACC3 | AHR | FKBP12 | ERRα | X protein |
| Huntingtin | DHODH | Halo Tags | NQO1 | GSTP1 |
| CDK12 | PARP1 | p53 | HMGCR | LXRs |
| VEGFR-2 | SMARCA4 | EED | SMARCA2 | SGK3 |
| EGFR/PARP | ERK5 | KRAS | EZH2 | MEK |
| ER/GPER | XPO1 | Myc | VEGF | |

In some embodiments of the present disclosure, the target protein is a transcription factor protein, such as SIX-1, HIF-1, RUNX2, FOXO3, RUNX1, AR, ER, STAT3, Myc, LZTFL1, RFX1, RFX1, MITF, KLF4, p53, RUNX3, FOXO1/3/4, TFAM, NRF2, XBP1, NF-κB, BRD4, and VEGF.

In one embodiment of the present disclosure, the target protein ligand is a c-MET ligand, such as DNA aptamer SL1 having the nucleotide sequence set forth in SEQ ID NO: 19: 5′-ATCAGGCTGGATGGTAGCTCGGTCGGGGTGGGTGGGTTGGCAAGTCTGAT-3′.

In some embodiments of the present disclosure, in the targeted protein degrader, the PI moiety is DNA aptamer SL1 having the nucleotide sequence set forth in SEQ ID NO: 19, wherein L is a double-stranded DNA oligonucleotide residue (e.g., one strand consists of 6 T, and the other strand consists of 6 A), and the 5′ end or 3′ end of the PI moiety is linked to the 5′ end or 3′ end of the GRO via L.

In one embodiment of the present disclosure, the target protein ligand is a STAT3 protein ligand, such as double-stranded transcription factor decoy oligonucleotide S3 that specifically recognizes STAT3, wherein the sense strand S3-F of S3 is set forth in SEQ ID NO: 5: 5′-CTTCTGGGAAA-3′, and the antisense strand S3-R of S3 is set forth in SEQ ID NO: 6: 5′-TTTCCCAGAAG-3′.

In some embodiments of the present disclosure, in the targeted protein degrader, the PI moiety is double-stranded transcription factor decoy oligonucleotide S3 that specifically recognizes STAT3, wherein the sense strand S3-F of S3 is set forth in SEQ ID NO: 5, and the antisense strand S3-R of S3 is set forth in SEQ ID NO: 6, wherein L is a single-stranded oligonucleotide residue (e.g., a single-stranded DNA oligonucleotide linker consisting of 6 A), and the sense strand or antisense strand of the PI moiety is linked to the 5′ end or 3′ end of the GRO via L.

In one embodiment of the present disclosure, the target protein ligand is a ligand of Myc (e.g., c-Myc), such as a double-stranded transcription factor decoy oligonucleotide, wherein the sense strand sequence MYC-F of the oligonucleotide is set forth in SEQ ID NO: 7: 5′-GAGCACGTGGT-3′, and the antisense strand sequence MYC-R of the oligonucleotide is set forth in SEQ ID NO: 8: 5′-ACCACGTGCTC-3′.

In some embodiments of the present disclosure, in the targeted protein degrader, the PI moiety is a double-stranded transcription factor decoy oligonucleotide that specifically recognizes c-Myc, wherein the sense strand sequence MYC-F of the oligonucleotide is set forth in SEQ ID NO: 7, and the antisense strand sequence MYC-R of the oligonucleotide is set forth in SEQ ID NO: 8, wherein L is a single-stranded oligonucleotide residue (e.g., a single-stranded DNA oligonucleotide linker consisting of 6 A), and the sense strand or antisense strand of the PI moiety is linked to the 5′ end or 3′ end of the GRO via L.

In one embodiment of the present disclosure, the target protein ligand is a ligand of a p53 mutant (e.g., p53-R175H, G245S, R248W/Q, R249S, R273C/H, R282W, etc.), such as RNA aptamer R175Hapt having the sequence set forth in SEQ ID NO: 9: 5′-AUUAGCGCAUUUUAACAUAGGGUGC-3′.

In some embodiments of the present disclosure, in the targeted protein degrader, the PI moiety is the RNA aptamer R175Hapt of p53-R175H, and R175Hapt has the sequence set forth in SEQ ID NO: 9, wherein L is a double-stranded oligonucleotide residue (e.g., one strand consists of 6 T, and the other strand consists of 6 A), and the 5′ end or 3′ end of the PI moiety and the 5′ end or 3′ end of the GRO are linked to the two strands of L, respectively.

In one embodiment of the present disclosure, the target protein ligand is a ligand of a splice mutant of AR (e.g., AR-V7), such as RNA oligonucleotide IncRV set forth in SEQ ID NO: 10: 5′-CCUUUUGUUUUUCCCUCUCCAGG-3′ or lncV7 set forth in SEQ ID NO: 11: 5′-UAUUUUUCCCUCUCCACCCU-3′.

In some embodiments of the present disclosure, in the targeted protein degrader, the PI moiety is RNA oligonucleotide lncRV or lncV7 having the sequence set forth in SEQ ID NO: 10 or 11, wherein L is a double-stranded oligonucleotide residue (e.g., one strand consists of 6 T, and the other strand consists of 6 A), and the 5′ end or 3′ end of the PI moiety and the 5′ end or 3′ end of the GRO are linked to the two strands of L, respectively.

In one embodiment of the present disclosure, the target protein ligand is a ligand of VEGF (such as VEGF-A (e.g., VEGF121, VEGF145, VEGF148, VEGF183, VEGF165, VEGF189, VEGF206), VEGF-B, VEGF-C, VEGF-D, or VEGF-E), such as single-stranded DNA aptamer V7t1 having the sequence set forth in SEQ ID NO: 12: 5′-TGTGGGGGTGGACGGGCCGGGTAGA-3′.

In some embodiments of the present disclosure, in the targeted protein degrader, the PI moiety is the single-stranded DNA aptamer V7t1 of VEGF165, and V7t1 has the sequence set forth in SEQ ID NO: 12, wherein L is a single-stranded oligonucleotide residue (e.g., a single-stranded oligonucleotide linker consisting of 6 A), and the PI moiety is linked to the 5′ end or 3′ end of the GRO via L.

In one embodiment of the present disclosure, the target protein ligand is a ligand of MDM2, such as the GRO (e.g., AS1411) described in the first objective of the present disclosure, or other MDM2 ligands known in the prior art.

In some embodiments of the present disclosure, in the targeted protein degrader, the PI moiety is selected from sequences set forth in SEQ ID NOs: 1-4 and 20-47, wherein L is a double-stranded oligonucleotide residue (e.g., one strand consists of 6 T, and the other strand consists of 6 A), and the 5′ end or 3′ end of the PI moiety and the 5′ end or 3′ end of the GRO are linked to the two strands of L, respectively.

In some embodiments of the present disclosure, the target protein ligand is a c-MET ligand, such as the compound of formula I described in patent documents WO2010072296A1 and CN101743241A (which are incorporated herein by reference in their entireties), particularly compounds A1-A314 in claim 14 of CN101743241A.

In one embodiment of the present disclosure, the c-MET ligand is tepotinib.

In some embodiments of the present disclosure, the targeted protein degrader represented by general formula I has the following structure:

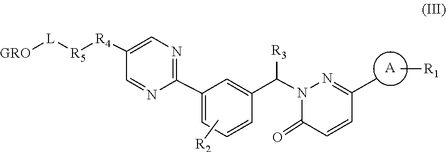

wherein
ring A is an aromatic ring or a heterocyclic ring;
R₁ is one or more independent substituents on ring A and is selected from: halogen, —CN, —NO₂, —OCF₃, C₀₋₁₀ alkyl, C₁₋₁₀ haloalkyl, —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —OC₀₋₁₀ alkyl, —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, and 4- to 10-membered heterocyclyl;
R₂ is one or more independent substituents on a benzene ring and is selected from: halogen, —CN, —NO₂, —CF₃, —OCF₃, C₀₋₁₀ alkyl, C₁₋₁₀ haloalkyl, —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —OC₀₋₁₀ alkyl, —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, and 4- to 10-membered heterocyclyl;
R₃ is selected from: C₀₋₁₀ alkyl and C₁₋₁₀ haloalkyl;
R₄ is —(C₀₋₆ alkyl)-R₄′—(C₀₋₆ alkyl)-, wherein R₄′ is selected from: a single bond, O, S, N(C₀₋₁₀ alkyl), C(O), OC(O), C(O)O, N(C₀₋₁₀ alkyl)C(O), C(O)N(C₀₋₁₀ alkyl), SO₂, SO₂N(C₀₋₁₀ alkyl),

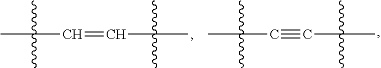

C₃₋₆ cycloalkylene, and 4- to 10-membered heterocyclylene;
R₅ is selected from: a single bond, O, S, N(C₀₋₁₀ alkyl), C(O), OC(O), C(O)O, N(C₀₋₁₀ alkyl)C(O), C(O)N(C₀₋₁₀ alkyl), SO₂, SO₂N(C₀₋₁₀ alkyl), C₃₋₆ cycloalkylene, and 4- to 10-membered heterocyclylene (particularly 4- to 6-membered heterocycloalkylene).

Specifically, ring A is a benzene ring, a naphthalene ring, biphenyl, piperidine, piperazine, pyrrolidine, morpholine, furan, thiophene, pyrrole, imidazole, pyrazole, oxazole, isoxazole, thiazole, isothiazole, pyridine, pyrimidine, triazole, tetrazole, oxadiazole, thiadiazole, pyridazine, pyrazine, benzimidazole, benzotriazole, indole, benzo-1,3-dioxole, indazole, azabicyclo[3.2.1]octane, azabicyclo[2.2.2]octane, imidazolidine, azepane, or benzo-2,1,3-thiadiazole.

In some embodiments of the present disclosure, moiety

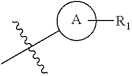

has the following structures:

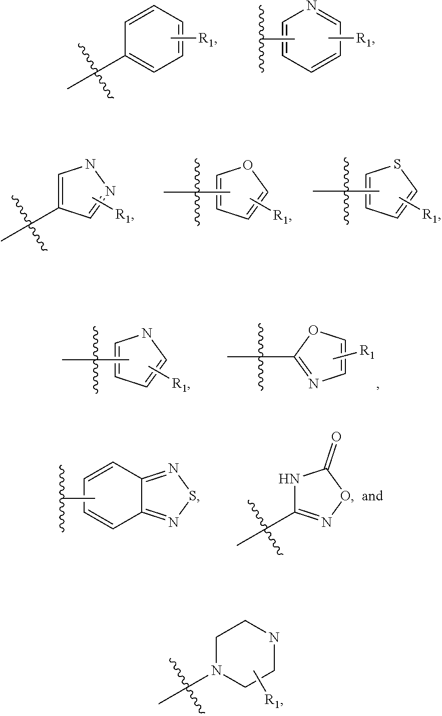

particularly

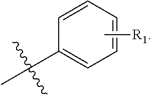

Specifically, R₁ is selected from: H, halogen (e.g., F and Cl), —CN, —NO₂, —OCF₃, C₁₋₃ alkyl, C₁₋₃ haloalkyl, —OC₀₋₃ alkyl, and —N(H)CO(C₁₋₃ alkyl); in some embodiments of the present disclosure, R₁ is —CN; in some embodiments of the present disclosure, R₁ is F.

Specifically, R₂ is selected from: H and halogen (e.g., F and Cl); in some embodiments of the present disclosure, R₂ is H.

Specifically, R₃ is selected from: H and C₁₋₃ alkyl; in some embodiments of the present disclosure, R₃ is H.

Specifically, R₄′ is selected from: a single bond, O, S, N(H), C(O), N(H)C(O), C(O)N(H),

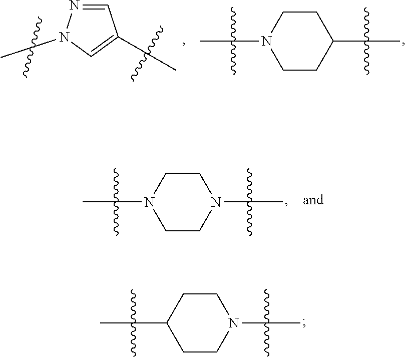 [SMILES: *c1cnn(*)c1 ; *C1CCN(*)CC1 ; *N1CCN(*)CC1 ; *C1CCN(*)CC1]

in some embodiments of the present disclosure, R₄ is O.

In some embodiments of the present disclosure, R₄ is —(C₁₋₆ alkyl)-O—.

Specifically, R₅ is selected from: a single bond, O, S, N(C₀₋₃ alkyl), N(H)C(O), C(O)N(H),

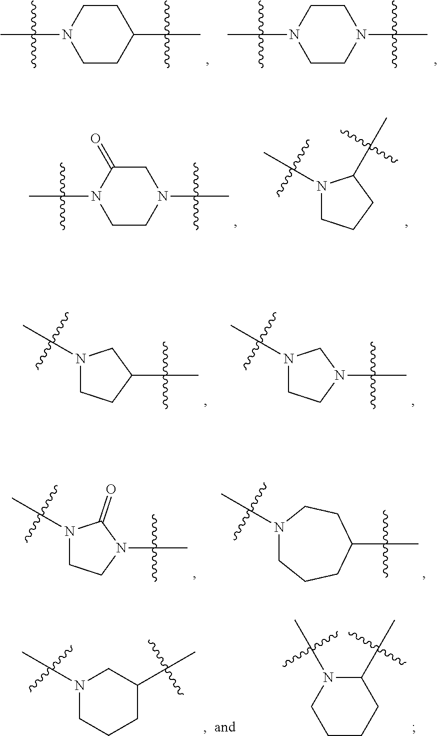

in some embodiments of the present disclosure, R₅ is

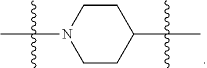 [SMILES: *C1CCN(*)CC1]

In some embodiments of the present disclosure, the targeted protein degrader represented by general formula I has the following structure:

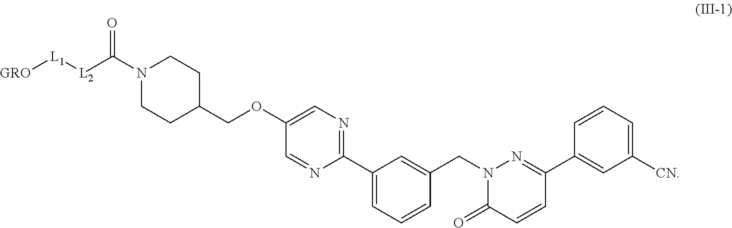

Specifically, in general formula III-1, L₂ adopts the scheme (1) and/or scheme (2) described above, particularly scheme (1), for example,

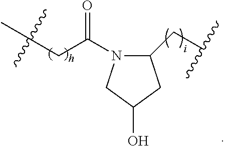

Specifically, in general formula III-1, L₁ may be a single bond or O.

In one embodiment of the present disclosure, the targeted protein degrader has the following structure:

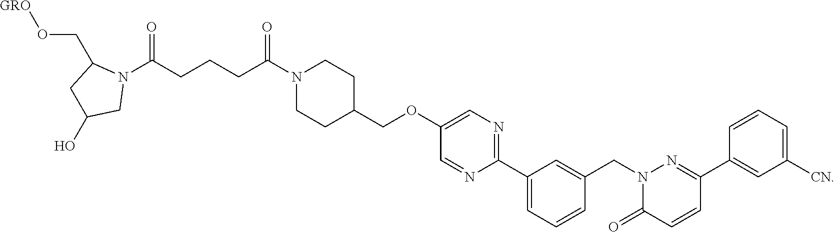

In some embodiments of the present disclosure, the c-MET ligand may also be, for example, crizotinib, capmatinib, savolitinib, AMG-337, cabozantinib (XL-184), foretinib, merestinib, BMS-777607, golvatinib, sitravatinib, glesatinib, BMS-794833, tivantinib (ARQ-197), or the like.

Specifically, the PI moiety in general formula I may also have the following structures:

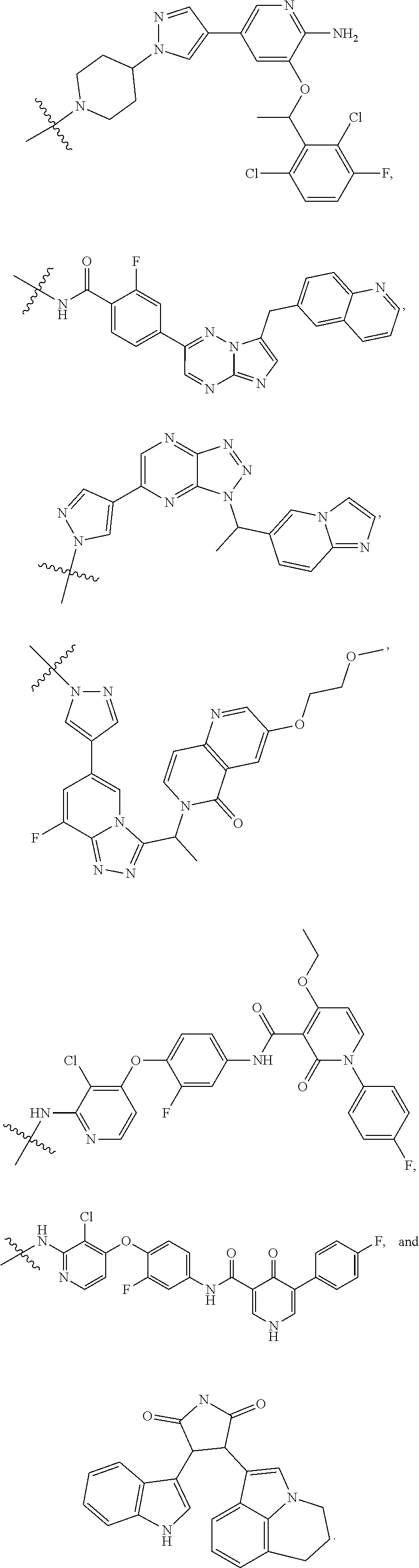

In another embodiment of the present disclosure, the targeted protein degrader has the following structure:

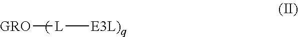

wherein
L is a linker;
E3L is an E3 ubiquitin ligase ligand moiety;
q is an integer of 1-100 (e.g., 1, 2, 3, 4, 5, 6, 7, 8, 9, 10, 12, 14, 15, 20, 25, 30, 35, 40, 50, 60, 80, or 100).

In a preferred embodiment of the present disclosure, q is 1, that is, general formula II is GRO-L-E3L.

Specifically, L₂ is described as above, particularly a chemical linker, such as the scheme (1) or (2) described above, particularly scheme (1).

Specifically, the E3 ligase ligand may be a small molecule compound, an oligonucleotide (including a nucleic acid aptamer, etc.), an oligopeptide, a polypeptide, a protein (e.g., an antibody), or the like.

Specifically, the E3 ligase ligand is selected from: a CRBN protein ligand, a VHL protein ligand, an MDM2 protein ligand, an IAP protein ligand, a cbl-b protein ligand, a DCAF15 protein ligand, a DCAF16 protein ligand, a KEAP1 protein ligand, an RNF4 protein ligand, an RNF114 protein ligand, a β-TrCP protein ligand, and an FEM1B protein ligand.

Specifically, the CRBN protein ligand includes: an amide compound, a phthalimide compound, thalidomide or a derivative thereof, lenalidomide or a derivative thereof, and pomalidomide or a derivative thereof.

Specifically, the VHL protein ligand may be VH032 or a derivative thereof.

Specifically, the MDM2 protein ligand may be Nutlin-3a or a derivative thereof, or the GRO described above.

Specifically, the IAP protein ligand may be Bestatin, LCD-161, MV-1, or a derivative thereof.

In some embodiments of the present disclosure, the E3L moiety in general formula II has the following structures:

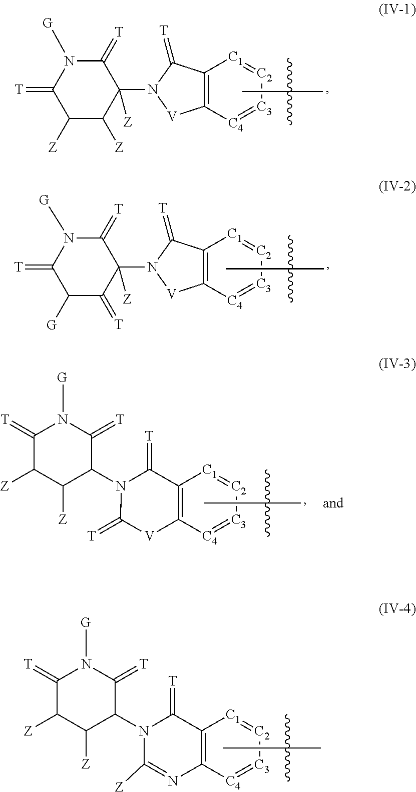

wherein C₁, C₂, C₃, and C₄ are independently selected from: CR¹⁰¹ and N, wherein each R¹⁰¹ is selected from: —OH, halogen, —CN, —NO₂, —CF₃, —OCF₃, C₀₋₁₀ alkyl, —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —OC₀₋₁₀ alkyl, —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, —O heterocycloalkyl, —N heterocycloalkyl, —S heterocycloalkyl, —N heterocycloaryl, —O heterocycloaryl, and —S heterocycloaryl;
T is selected from: O and S;
V is selected from: O, S,

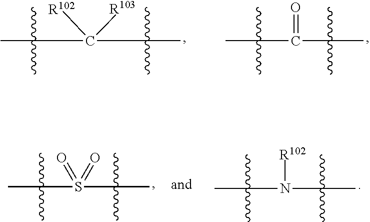

wherein R¹⁰² and R¹⁰³ are independently selected from: C₀₋₁₀ alkyl, cycloalkyl, and heterocycloalkyl;
G and Z are independently selected from: —OH, C₀₋₁₀ alkyl, C₃₋₁₀ cycloalkyl, 0-containing heterocycloalkyl, N-containing heterocycloalkyl, and S-containing heterocycloalkyl.

In one embodiment of the present disclosure, C₁, C₂, C₃, and C₄ are all C.

In another embodiment of the present disclosure, C₁ is C, and at least one of C₂, C₃, and C₄ is N.

In another embodiment of the present disclosure, C₂ is C, and at least one of C₁, C₃, and C₄ is N.

In another embodiment of the present disclosure, C₃ is C, and at least one of C₁, C₂, and C₄ is N.

In another embodiment of the present disclosure, C₄ is C, and at least one of C₁, C₂, and C₃ is N.

In one embodiment of the present disclosure, T is O.

Specifically, V is selected from: —CH₂—,

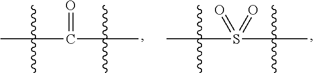

### and —NH—.

Specifically, G and Z are independently selected from: —H, —CH₃ and —CH₂CH₃.

Specifically, the E3L moiety in general formula II may have the following structure:

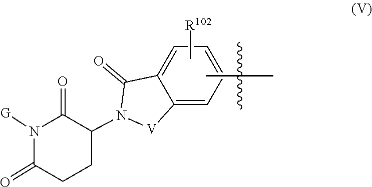

wherein R¹⁰¹, G, and V are defined as above in the present disclosure; particularly, V is selected from:

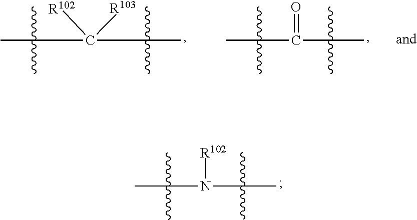

more specifically, V is selected from: —CH₂—,

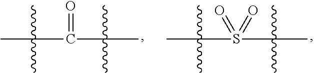

and —NH—; particularly, G is C₀₋₁₀ alkyl; more specifically, G is selected from: —H, —CH₃, and —CH₂CH₃; particularly, R¹⁰¹ is 0-3 functional groups on the ring and is selected from: —OH, halogen, —CF₃, —OCF₃, and C₁₋₁₀ alkyl.

More specifically, the E3L moiety in general formula II may have the following structure:

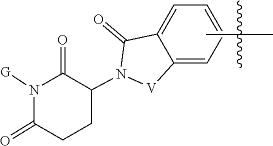

wherein G and V are defined as above in the present disclosure.

Further, the E3L moiety in general formula II may have the following structures:

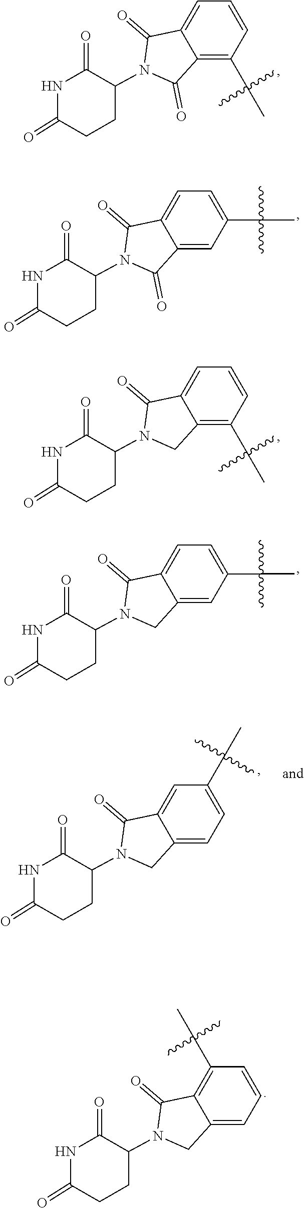

particularly

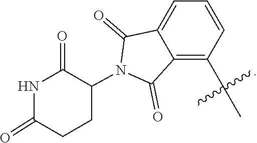 [SMILES: *c1cccc2c1C(=O)N(C1CCC(=O)NC1=O)C2=O]

In some embodiments of the present disclosure, L₃ is —N(H)—.

In some embodiments of the present disclosure, the E3 ubiquitin ligase ligand has the following structure:

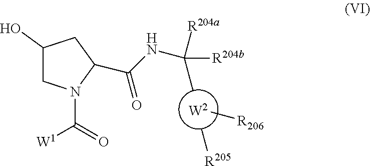

wherein W¹ is selected from aryl, heteroaryl, and

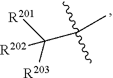

wherein R²⁰¹ and R²⁰² are independently selected from: H, alkyl, cycloalkyl, hydroxyalkyl, haloalkyl, and heteroaryl; or R²⁰¹ and R²⁰², together with the carbon atom to which they are attached, form cycloalkyl; R²⁰³ is selected from: alkyl, alkoxy, aryl, heterocyclyl, and —NR²⁰⁷R²⁰⁸; R²⁰⁷ is H or alkyl;
R²⁰⁸ is selected from: H, alkyl, alkylcarbonyl, (cycloalkyl)alkylcarbonyl, aralkylcarbonyl, arylcarbonyl, (heterocyclyl)carbonyl, and aralkyl;
W² is selected from aryl and heteroaryl;
R^{204a} and R^{204b} are independently selected from: H, alkyl, cycloalkyl, and haloalkyl;
R²⁰⁵ and R²⁰⁶ are independently selected from: H, OH, halogen, CN, NO₂, alkyl, haloalkyl, haloalkoxy, aryl, heterocyclyl, cycloalkyl, —NR^{204a}R^{204b}, —CONR^{204a}R^{204b}, —OR^{204a}R^{204b}, —NR^{204a}COR^{204b}, —SO₂NR^{204a}R^{204b}, and —NR^{204a}SO₂R^{204b}.

Specifically, the compound represented by general formula VI may have the following structure:

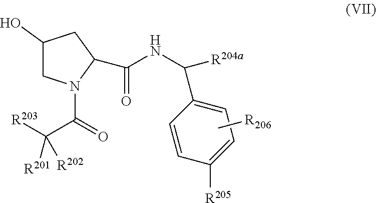

Specifically, the E3L moiety in general formula II may have the following structure:

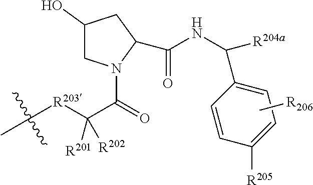

wherein R²⁰³′ is selected from: —NHCO— and 5- to 6-membered heteroarylene

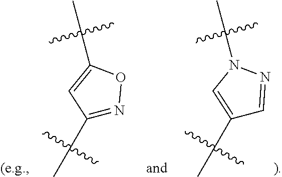 [SMILES: *c1cc(*)on1 ; *c1cnn(*)c1]

Specifically, the E3L moiety in general formula II may also have the following structure:

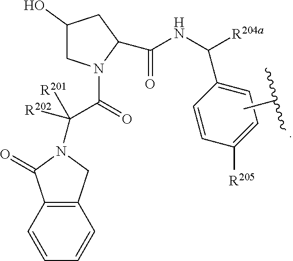

More specifically, the E3L moiety in general formula II may have the following structure:

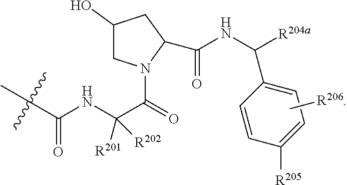

Specifically, R²⁰¹ and R²⁰² are independently selected from: C₀₋₁₀ alkyl, C₀₋₁₀ alkoxy, C₁₋₁₀ haloalkyl, and C₃₋₆ cycloalkyl; or R²⁰¹ and R²⁰², together with the carbon atom to which they are attached, form cycloalkyl; more specifically, R²⁰¹ described above is C₀₋₁₀ alkyl, such as H, methyl, ethyl, or propyl; more specifically, R²⁰² described above is C₀₋₁₀ alkyl or C₃₋₆ cycloalkyl, such as H, methyl, ethyl, isopropyl, tert-butyl, sec-butyl, cyclopropyl, cyclobutyl, cyclopentyl, or cyclohexyl.

Specifically, R^{204a} is selected from: C₀₋₁₀ alkyl, C₃₋₆ cycloalkyl, and haloalkyl; more specifically, R^{204a} is selected from: H, methyl, CF₃, CH₂F, CHF₂, hydroxymethyl, ethyl, isopropyl, and cyclopropyl.

Specifically, R²⁰⁵ is selected from: H, OH, halogen, CN, NO₂, aryl, and heteroaryl; more specifically, R²⁰⁵ described above is selected from: H, OH, halogen, CN, NO₂,

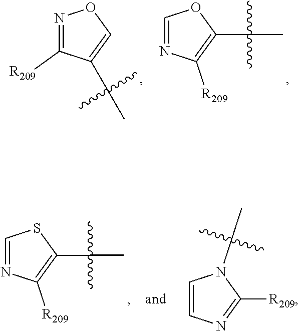

wherein R²⁰⁹ is selected from: C₀₋₁₀ alkyl, —O—C₀₋₁₀ alkyl, and haloalkyl.

Specifically, R²⁰⁹ described above is selected from: H, methyl, ethyl, propyl, hydroxy, hydroxymethyl, and hydroxyethyl.

Specifically, R²⁰⁶ is selected from: H, OH, halogen, CN, and NO₂.

More specifically, the E3L moiety in general formula II has the following structure:

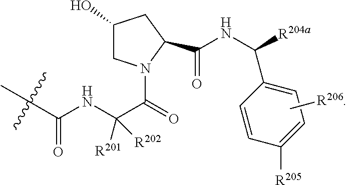

More specifically, the E3L moiety in general formula II may have the following structure:

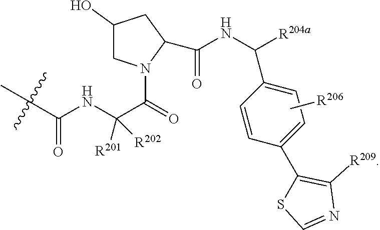

Specifically, the E3L moiety in general formula II may be selected from the following structures:

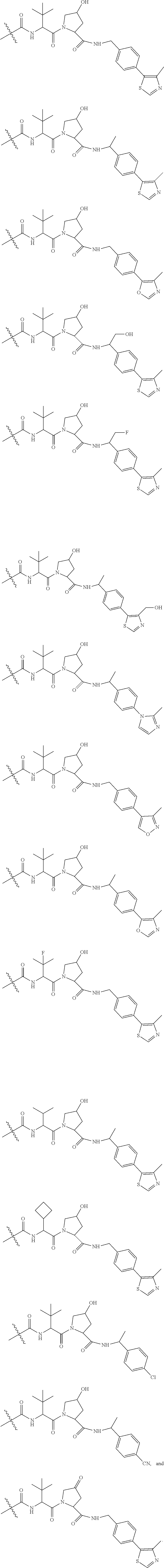

Specifically, the E3L moiety in general formula II may also be selected from the following structures:

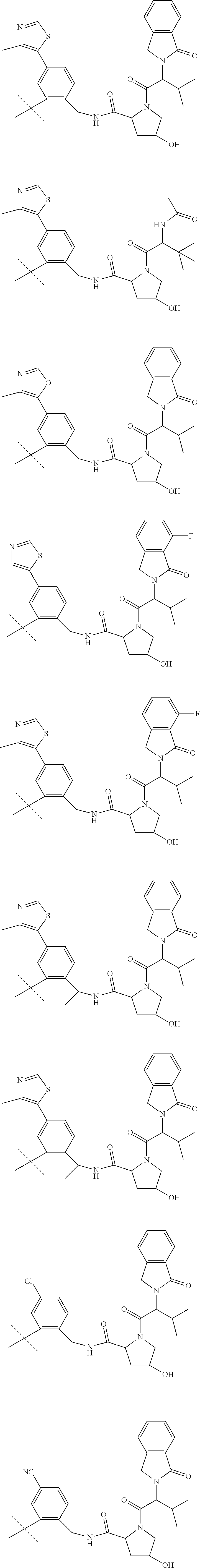

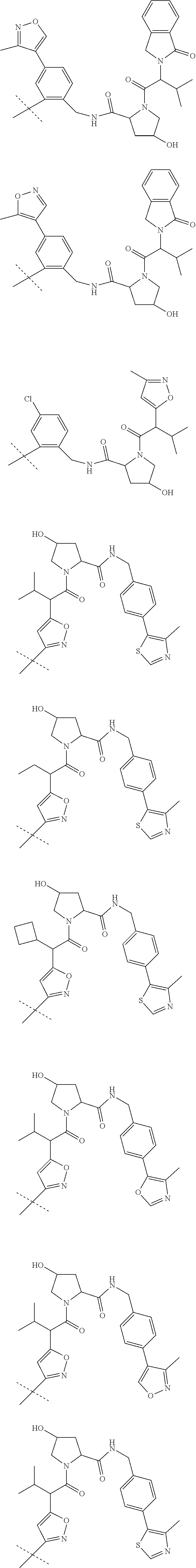

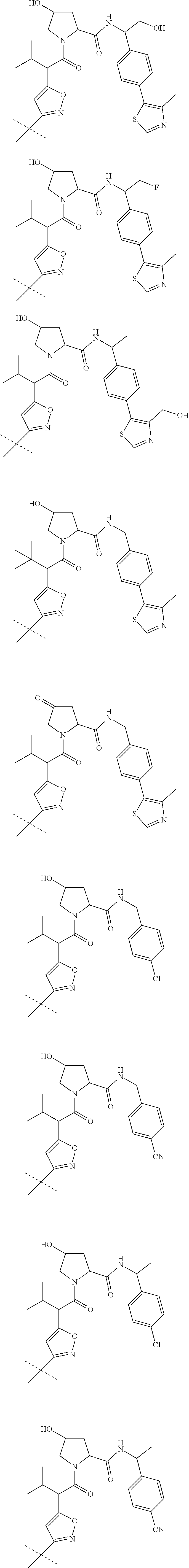

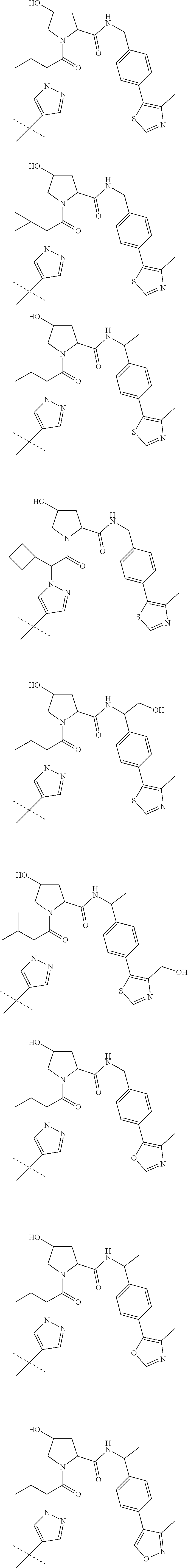

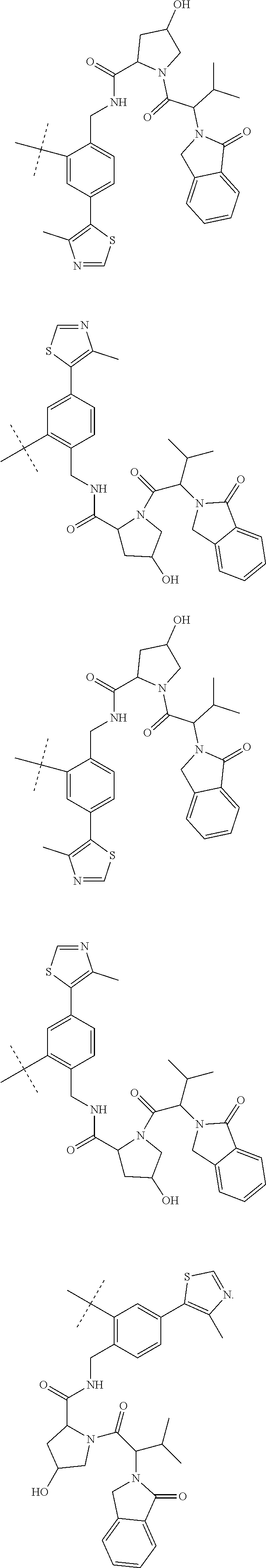

Still further, the E3L moiety in general formula II may have the following structures:

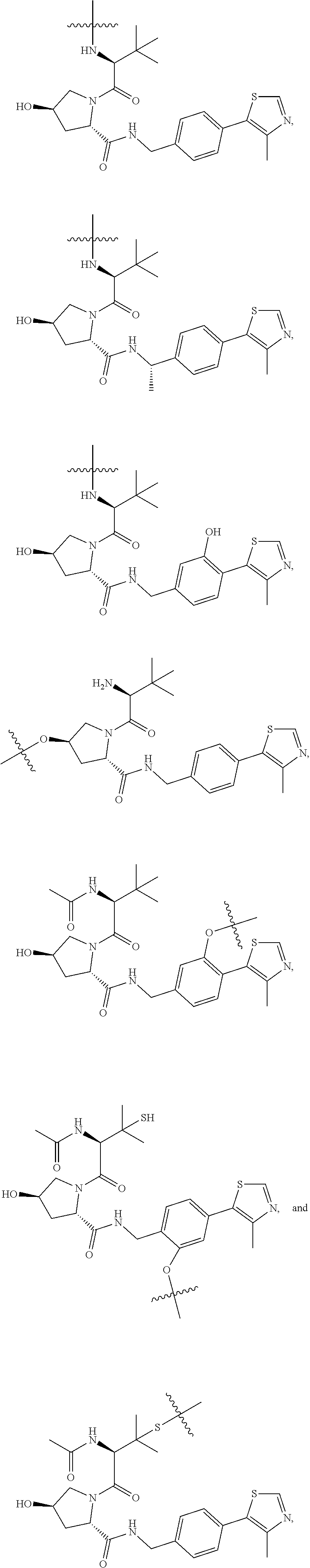

particularly

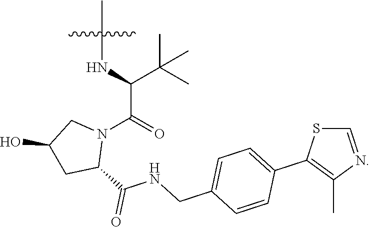 [SMILES: *N[C@H](C(=O)N1C[C@H](O)C[C@H]1C(=O)NCc1ccc(-c2scnc2C)cc1)C(C)(C)C]

In some embodiments of the present disclosure, L₃ is —C(O)—.

In some embodiments of the present disclosure, L₁ is O or a single bond, that is, the GRO is linked to L₂ via a phosphoester bond

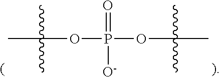 [SMILES: *OP(=O)([O-])O*]

In some embodiments of the present disclosure, the targeted protein degrader represented by general formula II has the following structure:

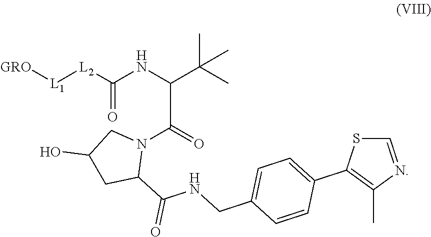

Specifically, in general formula VIII, L₂ adopts the scheme (1) and/or scheme (2) described above, particularly scheme (1).

Specifically, in general formula VIII, L₁ may be a single bond or O.

In some embodiments of the present disclosure, the targeted protein degrader represented by general formula II has the following structure:

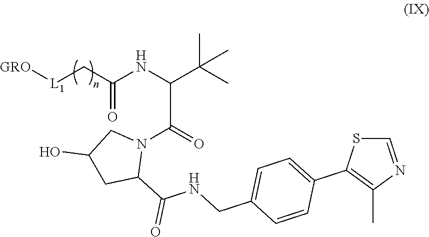

wherein n is an integer of 1-10 (e.g., 1, 2, 3, 4, 5, 6, 7, 8, 9, or 10).

In one embodiment of the present disclosure, the targeted protein degrader has the following structure:

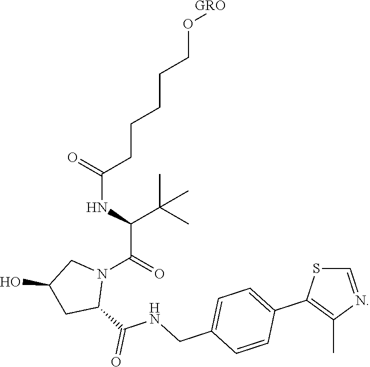

A fourth objective of the present disclosure is to provide a targeted protein degrader capable of degrading transcription factor STAT3, which is obtained by conjugating a GRO to a STAT3 protein ligand via a linker.

Specifically, the GRO is described as in the first objective of the present disclosure; in some embodiments of the present disclosure, the GRO is AS1411, GRO29A, GRO15A, or AT11.

Specifically, the linker is L described in the third objective of the present disclosure; particularly, L₁ is a single bond or O, L₂ adopts scheme (3), and L₃ is a single bond or 0; in some embodiments of the present disclosure, the linker is a single-stranded DNA linker, and preferably, the linker comprises β-30 (e.g., 3, 4, 5, 6, 7, 8, 9, 10, 12, 14, 16, 18, 20, 22, 24, 26, 28, or 30) A or T; in one embodiment of the present disclosure, the sequence of the linker is as follows: 5′-AAAAAA-3′.

Specifically, the targeted protein degrader capable of degrading transcription factor STAT3 comprises a first targeted protein degrader product conjugated to the 5′ end of the GRO and a second targeted protein degrader product conjugated to the 3′ end of the GRO.

Specifically, the STAT3 protein ligand includes small molecule compounds, oligonucleotides, oligopeptides, proteins (e.g., antibodies), and the like. In one embodiment of the present disclosure, the STAT3 protein ligand is double-stranded transcription factor decoy oligonucleotide S3 that specifically recognizes STAT3, wherein the sense strand S3-F of S3 is set forth in SEQ ID NO: 5, the antisense strand S3-R of S3 is set forth in SEQ ID NO: 6, and the sense strand or antisense strand is linked to the 5′ end or 3′ end of the GRO via a linker.

Specifically, a preparation method for the targeted protein degrader capable of degrading transcription factor STAT3 may comprise the following steps: linking the antisense strand of the double-stranded transcription factor decoy oligonucleotide S3, the single-stranded DNA linker, and the GRO together by solid-phase synthesis, then mixing with the sense strand of the double-stranded transcription factor decoy oligonucleotide S3 (e.g., in a molar ratio of 1:1), and performing base complementary pairing treatment to generate the targeted protein degrader capable of degrading transcription factor STAT3.

Specifically, the treatment comprises: heating in a metal bath, and then cooling; more specifically, the treatment comprises: heating in a metal bath at 80-100° C. for 2-10 min, and then cooling to room temperature and standing for 10-60 min; in some embodiments of the present disclosure: the treatment comprises heating in a metal bath at 95° C. for 5 min, and then cooling to room temperature and standing for 30 min.

A fifth objective of the present disclosure is to provide a targeted protein degrader capable of degrading transcription factor c-Myc, which is obtained by conjugating a GRO to a ligand of c-Myc via a linker.

Specifically, the GRO is described as in the first objective of the present disclosure; in some embodiments of the present disclosure, the GRO is AS1411, GRO29A, GRO15A, or AT11.

Specifically, the linker is L described in the third objective of the present disclosure; particularly, L₁ is a single bond or O, L₂ adopts scheme (3), and L₃ is a single bond or 0; in some embodiments of the present disclosure, the linker is a single-stranded DNA linker, and preferably, the linker comprises β-30 (e.g., 3, 4, 5, 6, 7, 8, 9, 10, 12, 14, 16, 18, 20, 22, 24, 26, 28, or 30) A or T; in one embodiment of the present disclosure, the sequence of the linker is as follows: 5′-AAAAAA-3′.

Specifically, the ligand of c-Myc includes small molecule compounds, oligonucleotides, oligopeptides, proteins (e.g., antibodies), and the like. In one embodiment of the present disclosure, the ligand of c-Myc is a double-stranded transcription factor decoy oligonucleotide, wherein the sense strand sequence MYC-F of the oligonucleotide is set forth in SEQ ID NO: 7, the antisense strand sequence MYC-R of the oligonucleotide is set forth in SEQ ID NO: 8, and the sense strand or antisense strand is linked to the 5′ end or 3′ end of the GRO via a linker.

Specifically, a preparation method for the targeted protein degrader capable of degrading transcription factor c-Myc may comprise the following steps: linking the antisense strand of the double-stranded transcription factor decoy oligonucleotide, the single-stranded DNA linker, and the GRO together by solid-phase synthesis, then mixing with the sense strand of the double-stranded transcription factor decoy oligonucleotide, and performing base complementary pairing treatment to generate the targeted protein degrader capable of degrading transcription factor c-Myc.

Specifically, the treatment comprises: heating in a metal bath, and then cooling; more specifically, the treatment comprises: heating in a metal bath at 80-100° C. for 2-10 min, and then cooling to room temperature and standing for 10-60 min; in some embodiments of the present disclosure: the treatment comprises heating in a metal bath at 95° C. for 5 min, and then cooling to room temperature and standing for 30 min.

A sixth objective of the present disclosure is to provide a targeted protein degrader capable of degrading p53-R175H, which is obtained by conjugating a GRO to a p53-R175H ligand via a linker.

Specifically, the GRO is described as in the first objective of the present disclosure; in some embodiments of the present disclosure, the GRO is AS1411, GRO29A, GRO15A, or AT11.

Specifically, the linker is L described in the third objective of the present disclosure; particularly, L₁ is a single bond or O, L₂ adopts scheme (3), and L₃ is a single bond or 0; in some embodiments of the present disclosure, the linker is a double-stranded oligonucleotide residue comprising β-30 (e.g., 3, 4, 5, 6, 7, 8, 9, 10, 12, 14, 16, 18, 20, 22, 24, 26, 28, and 30) A-T pairs; in one embodiment of the present disclosure, the linker is a double-stranded oligonucleotide consisting of 6 A-T pairs; in one embodiment of the present disclosure, the linker is a double-stranded DNA/RNA hybrid oligonucleotide, wherein the DNA single strand consists of 6 T, and the RNA single strand consists of 6 A.

Specifically, the ligand of p53-R175H includes small molecules, oligonucleotides, oligopeptides, proteins (e.g., antibodies), and the like. In one embodiment of the present disclosure, the ligand of p53-R175H is the RNA aptamer R175Hapt of p53-R175H, which has the sequence set forth in SEQ ID NO: 9 and is linked to the 5′ end or 3′ end of the GRO by a linker.

Specifically, a preparation method for the targeted protein degrader capable of degrading p53-R175H may comprise the following steps:
linking the 3′ end of the GRO to the DNA single strand of the double-stranded DNA/RNA hybrid oligonucleotide by solid-phase synthesis to form a first conjugate product;
linking the 5′ end or 3′ end of R175Hapt to the RNA single strand of the double-stranded DNA/RNA hybrid oligonucleotide by solid-phase synthesis to form a second conjugate product;
mixing the first conjugate product and the second conjugate product (e.g., in a molar ratio of 1:1), and performing A-T base complementary pairing treatment to generate the targeted protein degrader capable of degrading p53-R175H.

Specifically, the treatment comprises: separately heating the first conjugate product and the second conjugate product (e.g., in a molar ratio of 1:1) in a metal bath, and then cooling and mixing; more specifically, the treatment comprises: separately heating the first conjugate product and the second conjugate product (e.g., in a molar ratio of 1:1) in a metal bath at 80-100° C. for 2-10 min, then cooling to 35-40° C., and mixing after standing for 10-60 min; in some embodiments of the present disclosure: the treatment comprises separately heating the first conjugate product and the second conjugate product (e.g., in a molar ratio of 1:1) in a metal bath at 95° C. for 5 min, then cooling to 37° C., and mixing after standing for 30 min.

A seventh objective of the present disclosure is to provide a targeted protein degrader capable of degrading AR-V7, which is obtained by conjugating a GRO to a ligand of AR-V7 via a linker.

Specifically, the GRO is described as in the first objective of the present disclosure; in some embodiments of the present disclosure, the GRO is AS1411, GRO29A, GRO15A, or AT11.

Specifically, the linker is L described in the third objective of the present disclosure; particularly, L₁ is a single bond or O, L₂ adopts scheme (3), and L₃ is a single bond or 0; in some embodiments of the present disclosure, the linker is a double-stranded oligonucleotide residue comprising β-30 (e.g., 3, 4, 5, 6, 7, 8, 9, 10, 12, 14, 16, 18, 20, 22, 24, 26, 28, and 30) A-T pairs; in one embodiment of the present disclosure, the linker is a double-stranded oligonucleotide consisting of 6 A-T pairs; in one embodiment of the present disclosure, the linker is a double-stranded DNA/RNA hybrid oligonucleotide, wherein the DNA single strand consists of 6 T, and the RNA single strand consists of 6 A.

Specifically, the ligand of AR-V7 includes small molecules, oligonucleotides, oligopeptides, proteins (e.g., antibodies), and the like. In one embodiment of the present disclosure, the ligand of AR-V7 is RNA oligonucleotide lncRV set forth in SEQ ID NO: 10 or lncV7 set forth in SEQ ID NO: 11, which is linked to the 5′ end or 3′ end of the GRO by a linker.

Specifically, a preparation method for the targeted protein degrader capable of degrading AR-V7 may comprise the following steps:
linking the 3′ end of the GRO to the DNA single strand of the double-stranded DNA/RNA hybrid oligonucleotide by solid-phase synthesis to form a first conjugate product;
linking the 5′ end or 3′ end of lncV7 to the RNA single strand of the double-stranded DNA/RNA hybrid oligonucleotide by solid-phase synthesis to form a second conjugate product;
mixing the first conjugate product and the second conjugate product (e.g., in a molar ratio of 1:1), and performing A-T base complementary pairing treatment to generate the targeted protein degrader capable of degrading AR-V7.

Specifically, the treatment comprises: separately heating the first conjugate product and the second conjugate product (e.g., in a molar ratio of 1:1) in a metal bath, and then cooling and mixing; more specifically, the treatment comprises: separately heating the first conjugate product and the second conjugate product (e.g., in a molar ratio of 1:1) in a metal bath at 80-100° C. for 2-10 min, then cooling to 35-40° C., and mixing after standing for 10-60 min; in some embodiments of the present disclosure: the treatment comprises separately heating the first conjugate product and the second conjugate product (e.g., in a molar ratio of 1:1) in a metal bath at 95° C. for 5 min, then cooling to 37° C., and mixing after standing for 30 min.

An eighth objective of the present disclosure is to provide a targeted protein degrader capable of degrading VEGF165, which is obtained by conjugating a GRO to a ligand of VEGF165 via a linker.

Specifically, the GRO is described as in the first objective of the present disclosure; in some embodiments of the present disclosure, the GRO is AS1411, GRO29A, GRO15A, or AT11.

Specifically, the linker is L described in the third objective of the present disclosure; particularly, L₁ is a single bond or O, L₂ adopts scheme (3), and L₃ is a single bond or 0; in some embodiments of the present disclosure, the linker is a single-stranded DNA linker, and preferably, the linker comprises β-30 (e.g., 3, 4, 5, 6, 7, 8, 9, 10, 12, 14, 16, 18, 20, 22, 24, 26, 28, or 30) A or T; in one embodiment of the present disclosure, the sequence of the linker is as follows: 5′-AAAAAA-3′.

Specifically, the ligand of VEGF165 includes small molecule compounds, oligonucleotides, oligopeptides, proteins (e.g., antibodies), and the like. In one embodiment of the present disclosure, the ligand of VEGF165 is single-stranded DNA aptamer V7t1, which has the sequence set forth in SEQ ID NO: 12 and is linked to the 5′ end or 3′ end of the GRO by a linker.

Specifically, a preparation method for the targeted protein degrader capable of degrading VEGF165 may comprise the following step: linking the DNA aptamer V7t1 of VEGF165, the single-stranded DNA linker, and the GRO together by solid-phase synthesis to generate the targeted protein degrader capable of degrading VEGF165.

A ninth objective of the present disclosure is to provide a targeted protein degrader capable of degrading MDM2, which is obtained by conjugating a GRO to a ligand of an E3 ubiquitin ligase via a linker.

Specifically, the GRO is described as in the first objective of the present disclosure; in some embodiments of the present disclosure, the GRO is AS1411, GRO29A, GRO15A, or AT11.

Specifically, the linker is L described in the third objective of the present disclosure; particularly, L₁ is a single bond or O, and L₂ adopts scheme (1) or (2); in one embodiment of the present disclosure, L₂ is C1-C20 linear alkylene.

Specifically, the E3 ubiquitin ligase ligand includes small molecule compounds, oligonucleotides, oligopeptides, and proteins (e.g., antibodies); preferably, the oligonucleotides include nucleic acid aptamers and the like. In one embodiment of the present disclosure, the E3 ubiquitin ligase is VHL, and the E3 ubiquitin ligase ligand is small molecule VH032 linked to the 5′ end or 3′ end of the GRO by a linker.

In some embodiments of the present disclosure, the targeted protein degrader capable of degrading MDM2 has the structure of formula VIII described above, particularly the structure of formula IX, of the present disclosure.

A tenth objective of the present disclosure is to provide a homo-targeted protein degrader (homo-PROTAC) capable of degrading MDM2, which is formed by conjugating a first GRO to a second GRO via a linker. Therefore, the GROs serve not only as the recruitment element of a target protein in the homo-targeted protein degrader but also as the recruitment element of an E3 ubiquitin ligase, and can simultaneously recruit MDM2 at both ends of the targeted protein degrader molecule for “suicide” degradation, thereby achieving the treatment of MDM2-associated diseases.

Specifically, the first GRO is described as in the first objective of the present disclosure; in one embodiment of the present disclosure, the first GRO is AS1411.

Specifically, the second GRO is described as in the first objective of the present disclosure; in one embodiment of the present disclosure, the second GRO is AS1411.

Specifically, the linker is L described in the third objective of the present disclosure; particularly, L₁ is a single bond or O, L₂ adopts scheme (3), and L₃ is a single bond or 0; in some embodiments of the present disclosure, the linker is a double-stranded oligonucleotide residue comprising β-30 (e.g., 3, 4, 5, 6, 7, 8, 9, 10, 12, 14, 16, 18, 20, 22, 24, 26, 28, and 30) A-T pairs; in one embodiment of the present disclosure, the linker is a double-stranded DNA oligonucleotide consisting of 6 A-T pairs.

Specifically, a preparation method for the homo-targeted protein degrader capable of degrading MDM2 may comprise the following steps:
linking the 3′ end of the first GRO to the single strand with 6 A bases of the double-stranded DNA oligonucleotide by solid-phase synthesis to form a first conjugate product;
linking the 3′ end of the second GRO to the single strand with 6 T bases of the double-stranded DNA oligonucleotide by solid-phase synthesis to form a second conjugate product;
mixing the first conjugate product and the second conjugate product (e.g., in a molar ratio of 1:1), and performing A-T base complementary pairing treatment to generate the homo-targeted protein degrader capable of degrading MDM2.

Specifically, the treatment comprises: separately heating the first conjugate product and the second conjugate product (e.g., in a molar ratio of 1:1) in a metal bath, and then cooling and mixing; more specifically, the treatment comprises: separately heating the first conjugate product and the second conjugate product (e.g., in a molar ratio of 1:1) in a metal bath at 80-100° C. for 2-10 min, then cooling to 35-40° C., and mixing after standing for 10-60 min; in some embodiments of the present disclosure: the treatment comprises separately heating the first conjugate product and the second conjugate product (e.g., in a molar ratio of 1:1) in a metal bath at 95° C. for 5 min, then cooling to 37° C., and mixing after standing for 30 min.

An eleventh objective of the present disclosure is to provide a targeted protein degrader capable of degrading c-MET, which is obtained by conjugating a GRO to a c-MET ligand via a linker.

Specifically, the GRO is described as in the first objective of the present disclosure; in some embodiments of the present disclosure, the GRO is AS1411, GRO29A, GRO15A, or AT11.

Specifically, the linker is L described in the third objective of the present disclosure.

Specifically, the c-MET ligand includes small molecules, oligonucleotides, oligopeptides, proteins (e.g., antibodies), and the like.

In some embodiments of the present disclosure, L₁ is a single bond or O, L₂ adopts scheme (3), and L₃ is a single bond or O; in some embodiments of the present disclosure, the linker is a double-stranded oligonucleotide residue comprising 3-30 (e.g., 3, 4, 5, 6, 7, 8, 9, 10, 12, 14, 16, 18, 20, 22, 24, 26, 28, and 30) A-T pairs; in one embodiment of the present disclosure, the linker is a double-stranded oligonucleotide consisting of 6 A-T pairs; in one embodiment of the present disclosure, the linker is a double-stranded oligonucleotide consisting of 10 A-T pairs.

In one embodiment of the present disclosure, the c-MET ligand is DNA aptamer SL1 having the sequence set forth in SEQ ID NO: 19.

Specifically, a preparation method for the targeted protein degrader capable of degrading c-MET may comprise the following steps:
linking the 3′ end of the GRO to the single strand with 6 T bases of the double-stranded DNA oligonucleotide by solid-phase synthesis to form a first conjugate product;
linking the 5′ end or 3′ end of SL1 to the single strand with 6 A bases of the double-stranded DNA oligonucleotide by solid-phase synthesis to form a second conjugate product;
mixing the first conjugate product and the second conjugate product (e.g., in a molar ratio of 1:1), and performing A-T base complementary pairing treatment to generate the targeted protein degrader capable of degrading c-MET.

Specifically, the treatment comprises: separately heating the first conjugate product and the second conjugate product (e.g., in a molar ratio of 1:1) in a metal bath, and then cooling and mixing; more specifically, the treatment comprises: separately heating the first conjugate product and the second conjugate product (e.g., in a molar ratio of 1:1) in a metal bath at 80-100° C. for 2-10 min, then cooling to 35-40° C., and mixing after standing for 10-60 min; in some embodiments of the present disclosure: the treatment comprises separately heating the first conjugate product and the second conjugate product (e.g., in a molar ratio of 1:1) in a metal bath at 95° C. for 5 min, then cooling to 37° C., and mixing after standing for 30 min.

In some embodiments of the present disclosure, L₁ is a single bond or 0, and L₂ adopts scheme (1) and/or scheme (2).

In one embodiment of the present disclosure, the c-MET ligand is tepotinib, which is linked to the 5′ end or 3′ end of the GRO by a linker.

A twelfth objective of the present disclosure is to provide a pharmaceutical composition for treating a related disease by degrading a target protein, and the pharmaceutical composition comprises the targeted protein degrader described above, and one or more pharmaceutically acceptable auxiliary materials.

Specifically, the pharmaceutically acceptable auxiliary materials may be selected from: one or more of fillers, binders, lubricants, disintegrants, antioxidants, buffers, bacteriostatic agents, suspending agents, solubilizers, thickeners, stabilizers, preservatives, and the like.

Specifically, the pharmaceutical composition may be administered by any suitable route of administration, such as gastrointestinal routes (e.g., oral, sublingual, and rectal administration) or parenteral routes (e.g., intravenous, intramuscular, intranasal, intraocular, intracerebral, intravaginal, intraperitoneal, transdermal, subcutaneous, intradermal, and respiratory administration).

Specifically, the pharmaceutical composition may be prepared as pharmaceutical formulations in the form of: injections, syrups, elixirs, suspensions, powders, granules, tablets, capsules, lozenges, creams, ointments, lotions, gels, emulsions, and the like.

In the preparation of injections, any carrier commonly used in the art can be used, for example, water, ethanol, propylene glycol, ethoxylated isostearyl alcohol, polyethoxylated isostearyl alcohol, fatty acid esters of polyethylene sorbitan, and the like. In addition, conventional solvents and buffers can be added.

Specifically, the pharmaceutical composition is preferably in unit dosage form. In this form, the formulation is subdivided into unit dosages containing an appropriate amount of active ingredients. The unit dosage form can be capsules, tablets, or any other dosage forms; in addition, the unit dosage form can be packaged formulations such as tablets, capsules, and powders packaged in vials or ampoules.

Specifically, in the pharmaceutical composition, the targeted protein degrader may be used alone or in combination with other kinds of active ingredients.

Specifically, the amount of the active ingredients in the unit dosage formulation may be varied or adjusted from 0.1 mg to 1000 mg (e.g., 0.1 mg, 1 mg, 5 mg, 10 mg, 20 mg, 40 mg, 50 mg, 100 mg, 200 mg, 400 mg, 500 mg, or 1000 mg), depending on the specific application and the potency of the active ingredients. The composition may further contain other suitable therapeutic agents, if desired.

Preferably, the disease is a disease that can be prevented and/or treated beneficially by inhibiting/degrading the target protein; further preferably, the disease is at least one of a tumor, an autoimmune disease, an inflammatory disease, a disease associated with pathogen infection, a neurodegenerative disease, a cardiovascular disease, a metabolic disease, a fibrotic disease, and an ophthalmic disease.

More preferably, the tumor is selected from: lung cancer, malignant melanoma, brain tumor, tumors of digestive organs, uterine cancer, testicular cancer, cancer of the upper jaw, throat cancer, tongue cancer, oral cancer, various sarcomas, osteosarcoma, hematological tumors, tumors of nervous system, brain glioma, glioblastoma, skin cancer, skin appendage carcinoma and metastatic carcinoma of skin, medulloblastoma, blastoma, liposarcoma, neuroendocrine tumors, synovial cell sarcoma, gastrinoma, carcinoid tumors, mesothelioma, islet cell carcinoma, schwannoma, meningioma, melanoma, acoustic neuroma, adenocarcinoma, lymphoid malignancy, epithelial squamous cell carcinoma, small cell lung carcinoma, non-small cell lung carcinoma, squamous cell carcinoma, adenocarcinoma lung carcinoma, peritoneal carcinoma, lung squamous cell carcinoma, hepatocellular carcinoma, gastric cancer, intestinal cancer, pancreatic cancer, glioblastoma, cervical cancer, ovarian cancer, liver cancer, thyroid cancer, bladder cancer, breast cancer, metastatic breast cancer, colon cancer, rectal cancer, prostate cancer, salivary gland cancer, renal cancer, vulvar cancer, anal cancer, penile cancer, esophageal cancer, biliary tract tumors, and head and neck cancer.

More preferably, the autoimmune disease is selected from: organ-specific autoimmune disease, systemic lupus erythematosus, rheumatoid arthritis, systemic vasculitis, scleroderma, pemphigus, dermatomyositis, mixed connective tissue disease, autoimmune hemolytic anemia, thyroid autoimmune disease, and ulcerative colitis.

More preferably, the inflammatory disease is selected from: osteoarthritis, acute gout, multiple sclerosis, inflammatory bowel disease (e.g., Crohn's disease and ulcerative colitis), neuroinflammation, asthma, chronic obstructive airway disease, pneumonia, myositis, eczema, dermatitis, acne, cellulitis, occlusive disease, thrombosis, alopecia, nephritis, vasculitis, retinitis, uveitis, scleritis, sclerosing cholangitis, hypophysitis, thyroiditis, septic shock, systemic inflammatory response syndrome (SIRS), toxic shock syndrome, acute lung injury, ARDS (adult respiratory distress syndrome), acute renal failure, burns, pancreatitis (e.g., acute pancreatitis), post-operative syndrome, sarcoidosis, Herxheimer reaction, encephalitis, myelitis, meningitis, and malaria.

More preferably, the neurodegenerative disease is selected from: Alzheimer's disease (AD), Parkinson's disease (PD), Huntington's disease (HD), amyotrophic lateral sclerosis (ALS), different types of spinocerebellar ataxia (SCA), and Pick's disease.

More preferably, the disease associated with pathogen infection is selected from: influenza, SARS, COVID-19, viral hepatitis (e.g., hepatitis A, hepatitis B, hepatitis C, hepatitis D, etc.), AIDS, rabies, Dengue fever, and Ebola virus disease.

More preferably, the cardiovascular disease is selected from: coronary heart disease, peripheral arterial disease, atherosclerosis, ischemic heart disease, ischemic cardiomyopathy, myocardial infarction, heart failure, angina pectoris, myocarditis, hypercholesterolemia, hypertensive disease, ischemia-reperfusion injury, cerebrovascular ischemia (stroke), embolism (e.g., pulmonary embolism, renal embolism, hepatic embolism, gastrointestinal embolism, or peripheral limb embolism), and myocardial ischemia.

More preferably, the metabolic disease is selected from: diabetes (e.g., type I diabetes, type II diabetes, or gestational diabetes), obesity, fatty liver (NASH or other), cachexia, hypercholesterolemia, and gout.

More preferably, the fibrotic disease is selected from: myocardial fibrosis, pulmonary fibrosis, renal fibrosis, postsurgical stenosis, keloid formation, cirrhosis, biliary cirrhosis, and scleroderma.

More preferably, the ophthalmic disease is selected from: proliferative diabetic retinopathy, diabetic macular edema, herpes simplex virus stromal keratitis, age-related macular degeneration, uveitis, rubeosis iridis, conjunctivitis, keratitis, blepharitis marginalis, hordeolum, chalazion, iritis, macular degeneration, retinopathy, and the like.

A thirteenth objective of the present disclosure is to provide a delivery system for a targeted protein degrader, which comprises the targeted protein degrader described above and a carrier and can deliver the targeted protein degrader of the present disclosure to a target cell or a target tissue without NCL expression on the cell surface.

Specifically, the carrier described above may be any carrier suitable for delivering the nucleic acid drug to a target tissue or a target cell, such as those disclosed in the prior art (see, e.g., Wang Junfeng, Tan Manman, Wang Ying et al., Advances in Modification and Delivery of Nucleic Acid Drugs [J]. Journal of Zhejiang University (Medical Edition), 2023, 52(04): 417-428, which is incorporated herein by reference), such as viral vectors (e.g., lentivirus, adenovirus, and adeno-associated viral vectors) and non-viral carriers (e.g., lipid nanoparticles (LNPs), polymeric nanocarriers, inorganic nanocarriers, protein carriers, exosomes, etc.).

In some embodiments of the present disclosure, the carrier is a lipid nanoparticle (LNP).

A fourteenth objective of the present disclosure is to provide use of the targeted protein degrader, the pharmaceutical composition, and the delivery system described above in the preparation of a medicament for preventing and/or treating a disease.

Specifically, the disease is described as in the twelfth objective of the present disclosure.

In some embodiments of the present disclosure, the targeted protein degrader has the structure of general formula I described above, and the disease is a disease associated with the target protein.

In one embodiment of the present disclosure, the target protein is STAT3, and the disease is selected from: a tumor, an autoimmune disease, an inflammatory disease, a diseases associated with pathogen infection, a cardiovascular disease, and a metabolic disease; more specifically, the disease is selected from: (ulcerative) colitis, rheumatoid arthritis, ovarian cancer, prostate cancer, pancreatic cancer, melanoma, colon cancer, bladder cancer, cervical cancer, psoriasis, diabetes, asthma, and the like.

In one embodiment of the present disclosure, the target protein is Myc (e.g., c-Myc), and the disease is a tumor, such as lung cancer, gastric cancer, breast cancer, colon cancer, cervical cancer, myeloid leukemia, retinoblastoma, osteosarcoma, chondrosarcoma, chordoma, liposarcoma, rhabdomyosarcoma, Hodgkin's disease, and head tumor.

In one embodiment of the present disclosure, the target protein is a p53 mutant (e.g., p53-R175H, G245S, R248W/Q, R249S, R273C/H, and R282W), and the disease is a tumor, such as liver cancer, breast cancer, bladder cancer, gastric cancer, colon cancer, prostate cancer, soft tissue sarcoma, ovarian cancer, brain tumor, lymphocytic tumor, cancer of esophagus, lung cancer, and osteosarcoma.

In one embodiment of the present disclosure, the target protein is a splice mutant of AR (e.g., AR-V7), and the disease is a tumor, such as prostate cancer, particularly (metastatic) castration-resistant prostate cancer.

In one embodiment of the present disclosure, the target protein is VEGF (such as VEGF-A (e.g., VEGF121, VEGF145, VEGF148, VEGF183, VEGF165, VEGF189, and VEGF206), VEGF-B, VEGF-C, VEGF-D, and VEGF-E), and the disease is selected from: a tumor (such as lung cancer (e.g., non-small cell lung cancer), liver cancer, esophageal cancer, leukemia, cervical cancer, colorectal cancer, pancreatic cancer, renal cancer, bladder cancer, breast cancer, prostate cancer, gastric cancer, oral epithelial cancer, ovarian cancer, head and neck cancer, brain tumor, and glioblastoma), an ophthalmic disease (e.g., proliferative diabetic retinopathy, diabetic macular edema, herpes simplex virus stromal keratitis, age-related macular degeneration, uveitis, rubeosis iridis, conjunctivitis, keratitis, blepharitis marginalis, hordeolum, chalazion, iritis, macular degeneration, retinopathy, etc.), and a cardiovascular disease (e.g., atherosclerosis, etc.).

In one embodiment of the present disclosure, the target protein is MDM2, and the disease is selected from: a tumor, an autoimmune disease, an inflammatory disease, and a disease associated with pathogen infection, such as acute monocytic leukemia, acute myelogenous leukemia, chronic myelogenous leukemia, chronic lymphocytic leukemia, mixed lineage leukemia, nuclear protein in testis (NUT) carcinoma, multiple myeloma, small cell lung cancer (SCLC), neuroblastoma, Burkitt's lymphoma, cervical cancer, cancer of esophagus, ovarian cancer, colorectal cancer, prostate cancer, and breast cancer.

In one embodiment of the present disclosure, the target protein is c-MET, and the disease is a tumor, such as lung cancer (e.g., non-small cell lung cancer and lung adenocarcinoma), gastric cancer, colon cancer, rectal cancer, glioblastoma, pancreatic cancer, head and neck cancer, breast cancer, ovarian cancer, liver cancer, renal cancer, bladder cancer, prostate cancer, brain cancer, uterine cancer, skin cancer, thyroid cancer, myeloma, melanoma, lymphoma, adrenocortical carcinoma, acute myelogenous leukemia, chronic myelogenous leukemia, acute lymphocytic leukemia, and chronic lymphocytic leukemia; the tumor may be a primary tumor or a metastatic tumor.

In some embodiments of the present disclosure, the targeted protein degrader has the structure of general formula II described above, and the disease is a disease associated with MDM2, as described above.

A fifteenth objective of the present disclosure is to provide a method for preventing and/or treating a disease, which comprises a step of administering to a subject in need thereof an effective amount of the targeted protein degrader, the pharmaceutical composition, or the delivery system described above.

Specifically, the disease is described as in the eleventh objective of the present disclosure; more specifically, the targeted protein degrader and the corresponding disease are described as in the fourteenth objective of the present disclosure.

Specifically, the subject is a mammal, particularly a human.

Specifically, the administration may be performed by any suitable route of administration, such as gastrointestinal routes (e.g., oral, sublingual, and rectal administration) or parenteral routes (e.g., intravenous, intramuscular, intranasal, intraocular, intracerebral, intravaginal, intraperitoneal, transdermal, subcutaneous, intradermal, and respiratory administration).

The present disclosure also provides a method for preparing a targeted protein degrader, which comprises a step of preparing a GRO and/or reactively linking the GRO to a linker (by, for example, solid-phase synthesis).

Specifically, the method further comprises a step of reactively linking the linker to a target protein ligand.

Specifically, the targeted protein degrader is described as in the first objective of the present disclosure; in some preferred embodiments of the present disclosure, the drug that degrades the target protein is a PROTAC drug.

Specifically, the GRO is described as in the first objective of the present disclosure; in some embodiments of the present disclosure, the GRO is selected from: AS1411, GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M having the nucleotide sequences set forth in SEQ ID NOs: 1-4 and 20-47, respectively; particularly, the GRO is selected from AS1411, GRO29A, GRO15A, and AT11.

Specifically, the target protein ligand is described as in the second and third objectives of the present disclosure.

Compared with the prior art, the technical solutions of the present disclosure have the following beneficial effects: The present disclosure relates to use of GROs as a recruitment element of MDM2 in a drug for degrading a target protein, and provides a targeted protein degrader constructed based on GROs, a novel recruitment element of MDM2.

GROs, as a novel recruitment element of MDM2, endow the targeted protein degrader with targeting and penetration effects on diseased cells (such as tumors), reduce the non-specific distribution of the targeted protein degrader in normal tissues, reduce the toxic and side effects of the targeted protein degrader, and improve the ability and efficiency of the targeted protein degrader to enter diseased cells (such as tumors) through the multifunctional shuttle protein characteristics of NCL and the characteristics of increased expression level in various diseased cells (such as tumors). The targeted protein degrader constructed based on GROs, a novel recruitment element of MDM2, can achieve the targeting effect on diseased cells (such as tumors) of targeted protein degraders without additionally introducing other target heads that recognize diseased cells (such as tumors). Compared with targeted protein degraders that require additional introduction of other target heads that recognize diseased cells (such as tumors), the targeted protein degrader is more ingenious and simple, which is conducive to synthesis and mass production. As a novel recruitment element of MDM2, GROs can be used to construct macromolecular targeted protein degraders, so as to avoid the problem that some difficult-to-drug target proteins do not have small molecule ligands and achieve cell penetration without any auxiliary means, thereby improving the application prospect of macromolecular targeted protein degraders.

According to the present disclosure, GROs are used as a novel recruitment element for recruiting MDM2 in targeted protein degraders, which is beyond the previous understanding and use of GROs and belongs to the category of drug redirection. GROs have wide clinical application prospects. Compared with the previously commonly used ligand Nutlin-3a of MDM2 and derivatives thereof, GROs are used as a novel recruitment element for recruiting MDM2 in targeted protein degraders and have the characteristics of low immunogenicity, good thermal stability, excellent abilities to penetrate and target diseased cells (such as tumors), good resistance to serum degradation, and the like, and the in vivo application safety of GROs is high.

### BRIEF DESCRIPTION OF THE DRAWINGS

The present disclosure will be further illustrated with reference to the drawings and examples. Among the drawings:

FIG. 1A is a graph showing the results that NCL binds to MDM2 and that AS1411 does not affect the interaction between NCL and MDM2 in Example 1;

FIG. 1B is a graph showing the results that NCL binds to MDM2 and that iSN04 blocks the interaction between NCL and MDM2 in Example 1;

FIG. 2 is a graph showing the results that AS1411 is a GRO having stable G4 structural characteristics in Example 1;

FIG. 3 is a graph showing the results that iSN04 exhibits weak G4 structural characteristics in Example 1;

FIG. 4A is a graph showing the results that the CRO sequence is unable to capture NCL and MDM2 in Example 1;

FIG. 4B is a graph showing the results that AS1411 captures NCL and MDM2 in a concentration-dependent manner in Example 1;

FIG. 4C is a graph showing the results that iSN04 can capture NCL only at a high concentration but cannot capture MDM2 in Example 1;

FIG. 5A is a graph showing the results that NCL binds to MDM2 in Example 1;

FIG. 5B is a graph showing the results that the CRO cannot bind to either NCL or MDM2 in Example 1;

FIG. 5C is a graph showing the results that AS1411 recruits MDM2 in large amounts depending on its interaction with NCL in Example 1;

FIG. 5D shows that iSN04 can only bind to NCL but cannot recruit MDM2 depending on its interaction with NCL in Example 1;

FIG. 6 is a graph showing the results for the binding of the CRO and AS1411 to HeLa cervical cancer cells in Example 2;

FIG. 7 is a graph showing the results that AS1411 recruits MDM2 depending on its interaction with NCL in Example 2;

FIG. 8 is a graph showing the results that the CRO or AS1411 penetrates HeLa cells and colocalizes with NCL in Example 2;

FIG. 9 is a graph showing the results for the intracellular colocalization of the CRO or AS1411 with MDM2 in Example 2;

FIG. 10 is a graph showing the results for the intracellular colocalization of AS1411 with MDM2 when NCL is silenced in Example 2;

FIG. 11 is a schematic diagram showing a possible mode of action of the PROTAC formed by using a GRO (e.g., AS1411) as an E3 ubiquitin ligase recruitment element in Example 2;

FIG. 12 is a schematic diagram showing a possible mode of action of the PROTAC formed by using a GRO (e.g., AS1411) as a target protein recruitment element in Example 2;

FIG. 13 shows the toxicity of AS1411 or the small molecule ligand nutlin-3a of MDM2 to MCF 10A human normal mammary epithelial cells in Example 2;

FIG. 14 is a graph showing the results for the protein degradation of c-MET in HOS osteosarcoma cells by AS1411-Tep at different concentrations and time points in Example 3;

FIG. 15A is a graph showing the results for the protein degradation of c-MET in HOS osteosarcoma cells by iSN04-Tep at different concentrations in Example 3;

FIG. 15B is a graph showing the results for the protein degradation of c-MET in HOS osteosarcoma cells by AS1411-SL1 in different formats in Example 3;

FIG. 16A is a schematic diagram showing the PROTAC products of the preferred Example 4 of the present disclosure, including a first PROTAC product 1411-S3-1 and a second PROTAC product 1411-S3-2;

FIG. 16B is an electropherogram showing the purity and size of the first PROTAC product 1411-S3-1 and the second PROTAC product 1411-S3-2 in Example 4;

FIG. 17 is a graph showing the results for the serum stability of 1411-S3-1 and 1411-S3-2 in Example 4;

FIG. 18 is a graph showing the results for the protein degradation abilities of 1411-S3-1 and 1411-S3-2 to STAT3 in HeLa cervical cancer cells at different time points in Example 4;

FIG. 19 is a graph showing the results for the protein degradation abilities of 1411-S3-1 and 1411-S3-2 to STAT3 in HeLa cervical cancer cells at different concentrations in Example 4;

FIG. 20 is a graph showing the results for comparing the protein degradation abilities of 1411-S3-1, 1411-S3-2, S3, AS1411, and AS1411+S3 to STAT3 in HeLa cervical cancer cells in Example 4;

FIG. 21 is a graph showing the results for the ubiquitination of STAT3 in HeLa cervical cancer cells by 1411-S3-1 and 1411-S3-2 in Example 4;

FIG. 22 is a graph showing the results for the inhibition of the STAT3 protein degradation mediated by 1411-S3-1 and 1411-S3-2 in HeLa cervical cancer cells by MG132 in Example 4;

FIG. 23 is a graph showing the results for the effects of silencing NCL and MDM2 on the STAT3 protein degradation mediated by 1411-S3-1 and 1411-S3-2 in HeLa cervical cancer cells in Example 4;

FIG. 24A is a graph showing the results for the protein degradation of STAT3 in HOS osteosarcoma cells by 1411-S3-1 and 1411-S3-2 in Example 4;

FIG. 24B is a graph showing the results for the protein degradation of STAT3 in MCF 10A human normal mammary epithelial cells by 1411-S3-1 and 1411-S3-2 in Example 4;

FIG. 24C is a graph showing the results for the protein degradation of STAT3 in MCF 10A human normal mammary epithelial cells by liposome-encapsulated 1411-S3-1 and 1411-S3-2 in Example 4;

FIGS. 25A-25C are graphs showing the results for the effects of 1411-S3-1 and 1411-S3-2 on the colony formation, cell proliferation, and cell apoptosis of HeLa cells in Example 5, respectively;

FIG. 26 is a graph showing the targeting effect of 1411-S3-2 on tumor tissues in vivo in Example 6;

FIGS. 27A-27C are graphs showing the results for the in vivo antitumor effect and safety of 1411-S3-2 in Example 7, respectively;

FIG. 28A is a graph showing the results for the protein degradation abilities of GRO29A-S3-1 and GRO29A-S3-2 to STAT3 in HeLa cervical cancer cells at different concentrations in Example 8;

FIG. 28B is a graph showing the results for the protein degradation abilities of GRO15A-S3-1 and GRO15A-S3-2 to STAT3 in HeLa cervical cancer cells at different concentrations in Example 9;

FIG. 29 is a graph showing the results for the protein degradation abilities of AT11-S3-1 and AT11-S3-2 to STAT3 in HeLa cervical cancer cells at different concentrations in Example 10;

FIG. 30 is a schematic diagram showing the PROTAC products in Example 11, including a first PROTAC product 1411-MYC-1 and a second PROTAC product 1411-MYC-2;

FIG. 31 is a graph showing the results for western blot assay for protein degradation in Example 11;

FIG. 32 is a graph showing the results for western blot assay after using proteasome inhibitor MG132 in Example 11;

FIG. 33 is a graph showing the results for western blot assay after using si-NCL to silence NCL in Example 11;

FIG. 34 is a graph showing the results for western blot assay after using si-MDM2 to silence MDM2 in Example 11;

FIG. 35 is a graph showing the results for the colony formation experiment in Example 11;

FIG. 36 is a graph showing the results for the cell apoptosis experiment in Example 11;

FIG. 37 is a schematic diagram showing the PROTAC products in Example 12, including AS1411-175-1 or AS1411-175-2;

FIG. 38 is a graph showing the results for western blot assay for protein degradation in Example 12;

FIG. 39 is a graph showing the results for western blot assay after using proteasome inhibitor MG132 in Example 12;

FIG. 40 is a graph showing the results for western blot assay after using si-NCL to silence NCL in Example 12;

FIG. 41 is a graph showing the results for western blot assay after using si-MDM2 to silence MDM2 in Example 12;

FIG. 42 is a graph showing the results for the colony formation experiment in Example 12;

FIG. 43 is a graph showing the results for the cell apoptosis experiment in Example 12;

FIG. 44 is a schematic diagram showing the PROTAC products in Example 13, including AS1411-V7-1 or AS1411-V7-2;

FIG. 45 is a graph showing the results for western blot assay for protein degradation in Example 13;

FIG. 46 is a graph showing the results for western blot assay after using proteasome inhibitor MG132 in Example 13;

FIG. 47 is a graph showing the results for western blot assay after using si-NCL to silence NCL in Example 13;

FIG. 48 is a graph showing the results for western blot assay after si-MDM2 silences MDM2 in Example 13;

FIG. 49 is a graph showing the results for the colony formation experiment in Example 13;

FIG. 50 is a graph showing the results for the cell apoptosis experiment in Example 13;

FIG. 51 is a graph showing the results for the VEGF165 protein degradation by 1411-V7t1-1 and 1411-V7t1-2 at different concentrations in Example 14;

FIG. 52A is a graph showing the results for the HeLa cell colony formation experiment in Example 14;

FIG. 52B is a graph showing the results for the MCF 10A cell colony formation experiment in Example 14;

FIG. 53 is a graph showing the results for the western blot assay for protein degradation in Example 15;

FIG. 54 is a graph showing the results for western blot assay for the protein degradation by AS1411-VH032 at different concentrations in Example 15;

FIG. 55 is a graph showing the results for western blot assay for the protein degradation by AS1411, VH032, AS1411-VH032, or AS1411+VH032 in Example 15;

FIG. 56 is a graph showing the results for western blot assay after using proteasome inhibitor MG132 in Example 15;

FIG. 57 is a graph showing the results for western blot assay after using si-NCL to silence NCL in Example 15;

FIG. 58 is a graph showing the results for the colony formation experiment in Example 15;

FIG. 59 is a graph showing the results for the cell apoptosis experiment in Example 15;

FIG. 60 shows a model-of-action diagram of the MDM2-targeting homo-PROTAC degrader in Example 16;

FIG. 61 is a graph showing the results for western blot assay for protein degradation in Example 16;

FIG. 62 is a graph showing the results for western blot assay for the protein degradation by AS1411−AS1411 at different concentrations in Example 16;

FIG. 63 is a graph showing the results for western blot assay for the protein degradation by AS1411, AS1411−AS1411, or AS1411+AS1411 in Example 16;

FIG. 64 is a graph showing the results for western blot assay after using proteasome inhibitor MG132 in Example 16;

FIG. 65 is a graph showing the results for western blot assay after using si-NCL to silence NCL in Example 16;

FIG. 66 is a graph showing the results for the colony formation experiment in Example 16;

FIG. 67 is a graph showing the results for the cell apoptosis experiment in Example 16.

### DETAILED DESCRIPTION

Unless otherwise defined, all scientific and technical terms used in the present disclosure have the same meaning as commonly understood by those skilled in the art to which the present disclosure relates.

The term “alkyl” refers to a hydrocarbon chain radical that is linear or branched and that does not contain unsaturated bonds, and that is linked to the rest of the molecule via a single bond. Typical alkyl groups contain 1 to 10 (e.g., 1, 2, 3, 4, 5, 6, 7, 8, 9, or 10) carbon atoms, preferably 1 to 6 carbon atoms; examples include methyl, ethyl, n-propyl, isopropyl, n-butyl, isobutyl, tert-butyl, n-pentyl, isopentyl, neopentyl, tert-pentyl, n-hexyl, isohexyl, etc. In the present disclosure, C₀ alkyl refers to H, i.e., C₀₋₁₀ alkyl (or C₀-C₁₀ alkyl) includes H and C₁₋₁₀ alkyl (or C₁-C₁₀ alkyl).

The term “alkylene” refers to hydrocarbyl (divalent alkyl) formed from an alkane molecule by losing two hydrogen atoms, which may be linear or branched and is linked to the rest of the molecule via a single bond. Typical alkylene groups herein contain 1 to 10 (e.g., 1, 2, 3, 4, 5, 6, 7, 8, 9, or 10) carbon atoms, preferably 1 to 6 carbon atoms; examples include methylene (—CH₂—), ethylene, propylene, butylene, etc. In the present disclosure, C₀ alkylene refers to a single bond, i.e., C₀₋₁₀ alkylene (or C₀-C₁₀ alkylene) includes a single bond and C₁₋₁₀ alkylene (or C₁-C₁₀ alkylene).

The term “cycloalkyl” refers to an alicyclic hydrocarbon, for example, containing 1 to 4 monocyclic and/or fused rings and β-18 carbon atoms, preferably β-10 (e.g., 3, 4, 5, 6, 7, 8, 9, or 10) carbon atoms, such as cyclopropyl, cyclobutyl, cyclopentyl, cyclohexyl, or adamantyl.

The term “alkoxy” refers to a substituent formed from a hydroxy group by substituting the hydrogen atom with alkyl, e.g., an alkoxy group containing 1-10 carbon atoms, such as methoxy, ethoxy, propoxy, or butoxy.

The term “alkylamino” refers to a substituent formed from an amino group (—NH₂) by substituting one or two of the hydrogen atoms with alkyl, e.g., an alkylamino group containing 1-10 carbon atoms, such as

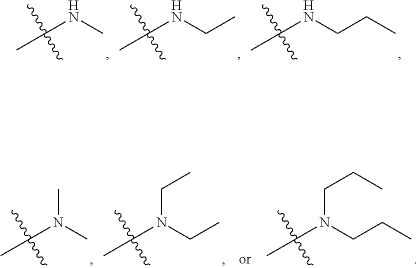

The term “halogen” refers to fluorine, chlorine, bromine, or iodine.

The term “haloalkyl” refers to a group formed from an alkyl group by substituting one or more hydrogen atoms with halogen (e.g., fluorine, chlorine, bromine, or iodine) atoms, e.g., —CHF₂, —CH₂F, —CF₃, —CH₂—CF₃, —CH₂CH₂—CF₃, or —CH₂CH₂CH₂—CF₃.

The term “aryl” refers to a monocyclic or polycyclic radical, including polycyclic radicals containing monoaryl and/or fused aryl groups, e.g., containing 1-3 monocyclic or fused rings and 6-18 (e.g., 6, 8, 10, 12, 14, 16, or 18) carbon ring atoms. The C₆-C₁₂ aryl described in the present disclosure refers to an aryl group containing 6-12 carbon ring atoms, such as phenyl, naphthyl, biphenyl, or indenyl.

The term “heterocyclyl” refers to a β- to 18-membered non-aromatic ring group containing 2 to 17 carbon atoms and 1 to 10 heteroatoms. The heterocyclyl may be a monocyclic, bicyclic, tricyclic, or tetracyclic ring system, which may include fused, spiro, or bridged ring systems. The heterocyclyl may be partially saturated (heteroaryl) or fully saturated (heterocycloalkyl). Suitable heteroaryl groups for the compound of the present disclosure contain 1, 2, or 3 heteroatoms selected from N, O, S, and P atoms and include, for example, coumarin, including 8-coumarin, quinolyl, including 8-quinolyl, isoquinolyl, pyridinyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl, and furopyridinyl. Suitable heterocycloalkyl groups for the compound of the present disclosure contain 1, 2, or 3 heteroatoms selected from N, O, and S atoms and include, for example, pyrrolidinyl, tetrahydrofuryl, dihydrofuran, tetrahydrothienyl, tetrahydrothiopyranyl, piperidinyl, morpholinyl, thiomorpholinyl, oxathianyl, piperazinyl, azetidinyl, oxetanyl, thietanyl, homopiperidinyl, oxiranyl, thiiranyl, azepinyl, oxazepanyl, diazepinyl, triazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H-indolyl, and quinolizinyl. In the present disclosure, for optionally substituted heterocyclyl, the substitution position may be any suitable carbon atom or heteroatom; for example, for

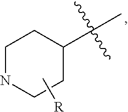

the substitution position for R may be on any suitable carbon atom or nitrogen atom, and it may be, for example,

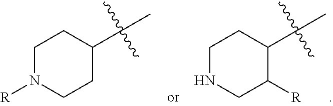

In the present disclosure, the term “oligonucleotide” consists of no more than 30 (e.g., 5, 10, 15, 20, 25, or 30) nucleotides (deoxyribonucleotides and/or ribonucleotides), and is single-stranded or double-stranded, particularly single-stranded. The oligonucleotide may comprise a chemical modification, a nucleic acid unit replacement, or a linkage to a functional group, wherein the chemical modification is that at least one base is modified, and the chemical modification includes at least one of phosphorylation, methylation, amination, sulfydrylation, isotopication, phosphorothioate backbone modification, methoxy modification, and fluoro modification; the nucleic acid unit replacement is that at least one nucleic acid unit is replaced by LNA, UNA, or GNA; the functional group includes at least one of a fluorophore, a radioactive group, biotin, digoxigenin, a nano-luminescent material, a nucleic acid substance, or an enzyme label.

In the present disclosure, for deoxyribonucleotides, A represents deoxyadenosine, T represents deoxythymidine, C represents deoxycytidine, and G represents deoxyguanosine; for ribonucleotides, A represents adenosine, G represents guanosine, C represents cytidine, and U represents uridine.

In the present disclosure, the term “oligopeptide” refers to a peptide consisting of 2-10 (e.g., 2, 3, 4, 5, 6, 8, or 10) amino acids, the “polypeptide” contains 11-50 (e.g., 15, 20, 30, 40, or 50) amino acids, and the “protein” contains no less than 50 amino acids.

The terms “patient” and “subject” and the like are used interchangeably herein and refer to any animal or cell thereof, whether in vitro or in situ, treated according to the method described herein. Specifically, the aforementioned animal includes mammals, for example, rats, mice, guinea pigs, rabbits, dogs, monkeys, or humans, particularly humans.

The term “treating” refers to preventing, curing, reversing, attenuating, alleviating, minimizing, suppressing, arresting, and/or stopping one or more clinical symptoms of a disease after its onset.

The term “preventing” refers to treatment to avoid, minimize, or make difficult the onset or progression of a disease prior to its onset.

The term “tumor” refers to an abnormal mass of tissue, wherein the growth of the mass exceeds and is uncoordinated with that of normal tissue. A tumor may be “benign” or “malignant”, depending on the following characteristics: degree of cell differentiation (including morphology and functionality), growth rate, local invasion, and metastasis. “Benign tumors” are generally well differentiated, characterized by growing slower than malignant tumors and remaining limited to the site of origin. In addition, benign tumors do not have the ability to infiltrate, invade, or metastasize to distant sites. In some cases, certain “benign” tumors may later result in malignant tumors, possibly due to additional genetic alterations in subpopulations of neoplastic cells of the tumors, and these tumors are referred to as “precancerous tumors”. “Malignant tumors” are usually poorly differentiated (anaplastic), characterized by rapid growth and accompanied by progressive infiltration, invasion, and destruction of surrounding tissues. In addition, malignant tumors generally have the ability to metastasize to distant sites.

The term “cancer” refers to malignant tumors (Stedman's Medical Dictionary, 25th ed.; Hensyl ed.; Williams & Wilkins: Philadelphia, 1990).

The term “autoimmune disease” refers to a disease caused by the body's immune response to self-antigens, resulting in damage to its own tissues.

The term “inflammation” refers to the body's defense response to stimuli, manifested as redness, swelling, heat, pain, dysfunction, and the like; it may be infectious inflammation caused by infection or non-infectious inflammation not caused by infection, such as inflammation caused by an immune response (e.g., various types of hypersensitivity and inflammation caused by some autoimmune diseases). The term “inflammatory disease” refers to a disease having inflammation.

The term “disease associated with pathogen infection” mainly refers to a disease caused by pathogen infection, and includes symptoms of body damage and responses to infection caused by pathogen invasion. A pathogen may be a microorganism (e.g., a virus, chlamydia, rickettsia, mycoplasma, a bacterium, a spirochete, a fungus, etc.), a parasite (a protozoan, a helminth, etc.), or other media. Particularly, the pathogen is a virus, for example, but not limited to, Adenoviridae (e.g., adenovirus), Herpesviridae (e.g., HSV1 (herpes of mouth), HSV2 (herpes of external genitalia), VZV (chicken pox), EBV (Epstein-Barr virus), CMV (cytomegalovirus)), Poxviridae (e.g., smallpox virus, vaccinia virus), Papovavirus (e.g., human papillomavirus (HPV)), Parvoviridae (e.g., B19 virus), Hepadnaviridae (e.g., hepatitis B virus), Polyomaviridae (e.g., polyomavirus), Reoviridae (e.g., reovirus, rotavirus), Picornaviridae (e.g., enterovirus, foot-and-mouth disease virus), Caliciviridae (e.g., Norwalk virus, hepatitis E virus), Togaviridae (e.g., rubella virus), Arenaviridae (e.g., lymphocytic choriomeningitis virus), Retroviridae (HIV-1, HIV-2, HTLV-1), Flaviviridae (e.g., Dengue virus, Zika virus, Japanese encephalitis virus, Chikungunya virus, yellow fever virus, hepatitis C virus, West Nile virus, etc.), Orthomyxoviridae (e.g., influenza viruses (e.g., influenza A virus, influenza B virus, influenza C virus, etc.)), Paramyxoviridae (e.g., human parainfluenza virus (HPIV) type 1, HPV type 2, HPV type 3, HPV type 4, Sendai virus, mumps virus, measles virus, respiratory syncytial virus, Newcastle disease virus, etc.), Bunyaviridae (e.g., California encephalitis virus, Hantavirus), Rhabdoviridae (e.g., rabies virus), Filoviridae (e.g., Ebola virus, Marburg virus), Coronaviridae (e.g., HCoV-229E, HCoV—OC43, HCoV-NL63, HCoV—HKU1, SARS-CoV, MERS-CoV, SARS-CoV-2, etc.), Astroviridae (e.g., astrovirus), and Bornaviridae (e.g., Borna virus).

The term “cardiovascular disease” refers to a class of diseases involving the heart or blood vessels.

The term “metabolic disease” refers to a disease caused by the accumulation or deficiency of certain metabolic substances such as sugars, fats, proteins, purines, calcium, and copper, when biochemical processes in vivo are disrupted.

The term “fibrosis” refers to a pathological process in which inflammation leads to necrosis of parenchymal cells in an organ, resulting in abnormal proliferation and excessive deposition of extracellular matrix within the tissue. In severe cases, fibrosis causes structural damage to tissues and results in organ sclerosis.

The disclosures of the various publications, patents, and published patent specifications cited herein are hereby incorporated by reference in their entireties.

To make the objectives, technical solutions, and advantages of the present disclosure clearer, the following further describes the present disclosure in detail with reference to the drawings and the examples. It should be understood that the specific examples described herein are merely intended to explain the present disclosure, and are not intended to limit the present disclosure. Unless otherwise specified, the experimental methods in the following examples are all conventional methods. Unless otherwise specified, the experimental materials used in the following examples were purchased from conventional biochemical reagent companies.

The sequences involved in the examples are shown in the table below:

TABLE 1
Sequence names and numbers

| Name | Sequence | No. |
|---|---|---|
| AS1411 | 5′-GGTGGTGGTGGTTGTGGTGGTGGTGG-3′ | SEQ ID NO: 1 |
| GRO29A | 5′-TTTGGTGGTGGTGGTTGTGGTGGTGGTGG-3′ | SEQ ID NO: 2 |
| GRO15A | 5′-GTTGTTTGGGGTGGT-3′ | SEQ ID NO: 3 |
| AT11 | 5′-TGGTGGTGGTTGTTGTGGTGGTGGTGGT-3′ | SEQ ID NO: 4 |
| S3-F | 5′-CTTCTGGGAAA-3′ | SEQ ID NO: 5 |
| S3-R | 5′-TTTCCCAGAAG-3′ | SEQ ID NO: 6 |
| MYC-F | 5′-GAGCACGTGGT-3′ | SEQ ID NO: 7 |
| MYC-R | 5′-ACCACGTGCTC-3′ | SEQ ID NO: 8 |
| R175Hapt | 5′-AUUAGCGCAUUUUAACAUAGGGUGC-3′ | SEQ ID NO: 9 |
| IncRV | 5′-CCUUUUGUUUUUCCCUCUCCAGG-3′ | SEQ ID NO: 10 |
| Inc V7 | 5′-UAUUUUUCCCUCUCCACCCU-3′ | SEQ ID NO: 11 |
| V7t1 | 5′-TGTGGGGGTGGACGGGCCGGGTAGA-3′ | SEQ ID NO: 12 |
| CRO | 5′-CCTCCTCCTCCTTCTCCTCCTCCTCC-3′ | SEQ ID NO: 13 |
| iSN04 | 5′-AGATTAGGGTGAGGGTGA-3′ | SEQ ID NO: 14 |
| NC | 5′-GGAATTCCCGGTGCGCCGATCGCCGGATATAACTT-3′ | SEQ ID NO: 15 |
| si-NC | 5′-UUCUCCGAACGUGUCACGUTT-3′ | SEQ ID NO: 16 |
| si-NCL | 5′-GGAUGACGACGACGACGAAGATT-3′ | SEQ ID NO: 17 |
| si-MDM2 | 5′-GCUUGGCCUACAGUCAUCUTT-3′ | SEQ ID NO: 18 |
| SL1 | 5′-ATCAGGCTGGATGGTAGCTCGGTCGGGGTGGGT | SEQ ID NO: 19 |
| | GGGTTGGCAAGTCTGAT-3′ | |

### Example 1

AS 1411 is a typical GRO having the sequence set forth in SEQ ID NO: 1. A GRO contains one or more GGT motifs and has stable G4 structural characteristics. A GRO is a guanine-rich oligonucleotide capable of specifically binding to NCL. In the present disclosure, the GRO is one of DNA aptamer AS 1411 and an AS 1411 derivative/analog, all of which have the stable G4 structural characteristics described above and can specifically bind to NCL.

(1) NCL can Bind to MJDM2, and AS 1411 does not Affect the Interaction Between NCL and MJDM2

Co-immunoprecipitation (Co-IP) is a classical method for the study of protein interactions based on the specific interaction between an antibody and an antigen. It is an effective method to determine the physiological interaction between two proteins in intact cells.

HeLa cervical cancer cells were lysed with an IP lysis solution (purchased from Thermo Scientific, Cat. No.: 87788) and then incubated with AS 1411 or iSN04 at different concentrations (0 μM, 1 μM, 5 μM, 10 μM, and 20 μM) at 4° C. for 6 h. Subsequently, 5 L of an antibody against NCL was added, and the mixture was further incubated at 4° C. overnight. Protein A/G magnetic beads (purchased from Thermo Scientific, Cat. No.: 88802) were added, and the mixture was incubated with rotation at room temperature for 1.5 h. The bound NCL and its interacting proteins on the magnetic beads were washed with an IP lysis buffer (purchased from Thermo Scientific, Cat. No.: 87788), and an SDS-PAGE protein loading buffer (purchased from Beyotime, Cat. No.: P0015) was added. The mixture was heated to 100° C. and held for 10 min. The magnetic beads were adsorbed on a magnetic rack, and the supernatant was transferred into a new tube. Subsequently, western blot assay was performed to detect whether NCL bound to MDM2 and whether the binding was affected by AS 1411 or iSN04. The experimental procedures of western blot assay were as follows: The protein sample was separated by SDS-PAGE electrophoresis, and the separated protein was transferred onto a PVDF membrane, which was blocked with a TBST buffer containing 5% skim milk at room temperature for 1 h; a primary antibody against MDM2 (purchased from proteintech, Cat. No.: 27883-1-AP) or a primary antibody against NCL (purchased from Cell Signaling Technology, Cat. No.: 14574S) was incubated overnight at 4° C.; after washing with TBST, the membrane was incubated with an HRP-labeled secondary antibody (purchased from ABclonal, Cat. No.: ASO14) at room temperature for 1 h; the protein band was visualized using an enhanced chemiluminescence detection kit (purchased from ABclonal, Cat. No.: RM00021P).

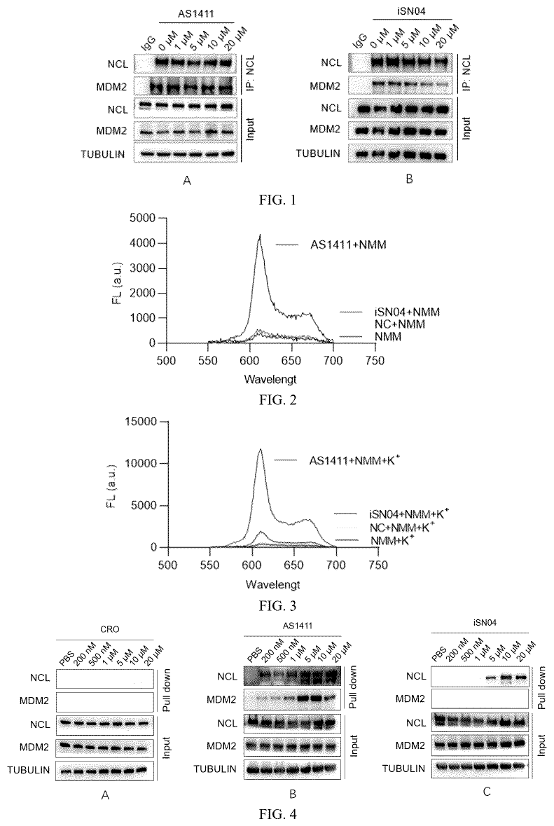

The results in FIG. 1A indicate that NCL can bind to MDM2 and that AS1411 does not affect the interaction between NCL and MDM2. The same experiment was performed on a derivative of AS1411, and the experimental results were similar to those of AS1411. However, the results in FIG. 1B indicate that NCL can bind to MDM2, but iSN04 blocks the binding of NCL to MDM2.

### (2) AS1411 is a GRO Having Stable G4 Structural Characteristics

Both AS1411 and iSN04 are oligonucleotides specifically binding to NCL. The sequence of AS1411 is SEQ ID NO: 1, and the sequence of iSN04 is SEQ ID NO: 14. AS1411 contains a plurality of GGT motifs, and iSN04 contains one GGT motif. Whether AS1411 and iSN04 have stable G4 structural characteristics was determined. The experimental process was as follows: 5 μM NC (negative control, SEQ ID NO: 15), AS1411, or iSN04 was added to water, and the total volume was 100 μL. The mixture was heated at 95° C. for 7 min and then immediately incubated on ice for 3 min, followed by the addition of 2 μL of N-methyl mesoporphyrin IX (NMM) (purchased from MCE, Cat. No.: HY-133821) to make a final concentration of 1 μM. The resulting mixture was incubated at room temperature for 1 h in the dark. The fluorescence intensity was detected by a microplate reader, with an emission wavelength of 550-700 nm and an excitation wavelength of 399. The results are shown in FIG. 2. No G4 structural characteristics were detected for iSN04, but AS1411 had stable G4 structural characteristics (fluorescence signal intensity of over 4000).

Guanine-rich DNA sequences can form more distinct G4 structural characteristics in the presence of metal ions (usually sodium and potassium ions). 5 μM NC (negative control, SEQ ID NO: 15), AS1411, or iSN04 was added to a 100 mM K⁺ solution, and the total volume was 100 μL. The mixture was well mixed, heated at 95° C. for 7 min, and then immediately incubated on ice for 3 min, followed by the addition of 2 μL of N-methyl mesoporphyrin IX (NMM) (purchased from MCE, Cat. No.: HY-133821) to make a final concentration of 1 μM. The resulting mixture was incubated at room temperature for 1 h in the dark. The fluorescence intensity was detected by a microplate reader, with an emission wavelength of 550-700 nm and an excitation wavelength of 399. The results are shown in FIG. 3. iSN04 had weak G4 structural characteristics (fluorescence signal intensity of 1000-2000), but AS1411 had more stable G4 structural characteristics (fluorescence signal intensity of over 10000).

The above results indicate that iSN04 can form a weak G4 structure only in the presence of K⁺ and cannot form G4 structural characteristics in the absence of K⁺; but AS1411 can form stable G4 structural characteristics in the presence or absence of K⁺.

### (3) AS1411 can Capture NCL and MDM2 in a Concentration-Dependent Manner

HeLa cervical cancer cells were lysed with an IP lysis solution (purchased from Thermo Scientific, Cat. No.: 87788) and then incubated with a CRO (cytosine-rich oligonucleotide, negative control, SEQ ID NO: 13), AS1411, or iSN04 labeled by biotin at the 5′ end at different concentrations (0 nM, 200 nM, 500 nM, 1 μM, 5 μM, 10 μM, and 20 μM) at 4° C. for 6h. Subsequently, streptavidin agarose gel beads (purchased from cytiva, Cat. No.: 17511301) were added, and the mixture was further incubated at 4° C. overnight. After the bound biotin-labeled CRO, AS411, or iSN04 and its captured proteins on the gel beads were washed multiple times with an IP lysis buffer (purchased from Thermo Scientific, Cat. No.: 87788), an SDS-PAGE protein loading buffer (purchased from Beyotime, Cat. No.: P0015) was added, and the mixture was heated to 100° C. and held for 10 min. After centrifugation, the supernatant was collected as a pull-down product. The pull-down product was detected by western blot assay.

The results in FIG. 4A indicate that the CRO cannot capture either NCL or MDM2; the results in FIG. 4B indicate that AS1411 can capture NCL and MDM2 in a concentration-dependent manner; the results in FIG. 4C indicate that iSN04 can capture NCL only at a high concentration, but iSN04 cannot capture MDM2.

(4) AS1411 Recruits MDM2 Depending on its Interaction with NCL

2 μg/mL recombinant human NCL (purchased from ACROBiosystems, NUL-H5253) and 2 μg/mL recombinant human MDM2 (purchased from R&D Systems, E3-202-050) were mixed well and incubated at 4° C. for 7 h.

Subsequently, 5 μL of an antibody against NCL was added, and the mixture was further incubated at 4° C. overnight. Protein A/G magnetic beads (purchased from Thermo Scientific, Cat. No.: 88802) were added, and the mixture was incubated with rotation at room temperature for 1.5 h. The magnetic beads were washed 3 times with a TBST buffer, and an SDS-PAGE protein loading buffer (purchased from Beyotime, Cat. No.: P0015) was added. The mixture was heated to 100° C. and held for 10 min. The magnetic beads were adsorbed on a magnetic rack, and the supernatant was transferred into a new tube. Subsequently, western blot assay was performed to detect whether NCL bound to MDM2. The results in FIG. 5A indicate that NCL can bind to MDM2.

6 μg/mL recombinant human NCL and 6 μg/mL recombinant human MDM2 were mixed well and incubated at 4° C. for 7 h. Subsequently, 400 nM CRO, AS1411, or iSN04 labeled by biotin at the 5′ end was added, and the mixture was incubated at 4° C. for 6 h. Streptavidin agarose gel beads (purchased from cytiva, Cat. No.: 17511301) were added, and the mixture was further incubated at 4° C. overnight. The gel beads were washed 4 times with a TBST buffer, and an SDS-PAGE protein loading buffer (purchased from Beyotime, Cat. No.: P0015) was added. The mixture was heated to 100° C. and held for 10 min. After centrifugation, the supernatant was collected. The pull-down product was then detected by western blot assay. The results in FIG. 5B indicate that the CRO cannot bind to either NCL or MDM2. The results in FIG. 5C indicate that AS1411 recruits MDM2 in large amounts depending on its interaction with NCL. The results in FIG. 5D indicate that iSN04 can only bind to NCL but cannot recruit MDM2 depending on its interaction with NCL.

### Example 2

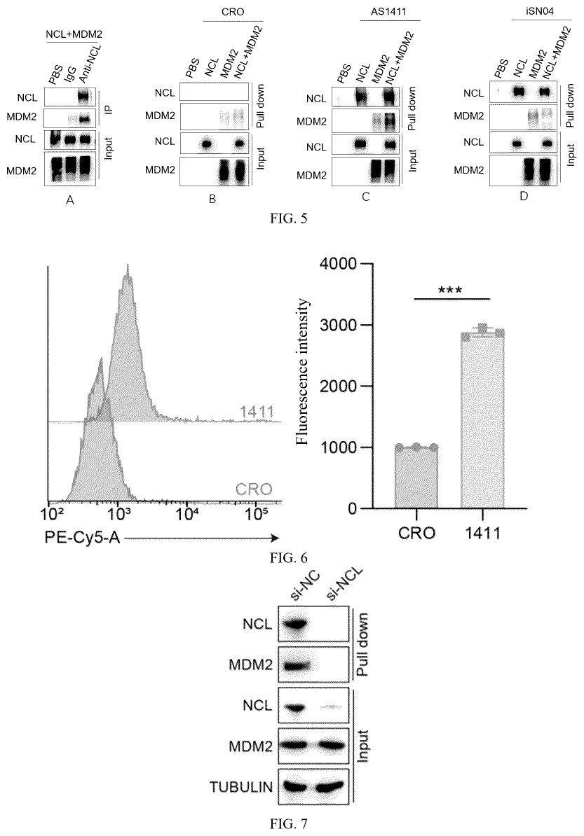

When AS1411 was selected as the GRO, the model analysis for AS1411 as the novel recruitment element of MDM2 and a PROTAC constructed by using AS1411 as the novel recruitment element of MDM2 was specifically as follows: Since NCL is highly expressed in tumor cells and specifically distributed on the surface of tumor cells, the binding ability of AS1411 to HeLa cervical cancer cells was examined. HeLa cells were incubated with 500 nM CRO (cytosine-rich oligonucleotide, negative control) and AS1411 labeled by Cy5 for 1.5 h. The binding ability to HeLa cells was detected by using a flow cytometer, and the results are shown in FIG. 6. The results indicate that AS1411 can significantly bind to HeLa cells. AS1411 is set forth in SEQ ID NO: 1, and the sequence of the CRO is set forth in SEQ ID NO: 13.

HeLa cells were transfected with negative control siRNA (si-NC, SEQ ID NO: 16) or NCL siRNA (si-NCL, SEQ ID NO: 17) and subsequently incubated with 500 nM biotin-labeled AS1411 for 6 h. After cell lysis, the supernatant was incubated with streptavidin-coated magnetic beads at 4° C. overnight. The bound AS1411 and its captured proteins on the magnetic beads were eluted with an eluent, and the pull-down products were detected by western blot assay.

The results are shown in FIG. 7. The results indicate that silencing NCL can block the recruitment of MDM2 by AS1411, indicating that AS1411 recruits MDM2 through its interaction with NCL.

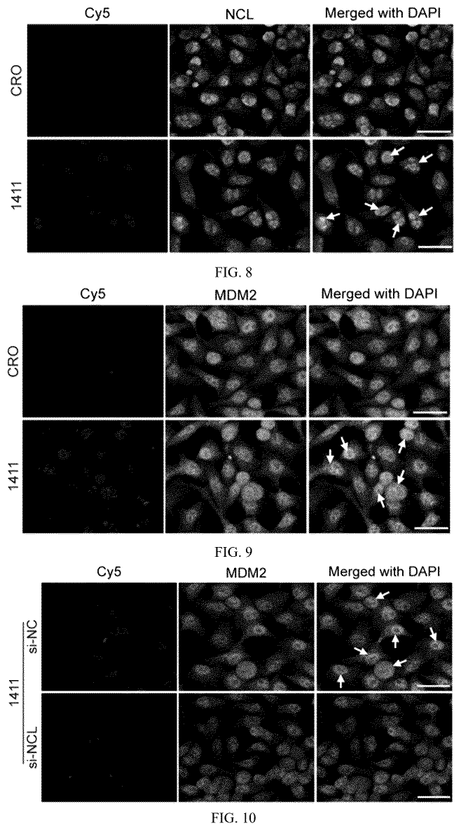

HeLa cells were incubated with 500 nM Cy5-labeled CRO or AS1411 for 6 h. After the incubation was completed, immunofluorescence staining was performed on NCL and MDM2, and 4′,6-diamidino-2-phenylindole (DAPI) was used to stain nuclei. The cell penetration of the CRO or AS1411 and the intracellular colocalization of the CRO or AS1411 with NCL and MDM2 were observed under a confocal laser microscope. The results are shown in FIGS. 8 and 9. The results indicate that AS1411 can penetrate HeLa cells and exhibits significant colocalization with both NCL and MDM2.

HeLa cells were transfected with si-NC or si-NCL and subsequently incubated with 500 nM Cy5-labeled AS1411 for 6 h. After the incubation was completed, immunofluorescence staining was performed on MDM2, and DAPI was used to stain nuclei. The intracellular colocalization of AS1411 with MDM2 was observed by a confocal laser microscope. The results are shown in FIG. 10. The results indicate that silencing NCL can block AS1411 from penetrating cells, with no observed colocalization between AS1411 and MDM2.

Brief PROTAC models based on the novel recruitment element GRO (e.g., AS1411) of MDM2 were constructed.

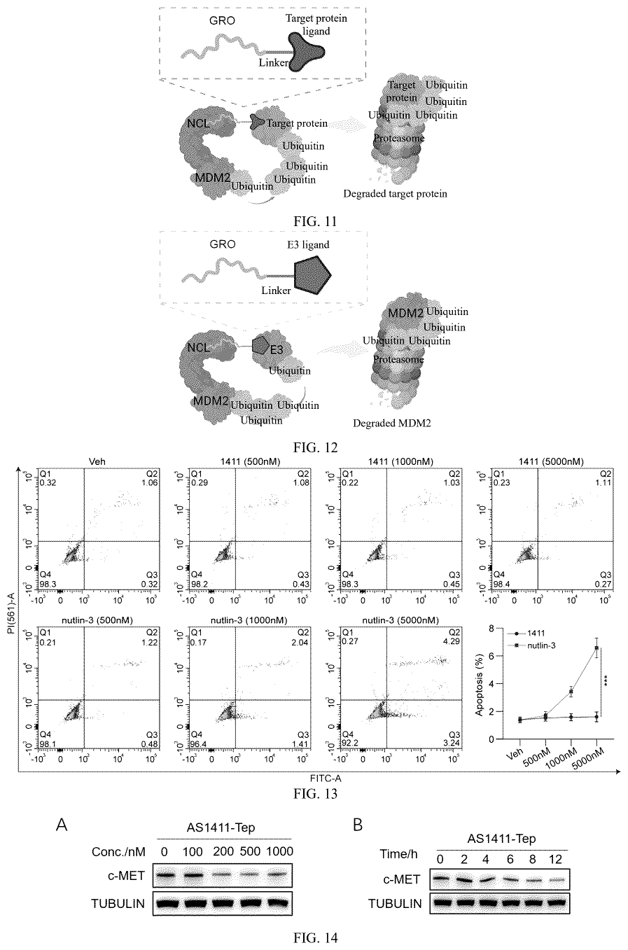

The models had two types. In one type, the novel recruitment element AS1411 of MDM2 was linked to a ligand of another target protein to form a PROTAC that leveraged the E3 ubiquitin ligase activity of MDM2 to ubiquitinate and degrade the another target protein. The possible mode of action is shown in FIG. 11. In the other type, MDM2 was taken as a target protein, and the novel recruitment element GRO (e.g., AS1411) of MDM2 was linked to a ligand of an E3 ubiquitin ligase to form a PROTAC that leveraged another E3 ubiquitin ligase to degrade MDM2. The possible mode of action is shown in FIG. 12.

That is, in the example shown in FIG. 11, MDM2 acts as an E3 ubiquitin ligase in the PROTAC, and the GRO (e.g., AS1411), as the novel recruitment element of MDM2, is conjugated to a target protein ligand via a linker to degrade the target protein. The target protein ligand includes, but is not limited to, small molecule compounds, oligonucleotides (including nucleic acid aptamers, etc.), oligopeptides, polypeptides, proteins (e.g., antibodies), and the like; the target protein is a protein that needs to be targeted for degradation, including but not limited to, mutant proteins, pseudokinases, transcription factors, scaffold proteins, membrane proteins, and the like.

In the example shown in FIG. 12, when MDM2 acts as a target protein in the PROTAC, the GRO (e.g., AS1411), as the novel recruitment element of MDM2, is conjugated to an E3 ubiquitin ligase ligand via a linker to degrade MDM2. The ligand of the E3 ubiquitin ligase includes, but is not limited to, small molecule compounds, oligonucleotides (including nucleic acid aptamers, etc.), oligopeptides, polypeptides, proteins (e.g., antibodies), and the like. The E3 ubiquitin ligase includes, but is not limited to, CRBN, VHL, IAP, cbl-b, MDM2, DCAF15, RNF114, DCAF16, KEAP1, FEM1B, and the like.

In the aforementioned preferred examples shown in FIGS. 11 and 12, the linker may be an oligonucleotide or any other small molecule or macromolecule with a linking function.

The toxicity of AS1411 to normal cells was compared with that of the small molecule ligand nutlin-3a of MDM2 to normal cells. AS1411 or nutlin-3a at different concentrations (0 nM, 500 nM, 1000 nM, and 5000 nM) was incubated with MCF 10A human normal mammary epithelial cells for 4 days, and the medium containing the respective concentrations of AS1411 or nutlin-3a was renewed every day. The cell apoptosis proportion was detected using a flow cytometer. The results are shown in FIG. 13. The results indicate that AS1411 has no significant toxicity, but nutlin-3a can cause cell apoptosis, indicating that AS1411 is superior to nutlin-3a in safety.

### Example 3: PROTAC Targeting c-MET

c-Mesenchymal-epithelial transition factor (c-MET) is one of receptor tyrosine kinases, and its ligand is hepatocyte growth factor (HGF). A typical hallmark of cellular carcinogenesis is the occurrence of epithelial-to-mesenchymal transition, in which c-MET is thought to play a key driving role. Upon binding to ligand HGF, c-MET undergoes dimerization and phosphorylation at multiple juxtamembrane domain sites, thereby becoming activated. This activates a series of downstream signaling pathways, primarily including PI3K/Akt, MAPK, FAK, RAS, STAT, and the like. The activation of these pathways initiates more complex and extensive regulatory networks. Under normal physiological conditions, HGF/c-MET can mediate embryonic development, cell proliferation, damaged tissue repair, and neuromuscular formation. Numerous studies have shown that the HGF/c-MET signaling pathway is abnormally activated in tumor cells, promoting the growth, invasion, migration, and angiogenesis of the tumor cells. Abnormal activation of the HGF/c-MET signaling pathway can be achieved through a variety of mechanisms, including: 1. overexpression of the c-MET protein; 2. gene mutation of c-MET; 3. overexpression of HGF. The HGF/c-MET signaling pathway exhibits abnormal expression or mutation in various types of solid tumors, such as lung cancer, gastric cancer, liver cancer, breast cancer, skin cancer, and large intestine cancer, and plays an important role in the development and progression of various tumors.

In this example, a PROTAC capable of degrading c-MET was prepared. The PROTAC capable of degrading c-MET was formed by conjugating a novel recruitment element GRO of ubiquitin ligase MDM2 and a ligand of c-MET via a linker. The GRO includes DNA aptamer AS1411 and a derivative or analog thereof (e.g., GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M). The PROTAC can effectively degrade c-MET. In this example, DNA aptamer AS1411 was selected as the GRO.

The ligand of c-MET includes small molecule compounds, oligonucleotides (e.g., nucleic acid aptamers, etc.), oligopeptides, polypeptides, proteins (e.g., antibodies), and the like. In a preferred example of the present disclosure, the ligand of c-MET includes tepotinib.

Tepotinib was used as a c-MET ligand and conjugated to AS1411 through a chemical linker to prepare the PROTAC.

The specific procedures were as follows:

### (1) Synthesis of Compound 3

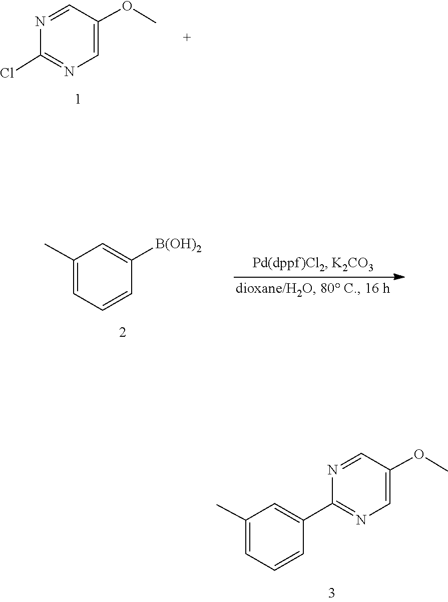

Compound 2 (23.5 g, 172.94 mmol), K₂CO₃ (47 g, 345.88 mmol), and Pd(dppf)Cl₂ (6.2 g, 8.65 mmol) were added to a solution of compound 1 (25 g, 172.94 mmol) in dioxane (450 mL) and H₂O (150 mL). The mixture was stirred at 80° C. for 16 h under a nitrogen atmosphere. LCMS showed that the reaction was completed. Dilution was performed with water (300 mL), and extraction was performed with EtOAc (3×100 mL). The organic layers were combined and washed with a saturated aqueous NaCl solution (300 mL). The organic phase was dried over anhydrous Na₂SO₄, filtered, and concentrated under vacuum. The residue was purified by flash chromatography and eluted with EA/PE (0-30%) to give compound 3 (32 g, yield: 94%) as a brown solid.

LCMS: [M+H]⁺=201.2, Rt=2.13 min, purity: 96.34% (214 nm).

¹HNMR (400 MHz, DMSO-d₆) δ 8.63 (s, 2H), 8.20-8.05 (m, 2H), 7.40-7.36 (m, 1H), 7.29-7.27 (m, 1H), 3.96 (s, 3H), 2.40 (s, 3H).

### (2) Synthesis of Compound 4

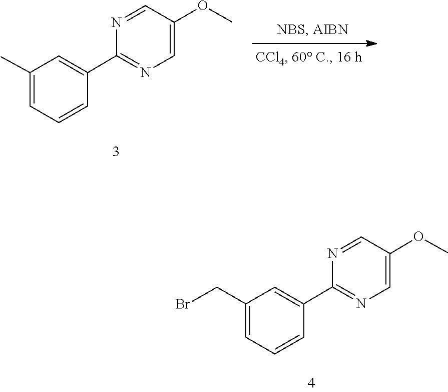

AIBN (1.2 g, 7.5 mmol) was added to a solution of compound 3 (15 g, 75 mmol) and NBS (13 g, 75 mmol) in CCl4 (200 mL). The mixture was stirred at 60° C. for 16 h under a nitrogen atmosphere. LCMS showed that the reaction was completed. The solvent was removed by concentration under reduced pressure. Dilution was performed with water (300 mL), and extraction was performed with EtOAc (3×150 mL). The organic layers were combined and washed with a saturated aqueous NaCl solution (300 mL). The organic substance was dried over anhydrous Na₂SO₄, filtered, and concentrated under vacuum to give compound 4 (crude, 21 g) as a brown solid.

LCMS: [M+H]⁺=279.0, Rt=0.99 min, purity: 57% (214 nm).

### (3) Synthesis of Compound 6

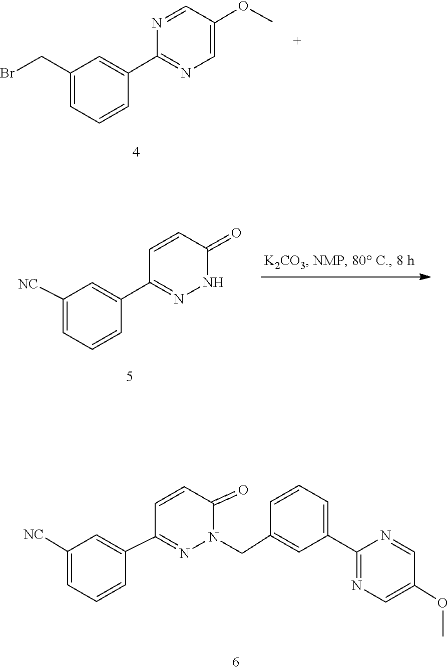 [SMILES: COc1cnc(-c2cccc(CBr)c2)nc1 ; N#Cc1cccc(-c2ccc(=O)[nH]n2)c1 ; COc1cnc(-c2cccc(Cn3nc(-c4cccc(C#N)c4)ccc3=O)c2)nc1]

Compound 5 (5.6 g, 28.78 mmol) was added to a stirred solution of compound 4 (8 g, 28.78 mmol) and K₂CO₃ (4.3 g, 31.65 mmol) in NMP (100 mL). The mixture was stirred at 80° C. for 16 h. LCMS showed that the reaction was completed. After cooling to room temperature, water (200 mL) was added to the reaction solution. A large amount of solid was precipitated and filtered under vacuum, and the filter cake was washed with EA (200 mL), filtered under vacuum, and dried under vacuum to give compound 6 (5.8 g, crude) as an off-white solid.

LCMS: [M+H]⁺=396.0, Rt=1.17 min, purity: 90% (214 nm)

¹HNMR (400 MHz, DMSO-d₆) δ 8.65-8.64 (m, 2H), 8.43-8.34 (m, 2H), 8.27-8.20 (m, 2H), 8.19-8.16 (m, 1H), 7.94-7.92 (m, 1H), 7.74-7.70 (m, 1H), 7.49-7.48 (m, 2H), 7.18-7.15 (m, 1H), 5.45 (s, 2H), 3.95 (s, 3H)

### (4) Synthesis of Compound 7

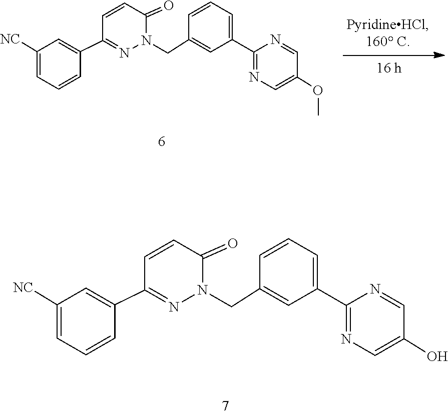 [SMILES: COc1cnc(-c2cccc(Cn3nc(-c4cccc(C#N)c4)ccc3=O)c2)nc1 ; N#Cc1cccc(-c2ccc(=O)n(Cc3cccc(-c4ncc(O)cn4)c3)n2)c1]

Compound 6 (5.5 g, 14.68 mmol) and pyridine-HCl (34 g) were added to a flask (500 mL). The mixture was stirred at 160° C. for 16 h. LCMS showed that the reaction was completed. After cooling to room temperature, water (300 mL) was added to the reaction solution. A large amount of solid was precipitated and filtered under vacuum, and the filter cake was washed with EA (200 mL), filtered under vacuum, and dried under vacuum to give compound 7 (4.7 g, crude) as an off-white solid.

LCMS: [M+H]⁺=382.2, Rt=1.25 min, purity: 90% (214 nm)

### (5) Synthesis of Compound 9

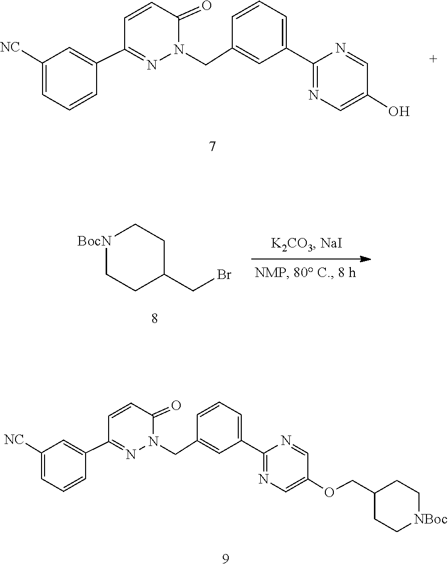 [SMILES: N#Cc1cccc(-c2ccc(=O)n(Cc3cccc(-c4ncc(O)cn4)c3)n2)c1 ; CC(C)(C)OC(=O)N1CCC(CBr)CC1 ; CC(C)(C)OC(=O)N1CCC(COc2cnc(-c3cccc(Cn4nc(-c5cccc(C#N)c5)ccc4=O)c3)nc2)CC1]

NaI (0.18 g, 1.23 mmol) and K₂CO₃ (3.4 g, 24.67 mmol) were added to a suspension of compound 7 (4.7 g, 12.33 mmol) in NMP (500 mL), followed by the addition of compound 8 (4.1 g, 14.80 mmol). The resulting solution was stirred at 80° C. for 8 h. After cooling to room temperature, water (300 mL) was added to the reaction solution. A large amount of solid was precipitated and filtered under vacuum, and the filter cake was washed with EA (300 mL), filtered under vacuum, and dried under vacuum to give compound 9 (4 g, crude) as an off-white solid.

LCMS: [M−55]⁺=523.2, Rt=1.53 min, purity: 71% (214 nm)

### (6) Synthesis of Compound 10

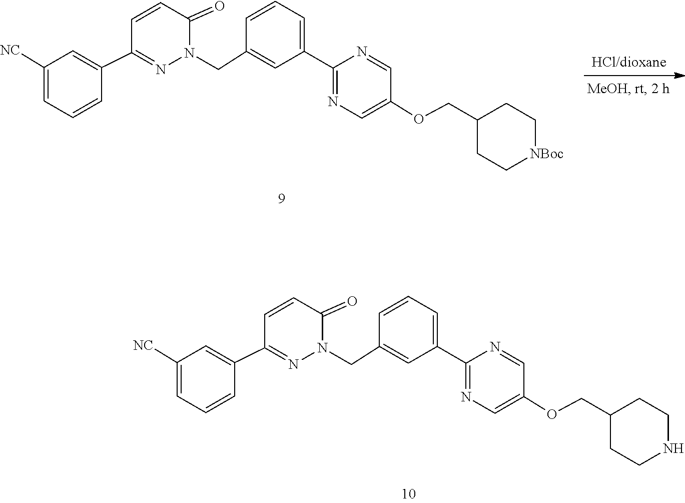 [SMILES: CC(C)(C)OC(=O)N1CCC(COc2cnc(-c3cccc(Cn4nc(-c5cccc(C#N)c5)ccc4=O)c3)nc2)CC1 ; N#Cc1cccc(-c2ccc(=O)n(Cc3cccc(-c4ncc(OCC5CCNCC5)cn4)c3)n2)c1]

MeOH (2 mL) was added to a solution of compound 9 (1 g, 1.73 mmol) in HCl/dioxane (4 M, 6 mL). The resulting solution was stirred at room temperature for 2 h. The mixture was concentrated under vacuum to give a crude product (1.2 g, crude).

LCMS: [M+H]⁺=479.2, Rt=1.67 min, purity: 67% (214 nm)

### (7) Synthesis of Compound 12

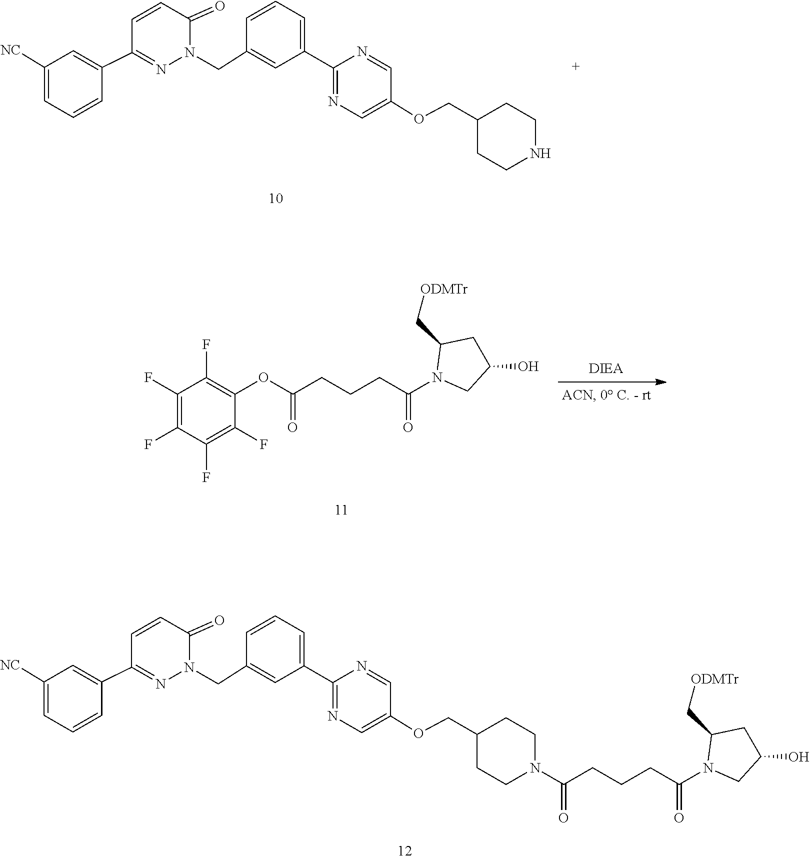

DIEA (1.6 g, 12.55 mmol) was added to a solution of compound 10 (1.0 g, 2.09 mmol) in ACN (20 mL) under a nitrogen atmosphere, and the mixture was stirred for 5 min in an ice bath. Subsequently, compound 11 (1.46 g, 2.09 mmol, in 10 mL of ACN) was added dropwise to the mixture, and the resulting mixture was stirred at room temperature for about 10 h and concentrated under vacuum to give a crude product. The product was purified by SGC (MeOH:DCM, 0% to 10%) to give compound 12 (1.0 g, crude) as a white solid.

LCMS: [M+Na]⁺=1016.7, Rt=6.61 min, purity: 93% (214 nm)

### (8) Synthesis of Compound 13

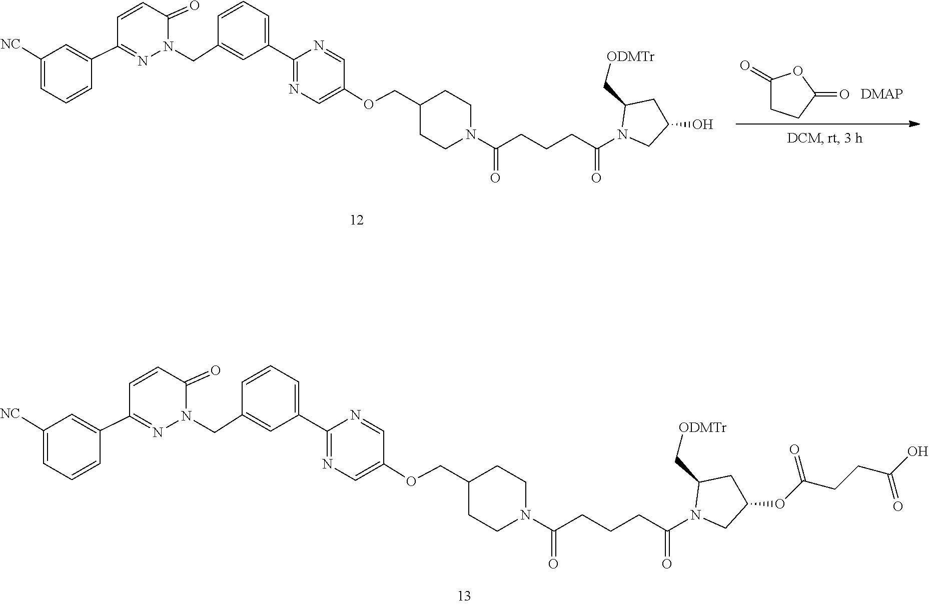

Succinic anhydride (1.5 mg, 1.5 mmol) and DMAP (0.37 g, 3.0 mmol) were added to a solution of compound 12 (1 g, 1 mmol) in DCM (20 mL) under a nitrogen atmosphere. The mixture was then stirred at room temperature for about 3 h. The solvent was removed by concentration under reduced pressure, and the residue was purified by reversed-phase chromatography (ACN (0.02% TEA)/water=40%-45%) to give compound 13 (840 mg, yield: 77%) as a white solid.

LCMS: [M+Na]⁺=1116.7, Rt=7.79 min, purity: 100% (214 nm)

¹HNMR (400 MHz, DMSO-d₆) δ 8.64-8.63 (m, 2H), 8.38-8.37 m, 2H), 8.26-8.23 (m, 2H), 8.18-8.16 (m, 1H), 7.94-7.92 (m, 1H), 7.73-7.71 (m, 1H), 7.49-7.48 (m, 2H), 7.31-7.29 (m, 4H), 7.22-7.15 (m, 6H), 6.88-6.86 (m, 4H), 5.44 (s, 2H), 5.31-5.24 (m, 1H), 4.42-4.40 (m, 1H), 4.21 (s, 1H), 4.05-4.03 (m, 2H), 3.91-3.75 (m, 2H), 3.73 (s, 6H), 3.56-3.33 (m, 1H), 3.45 (s, 3H), 3.28-3.21 (m, 3H), 3.09 (s, 1H), 2.98-2.94 (m, 2H), 2.56-2.51 (m, 1H), 2.36-2.27 (m, 3H), 2.34-2.19 (m, 2H), 2.04-1.98 (m, 2H), 1.79-1.67 (m, 3H), 1.23-1.04 (m, 2H).

### (9) Synthesis of Compound 13-CPG

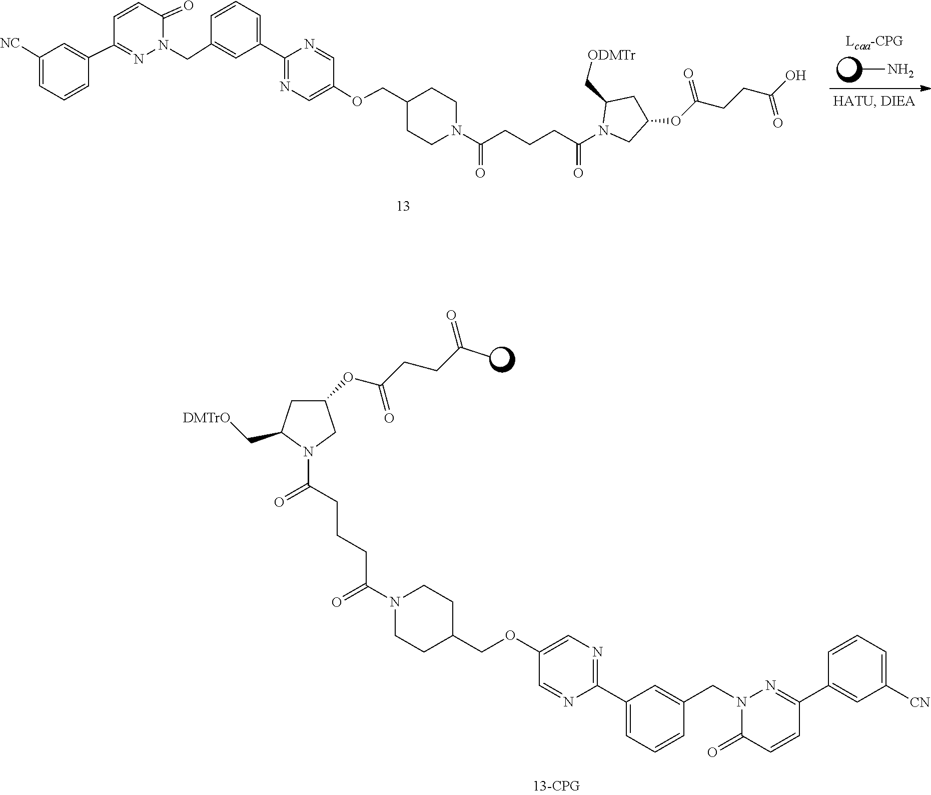

HATU (160 mg, 0.416 mmol), DIEA (160 μL), and lcaa-CPG (1000° A, 2000 mg) were added to a solution of compound 13 (400 mg, 0.366 mmol) in ACN (24.0 mL) at room temperature, and the mixture was shaken for 12 h. After the reaction was completed, CPG was washed with ACN, and CAP A (acetic anhydride:tetrahydrofuran=1:9, v/v, 8.0 mL) and CAP B (N-methylimidazole:pyridine:acetonitrile=15:10:75, v/v/v, 8.0 mL) were added. The mixture was shaken at room temperature for 1 h. The mixture was then filtered and washed 3 times with ACN (2 mL). After the mixture was lyophilized, 13-CPG (2000 mg) was obtained as a white powder.

### (10) Synthesis of AS1411-Tep

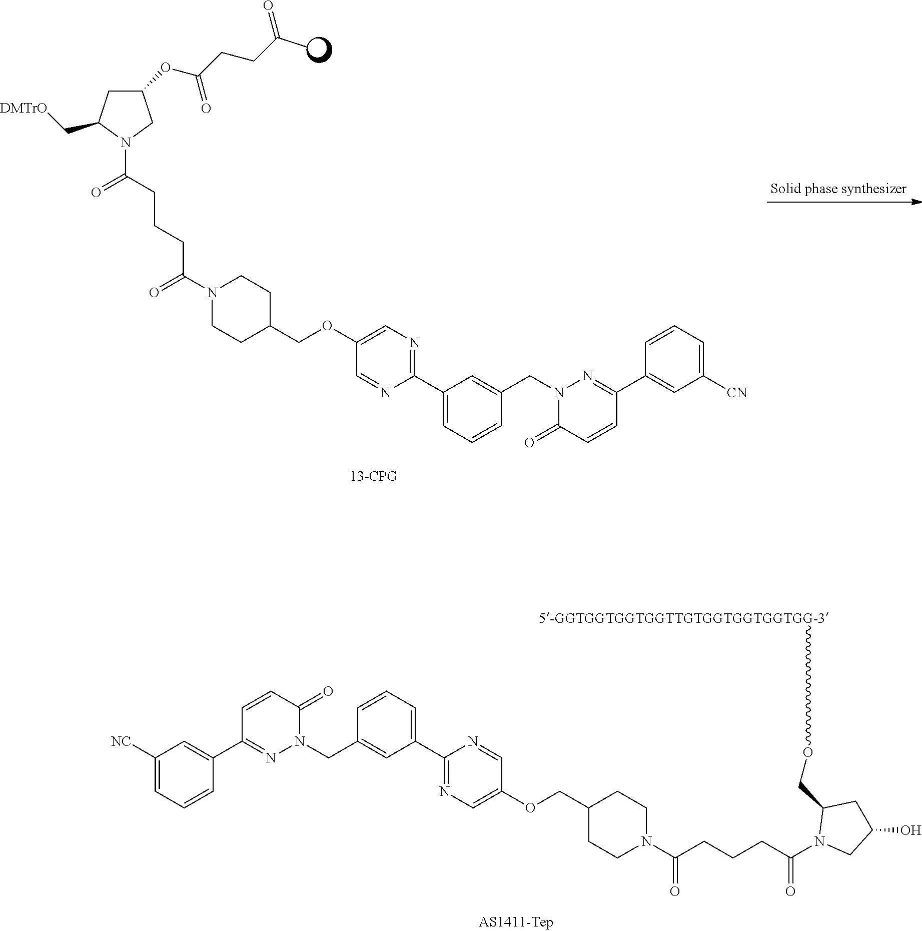

13-CPG (60 mg×16) was loaded into synthesis columns and processed on a K-A H-8 solid-phase synthesizer for synthesis. The solid-phase synthesis comprises four steps: detritylation, conjugation, capping, and oxidation. After the reaction was completed, 1.5 mL of ammonia solution:TEA (10:1) was added to CPG in each synthesis column, and the mixture was heated in an oven at 55° C. for 3 h. The supernatant was then collected and washed with water (1 mL×3). The crude product was purified by a protein purification system (Sepure, SDA) (column: NanoQ 15L, 4.7 mL; method: mobile phase A: a 20 mM aqueous NaOH solution, mobile phase B: an aqueous solution of 20 mM NaOH+2.0 M NaCl) to give AS1411-Tep (32.4 mg, purity=96.59%) as a white powder.

UPLC-MS (WATERS ACQUITY PREMIER): AS1411-Tep-UPLC, m/z=9025.95414 [M]⁻ (deconvolution); Rt=11.672 min (260 nm). Mass error<50 ppm.

HPLC: AS1411-Tep-HPLC, Rt: 10.411 min (260 nm), purity: 96.593%.

By referring to the aforementioned steps, iSN04-Tep (31.9 mg, purity=95.04%) was also prepared as a white powder.

UPLC-MS (WATERS ACQUITY PREMIER): iSN04-Tep-UPLC, m/z=6437.3592 [M]⁻ (deconvolution); Rt=10.354 min (260 nm). Mass error<50 ppm.

HPLC: iSN04-Tep-HPLC, Rt=11.789 min (260 nm), purity: 95.042%.

The following was performed to further verify the ability of the PROTAC products prepared based on AS1411 and iSN04 to degrade c-MET at the protein level, as detailed below.

(1) HOS osteosarcoma cells were incubated with AS1411-Tep at concentrations of 0 nM, 100 nM, 200 nM, 500 nM, and 1000 nM. After 8 h, cell samples were collected. The total protein was extracted using an RIPA lysis buffer (purchased from Beyotime, Cat. No.: P0013B), and the degradation of the c-MET protein was detected by western blot assay. The process was as follows: The protein sample was separated by SDS-PAGE electrophoresis, and the separated protein was transferred onto a PVDF membrane, which was blocked with a TBST buffer containing 5% skim milk at room temperature for 1 h; a primary antibody against c-MET was incubated overnight at 4° C.; after washing with TBST, the membrane was incubated with an HRP-labeled secondary antibody at room temperature for 1 h; the protein band was visualized using an enhanced chemiluminescence detection kit (purchased from ABclonal, Cat. No.: RM00021P).

The results are shown in FIG. 14A. AS1411-Tep can reduce the protein level of c-MET in a concentration-dependent manner.

(2) HOS osteosarcoma cells were incubated with 200 nM AS1411-Tep, and corresponding cell samples were collected at time gradients of 0 h, 2 h, 4 h, 6 h, 8 h, and 12 h for protein extraction and subsequent western blot assay to detect the degradation of the c-MET protein.

The results are shown in FIG. 14B. AS1411-Tep can reduce the protein level of c-MET in a time-dependent manner.

(3) HOS osteosarcoma cells were incubated with iSN04-Tep at concentrations of 0 nM, 50 nM, 100 nM, 200 nM, 500 nM, and 1000 nM. After 8 h, cell samples were collected. The total protein was extracted using an RIPA lysis buffer (purchased from Beyotime, Cat. No.: P0013B), and the degradation of the c-MET protein was detected by western blot assay. The process was as follows: The protein sample was separated by SDS-PAGE electrophoresis, and the separated protein was transferred onto a PVDF membrane, which was blocked with a TBST buffer containing 5% skim milk at room temperature for 1 h; a primary antibody against c-MET was incubated overnight at 4° C.; after washing with TBST, the membrane was incubated with an HRP-labeled secondary antibody at room temperature for 1 h; the protein band was visualized using an enhanced chemiluminescence detection kit (purchased from ABclonal, Cat. No.: RM00021P).

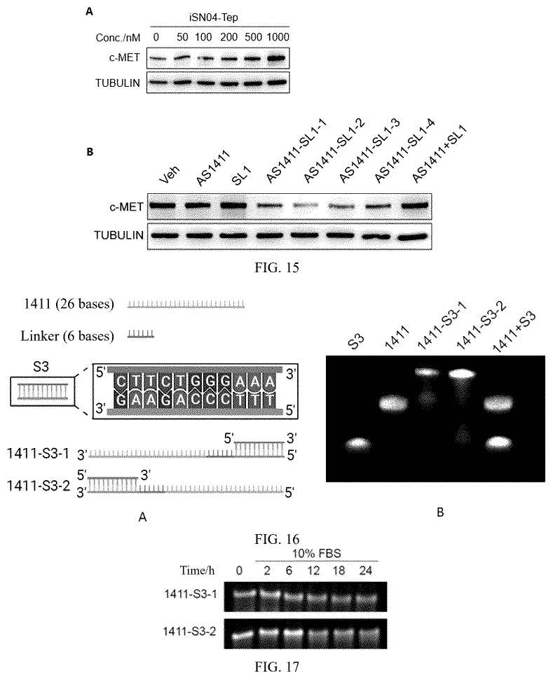

The results are shown in FIG. 15A. iSN04-Tep cannot reduce the protein level of c-MET in a concentration-dependent manner.

In this example, another PROTAC capable of degrading c-MET was prepared. The PROTAC capable of degrading c-MET was formed by conjugating a novel recruitment element GRO of ubiquitin ligase MDM2 and a ligand of c-MET via a linker. The GRO includes DNA aptamer AS1411 and a derivative or analog thereof (e.g., GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M). The PROTAC can effectively degrade c-MET. In this example, DNA aptamer AS1411 was selected as the GRO.

The ligand of c-MET includes small molecule compounds, oligonucleotides (e.g., nucleic acid aptamers, etc.), oligopeptides, polypeptides, proteins (e.g., antibodies), and the like. In a preferred example of the present disclosure, the ligand of c-MET includes DNA aptamer SL1 having the sequence set forth in SEQ ID NO: 19. The c-MET ligand was conjugated to AS1411 via a linker to prepare the PROTAC capable of degrading c-MET.

The linker of the PROTAC includes a double-stranded DNA oligonucleotide with β-30 A-T pairs, and more preferably, the linker of the PROTAC includes a double-stranded DNA oligonucleotide with 6 or 10 A-T pairs.

The preparation method for the PROTAC capable of degrading c-MET was as follows: The 3′ end of AS1411 was linked to the single strand with 6 T bases of the double-stranded DNA oligonucleotide by solid-phase synthesis to form a first conjugate product;
the 5′ end or 3′ end of SL1 was linked to the single strand with 6 A bases of the double-stranded DNA oligonucleotide by solid-phase synthesis to form a second conjugate product;
the first conjugate product and the second conjugate product were separately heated in a metal bath at 95° C. for 5 min, cooled to 37° C., left to stand for 30 min, and mixed in a molar ratio of 1:1, so that the novel PROTAC for c-MET, i.e., AS1411-SL1-1 or AS1411-SL1-2, was constructed by base complementary pairing of the double-stranded DNA oligonucleotide;
or
the 3′ end of AS1411 was linked to the single strand with 10 T bases of the double-stranded DNA oligonucleotide by solid-phase synthesis to form a third conjugate product;
the 5′ end or 3′ end of SL was linked to the single strand with 10 Abases of the double-stranded DNA oligonucleotide by solid-phase synthesis to form a fourth conjugate product;
the third conjugate product and the fourth conjugate product were separately heated in a metal bath at 95° C. for 5 min, cooled to 37° C., left to stand for 30 min, and mixed in a molar ratio of 1:1, so that the novel PROTAC for c-MET, i.e., AS1411-SL1-3 or AS1411-SL1-4, was constructed by base complementary pairing of the double-stranded DNA oligonucleotide.

In a further preferred example of the present disclosure, a derivative or analog of AS1411 could also be used to conjugate to SL1 via a DNA/double-stranded oligonucleotide to form a PROTAC product, and the procedures are the same as those with AS1411 being used and will not be repeated herein.

The following was performed to further verify the ability of the novel PROTAC product prepared based on AS1411 for c-MET, i.e., AS1411-SL1-1, AS1411-SL1-2, AS1411-SL1-3, or AS1411-SL1-4, to degrade c-MET at the protein level, as detailed below.

HOS cells were incubated with 500 nM AS1411-SL1-1, AS1411-SL1-2, AS1411-SL1-3, or AS1411-SL1-4. After 6 h, corresponding cell samples were collected for protein extraction and subsequent western blot assay to detect protein degradation. The results are shown in FIG. 15B. The results indicate that AS1411-SL1-1, AS1411-SL1-2, AS1411-SL1-3, and AS1411-SL1-4 can significantly reduce the protein level of c-MET.

### Example 4: PROTAC Targeting STAT3

In this preferred example, the use of AS1411 of the present disclosure as a novel recruitment element of MDM2 in the construction of a PROTAC is illustrated by an example of a transcription factor protein as an undruggable or difficult-to-drug protein target. Transcription factor proteins bind to specific sites on a DNA sequence to initiate gene transcription. It is difficult to prevent functional interactions between nuclear transcription factor proteins and DNA or co-regulatory proteins by using small molecules. This is because the sites on transcription factors involved in these interactions are usually large and flat surface regions, which are in contrast to the deep pharmaceutically acceptable binding pockets found on most enzymes or receptors, and hence transcription factor proteins are defined as undruggable or difficult-to-drug protein targets.

STAT3's over-activation in various tumors and its role as a cancer driver and a modulator of the tumor microenvironment have been verified in multiple studies, and STAT3 has become an attractive target in oncology and immuno-oncology. In this preferred example, the aforementioned beneficial effects of the use of a GRO (e.g., AS1411) as a novel recruitment element of MDM2 in the construction of a PROTAC can be verified by preparing a PROTAC capable of degrading transcription factor STAT3.

In this example, a PROTAC capable of degrading transcription factor STAT3 was prepared. Double-stranded transcription factor decoy oligonucleotide S3 that specifically recognizes STAT3 was taken as a target protein ligand, and the target protein ligand was conjugated to AS1411 via a single-stranded DNA linker to prepare the PROTAC capable of degrading transcription factor STAT3. The specific procedures were as follows.

Transcription factor decoy oligonucleotides can bind to transcription factors and belong to macromolecular ligands, and essentially, they are double-stranded DNA sequences artificially synthesized to mimic the binding sites of transcription factors in the promoter regions of target genes. Double-stranded transcription factor decoy oligonucleotide S3 that specifically recognizes STAT3 was taken as a target protein ligand and conjugated to AS1411 via a single-stranded DNA linker with 6 A bases.

Preferably, the conjugation procedures comprise: linking the antisense strand S3-R (SEQ ID NO: 6) of double-stranded transcription factor decoy oligonucleotide S3, a single-stranded DNA linker (5′-AAAAAA-3′), and AS1411 together by solid-phase synthesis, mixing with the sense strand S3-F (SEQ ID NO: 5) of double-stranded transcription factor decoy oligonucleotide S3 according to a molar ratio of 1:1, heating in a metal bath at 95° C. for 5 min, slowly annealing to room temperature, and completing the construction of the PROTAC by subjecting the nucleic acid sequences to base complementary pairing. The conjugation occurred at the 5′ end or 3′ end of AS1411, forming two PROTAC products, i.e., a first PROTAC product 1411-S3-1 and a second PROTAC product 1411-S3-2, as shown in FIG. 16A. The sequences used in the aforementioned reactions can be synthesized by Sangon Biotech (Shanghai) Co., Ltd.

The purity and size of the first PROTAC product 1411-S3-1 and the second PROTAC product 1411-S3-2 were detected using 20% native PAGE gel electrophoresis. Double-stranded transcription factor decoy oligonucleotide S3, AS1411, and a physical mixture (AS1411+S3) of AS1411 and double-stranded transcription factor decoy oligonucleotide S3 served as controls, and the results are shown in FIG. 16B. The results indicate that: the first PROTAC product 1411-S3-1 and the second PROTAC product 1411-S3-2 exhibit relatively high purity, a molecular weight higher than that of double-stranded transcription factor decoy oligonucleotides S3 and AS1411, and relatively good linking efficiency.

The constructed first PROTAC product 1411-S3-1 and second PROTAC product 1411-S3-2 were incubated in a DMEM complete medium containing 10% FBS for 0 h, 2 h, 4 h, 6 h, 8 h, 10 h, and 12 h, and the samples were then collected and tested for serum stability. The results are shown in FIG. 17. The results indicate that: the first PROTAC product 1411-S3-1 and the second PROTAC product 1411-S3-2 have relatively good serum stability.

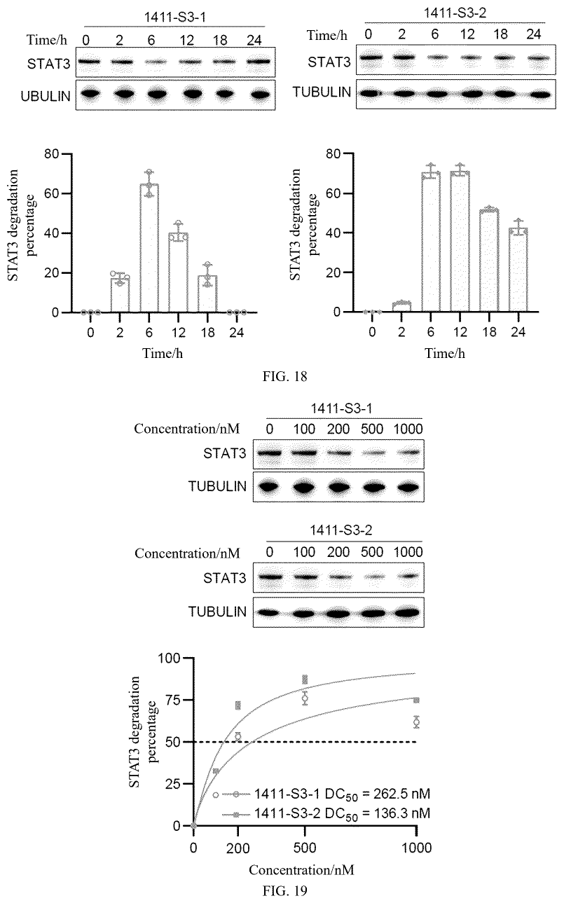

HeLa cells were incubated with 500 nM said first PROTAC product 1411-S3-1 or second PROTAC product 1411-S3-2, and corresponding cell samples were collected at time gradients of 0 h, 2 h, 6 h, 12 h, 18 h, and 24 h for protein extraction and subsequent western blot assay. The results are shown in FIG. 18. The results indicate that both the first PROTAC product 1411-S3-1 and the second PROTAC product 1411-S3-2 can reduce the protein level of transcription factor STAT3 in a time-dependent manner, and the effect of the first PROTAC product 1411-S3-1 is better than that of the second PROTAC product 1411-S3-2.

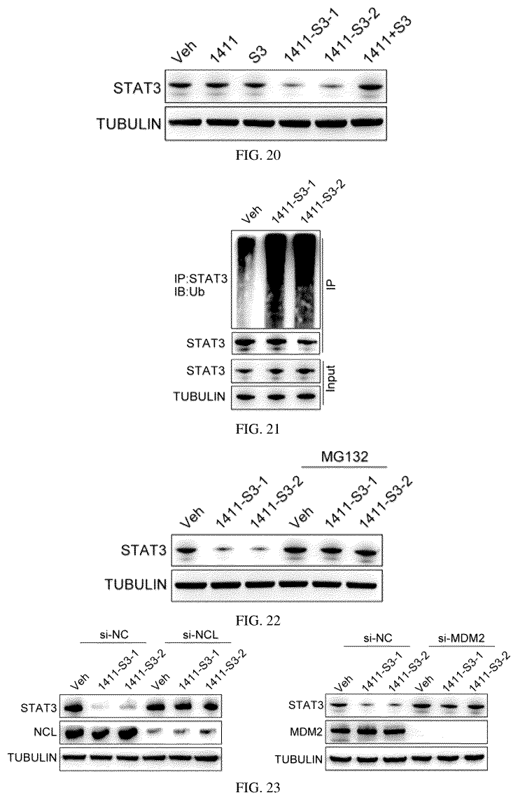

HeLa cells were incubated with the first PROTAC product 1411-S3-1 or the second PROTAC product 1411-S3-2 at concentrations of 0 nM, 100 nM, 200 nM, 500 nM, and 1000 nM. After 6 h, cell samples were collected for protein extraction and subsequent western blot assay. The results are shown in FIG. 19. The results indicate that both the first PROTAC product 1411-S3-1 and the second PROTAC product 1411-S3-2 can reduce the protein level of transcription factor STAT3 in a concentration-dependent manner, and the effect of the second PROTAC product 1411-S3-2 is better than that of the first PROTAC product 1411-S3-1. The half-maximal degradation concentrations (DC50) of the first PROTAC product 1411-S3-1 and the second PROTAC product 1411-S3-2 against STAT3 at the protein level were 262.5 nM and 136.3 nM, respectively, as shown in FIG. 19.

HeLa cells were incubated with 500 nM double-stranded transcription factor decoy oligonucleotide S3, AS1411, the first PROTAC product 1411-S3-1, the second PROTAC product 1411-S3-2, or AS1411+double-stranded transcription factor decoy oligonucleotide S3. After 6 h, cell samples were collected for protein extraction and subsequent western blot assay. The results are shown in FIG. 20. The results indicate that only the first PROTAC product 1411-S3-1 and the second PROTAC product 1411-S3-2 can reduce the protein level of transcription factor STAT3.

HeLa cells were incubated with 500 nM said first PROTAC product 1411-S3-1 or second PROTAC product 1411-S3-2. After 6 h, cell samples were collected, immunoprecipitation was performed, and the ubiquitination level of STAT3 was detected by western blot assay. The results are shown in FIG. 21. The results indicate that: both the first PROTAC product 1411-S3-1 and the second PROTAC product 1411-S3-2 can improve the ubiquitination of transcription factor STAT3.

HeLa cells were incubated with 500 nM said first PROTAC product 1411-S3-1 or second PROTAC product 1411-S3-2, and proteasome inhibitor MG132 was added. After 6 h, cell samples were collected for protein extraction and subsequent western blot assay. The results are shown in FIG. 22. The results indicate that proteasome inhibitor MG132 can block the protein degradation of transcription factor STAT3 by the first PROTAC product 1411-S3-1 and the second PROTAC product 1411-S3-2.

HeLa cells were transfected with si-NC or si-NCL and subsequently incubated with 500 nM said first PROTAC product 1411-S3-1 or second PROTAC product 1411-S3-2. After 6 h, cell samples were collected for protein extraction and subsequent western blot assay. The results are shown in FIG. 23. The results indicate that the first PROTAC product 1411-S3-1 and the second PROTAC product 1411-S3-2 cannot degrade transcription factor STAT3 after NCL is silenced.

HeLa cells were transfected with si-NC (SEQ ID NO: 16) or si-MDM2 (SEQ ID NO: 18) and subsequently incubated with 500 nM 1411-S3-1 or 1411-S3-2. After 6 h, cell samples were collected for protein extraction and subsequent western blot assay. The results are shown in FIG. 23. The results indicate that the first PROTAC product 1411-S3-1 and the second PROTAC product 1411-S3-2 cannot degrade transcription factor STAT3 after MDM2 is silenced.

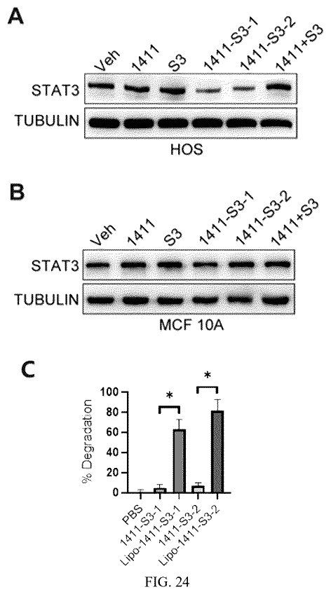

HOS osteosarcoma cells were incubated with 500 nM double-stranded transcription factor decoy oligonucleotide S3, AS1411, the first PROTAC product 1411-S3-1, the second PROTAC product 1411-S3-2, or AS1411+double-stranded transcription factor decoy oligonucleotide S3. After 6 h, cell samples were collected for protein extraction and subsequent western blot assay. The results are shown in FIG. 24A. The results indicate that only the first PROTAC product 1411-S3-1 and the second PROTAC product 1411-S3-2 can reduce the protein level of transcription factor STAT3.

MCF 10A human normal mammary epithelial cells were incubated with 500 nM double-stranded transcription factor decoy oligonucleotide S3, AS1411, the first PROTAC product 1411-S3-1, the second PROTAC product 1411-S3-2, or AS1411+double-stranded transcription factor decoy oligonucleotide S3. After 6 h, cell samples were collected for protein extraction and subsequent western blot assay. The results are shown in FIG. 24B. The results indicate that neither the first PROTAC product 1411-S3-1 nor the second PROTAC product 1411-S3-2 can reduce the protein level of transcription factor STAT3, indicating that the targeted degradation of STAT3 by the first PROTAC product 1411-S3-1 and the second PROTAC product 1411-S3-2 is tumor cell-specific. 40 μg/mL said first PROTAC product 1411-S3-1 or second PROTAC product 1411-S3-2 was encapsulated with a liposome (Lipo) (Lipofectamine 2000, purchased from Invitrogen, product code: 11668027) carrier to transfect MCF 10A human normal mammary epithelial cells. After 36 h, cell samples were collected for protein extraction and subsequent western blot assay. The results are shown in FIG. 24C. The results indicate that both the liposome-encapsulated first PROTAC product 1411-S3-1 and the liposome-encapsulated second PROTAC product 1411-S3-2 can reduce the protein level of transcription factor STAT3, indicating that both the first PROTAC product 1411-S3-1 and the second PROTAC product 1411-S3-2 penetrate cells depending on carriers, thereby degrading STAT3.

### Example 5

To verify the in vitro antitumor effects of the first PROTAC product 1411-S3-1 and the second PROTAC product 1411-S3-2 in Example 4, the following experiments were performed.

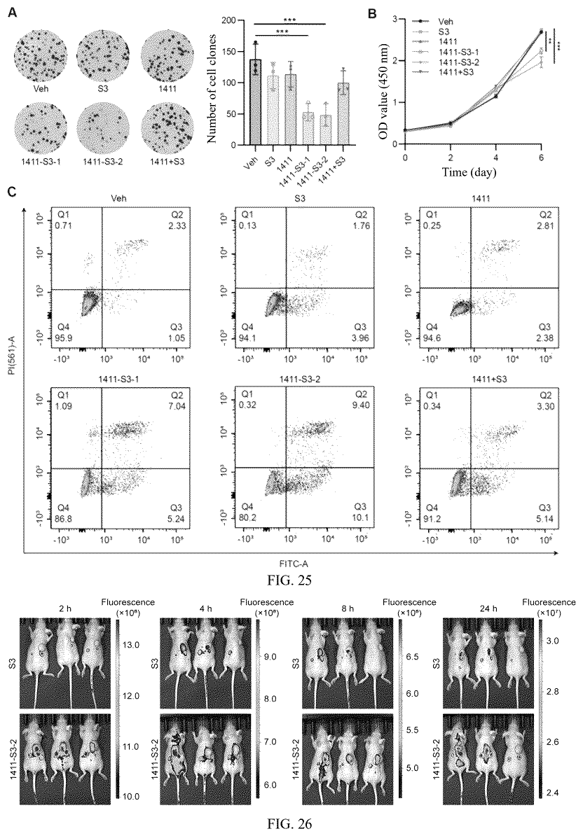

HeLa cells were seeded into a six-well plate for colony formation experiment. The medium containing 200 nM said first PROTAC product 1411-S3-1 or second PROTAC product 1411-S3-2 was renewed every day during the experiment. The number of formed colonies was observed after 10 days, and the results are shown in FIG. 25A. The results indicate that both the first PROTAC product 1411-S3-1 and the second PROTAC product 1411-S3-2 can inhibit the colony formation of HeLa cells, and the effect of the first PROTAC product 1411-S3-1 is better than that of the second PROTAC product 1411-S3-2.

HeLa cells were seeded into a 96-well cell culture plate for Cell Counting Kit-8 (CCK-8) experiment. The medium containing 500 nM said first PROTAC product 1411-S3-1 or second PROTAC product 1411-S3-2 was renewed every day during the experiment. After 6 days, the absorbance values at 450 nm were measured with a microplate reader, and the results are shown in FIG. 25B. The results indicate that both the first PROTAC product 1411-S3-1 and the second PROTAC product 1411-S3-2 can inhibit the cell proliferation of HeLa cells, and the effect of the second PROTAC product 1411-S3-2 is better than that of the first PROTAC product 1411-S3-1.

HeLa cells were seeded into a six-well plate for cell apoptosis experiment. The medium containing 200 nM said first PROTAC product 1411-S3-1 or second PROTAC product 1411-S3-2 was renewed every day during the experiment.

After 5 days, cell apoptosis analysis was performed using an Annexin V-FITC and PI double staining apoptosis detection kit, and the results are shown in FIG. 25C. The results indicate that both the first PROTAC product 1411-S3-1 and the second PROTAC product 1411-S3-2 can promote HeLa cell apoptosis, and the effect of the second PROTAC product 1411-S3-2 is better than that of the first PROTAC product 1411-S3-1.

### Example 6

To verify the in vivo tumor tissue-targeting effects of the first PROTAC product 1411-S3-1 and the second PROTAC product 1411-S3-2 in Example 4, the following experiments were performed.

Taking the second PROTAC product 1411-S3-2 as an example, HeLa cells were subcutaneously inoculated into BALB/c nude mice at a cell density of 1×10⁷ cells/mouse. Tumor growth was observed every day. When the tumors reached a volume of about 250 mm³, each mouse was injected with 5 nmol Cy5-labeled double-stranded transcription factor decoy oligonucleotide S3 or the second PROTAC product 1411-S3-2 via the tail vein. 2 h, 4 h, 8 h, and 24 h after injection, the mice were placed in a small animal in vivo imaging system to detect the in vivo distribution of the second PROTAC product 1411-S3-2. The results are shown in FIG. 26. The results indicate that compared with double-stranded transcription factor decoy oligonucleotide S3, the second PROTAC product 1411-S3-2 was highly aggregated at the tumor site.

The same experiment was performed using the first PROTAC product 1411-S3-1, and the experimental results were similar to those of the second PROTAC product 1411-S3-2.

### Example 7

To verify the in vivo antitumor effects and safety of the first PROTAC product 1411-S3-1 and the second PROTAC product 1411-S3-2 in Example 4, the following experiments were performed.

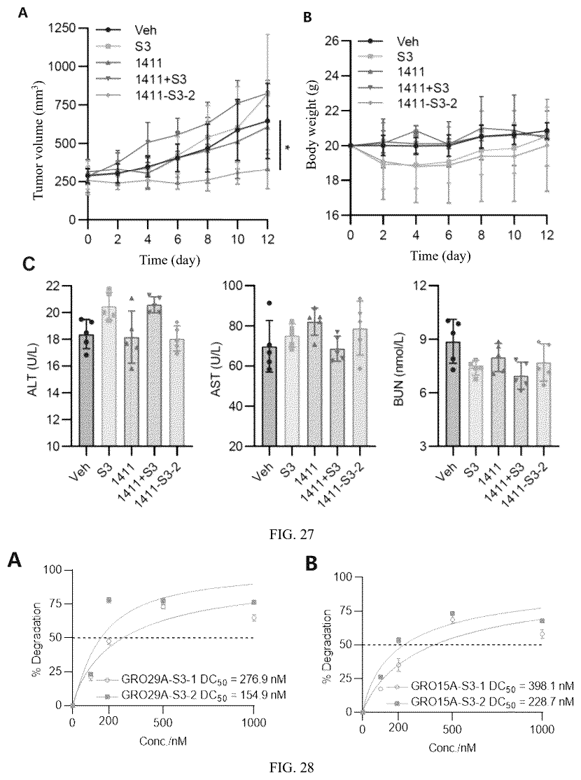

Taking the second PROTAC product 1411-S3-2 as an example, HeLa cells were subcutaneously inoculated into BALB/c nude mice at a cell density of 1×10⁷ cells/mouse. Tumor growth was observed every day. When the tumors reached a volume of about 250 mm³, a PBS buffer, double-stranded transcription factor decoy oligonucleotide S3, AS1411, the second PROTAC product 1411-S3-2, or AS1411+double-stranded transcription factor decoy oligonucleotide S3 was injected into the tail vein at an injection dose of 3 μmol/kg once every two days. After 12 days of administration, the tumor volume, body weight, and hepatotoxicity and nephrotoxicity were measured, and the results are shown in FIGS. 27A-27C. The results indicate that compared with PBS, double-stranded transcription factor decoy oligonucleotide S3, AS1411, or AS1411+double-stranded transcription factor decoy oligonucleotide S3, the second PROTAC product 1411-S3-2 can significantly inhibit tumor growth, see FIG. 27A. The body weight of the mice in the group injected with the second PROTAC product 1411-S3-2 showed no significant reduction, see FIG. 27B, and the hematological parameters (ALT and AST) of the liver and the parameter (BUN) of the kidney were both at normal levels, see FIG. 27C.

The same experiment was performed using the first PROTAC product 1411-S3-1, and the experimental results were similar to those of the second PROTAC product 1411-S3-2.

### Example 8: PROTAC Targeting STAT3

In this example, another PROTAC capable of degrading transcription factor STAT3 was prepared, wherein GRO29A was selected as the GRO. The sequence of GRO29A is set forth in SEQ ID NO: 2. Preferably, the conjugation procedures comprise: linking the antisense strand S3-R (SEQ ID NO: 6) of double-stranded transcription factor decoy oligonucleotide S3, a single-stranded DNA linker (5′-AAAAAA-3′), and GRO29A together by solid-phase synthesis, mixing with the sense strand S3-F (SEQ ID NO: 5) of double-stranded transcription factor decoy oligonucleotide S3 according to a molar ratio of 1:1, heating in a metal bath at 95° C. for 5 min, slowly annealing to room temperature, and completing the construction of the PROTAC by subjecting the nucleic acid sequences to base complementary pairing. The conjugation occurred at the 5′ end or 3′ end of GRO29A, forming two PROTAC products, i.e., GRO29A-S3-1 and GRO29A-S3-2. The sequences used in the aforementioned reactions can be synthesized by Sangon Biotech (Shanghai) Co., Ltd.

HeLa cells were incubated with GRO29A-S3-1 or GRO29A-S3-2 at concentrations of 0 nM, 100 nM, 200 nM, 500 nM, and 1000 nM. After 6 h, cell samples were collected to determine the half-maximal degradation concentration (DC50) of GRO29A-S3-1 or GRO29A-S3-2 against the STAT3 protein. The DC50 of GRO29A-S3-1 was 276.9 nM, and the DC50 of GRO29A-S3-2 was 154.9 nM, as shown in FIG. 28A.

### Example 9: PROTAC Targeting STAT3

In this example, another PROTAC capable of degrading transcription factor STAT3 was prepared, wherein GRO15A was selected as the GRO. The sequence of GRO15A is set forth in SEQ ID NO: 3. Preferably, the conjugation procedures comprise: linking the antisense strand S3-R (SEQ ID NO: 6) of double-stranded transcription factor decoy oligonucleotide S3, a single-stranded DNA linker (5′-AAAAAA-3′), and GRO15A together by solid-phase synthesis, mixing with the sense strand S3-F (SEQ ID NO: 5) of double-stranded transcription factor decoy oligonucleotide S3 according to a molar ratio of 1:1, heating in a metal bath at 95° C. for 5 min, slowly annealing to room temperature, and completing the construction of the PROTAC by subjecting the nucleic acid sequences to base complementary pairing. The conjugation occurred at the 5′ end or 3′ end of GRO15A, forming two PROTAC products, i.e., GRO15A-S3-1 and GRO15A-S3-2. The sequences used in the aforementioned reactions can be synthesized by Sangon Biotech (Shanghai) Co., Ltd.

HeLa cells were incubated with GRO15A-S3-1 or GRO15A-S3-2 at concentrations of 0 nM, 100 nM, 200 nM, 500 nM, and 1000 nM. After 6 h, cell samples were collected to determine the half-maximal degradation concentration (DC50) of GRO15A-S3-1 or GRO15A-S3-2 against the STAT3 protein. The DC50 of GRO15A-S3-1 was 398.1 nM, and the DC50 of GRO15A-S3-2 was 228.7 nM, as shown in FIG. 28B.

### Example 10: PROTAC Targeting STAT3

In this example, another PROTAC capable of degrading transcription factor STAT3 was prepared, wherein AT11 was selected as the GRO. The sequence of AT11 is set forth in SEQ ID NO: 4. Preferably, the conjugation procedures comprise: linking the antisense strand S3-R (SEQ ID NO: 6) of double-stranded transcription factor decoy oligonucleotide S3, a single-stranded DNA linker (5′-AAAAAA-3′), and AT11 together by solid-phase synthesis, mixing with the sense strand S3-F (SEQ ID NO: 5) of double-stranded transcription factor decoy oligonucleotide S3 according to a molar ratio of 1:1, heating in a metal bath at 95° C. for 5 min, slowly annealing to room temperature, and completing the construction of the PROTAC by subjecting the nucleic acid sequences to base complementary pairing. The conjugation occurred at the 5′ end or 3′ end of AT11, forming two PROTAC products, i.e., AT11-S3-1 and AT11-S3-2. The sequences used in the aforementioned reactions can be synthesized by Sangon Biotech (Shanghai) Co., Ltd.

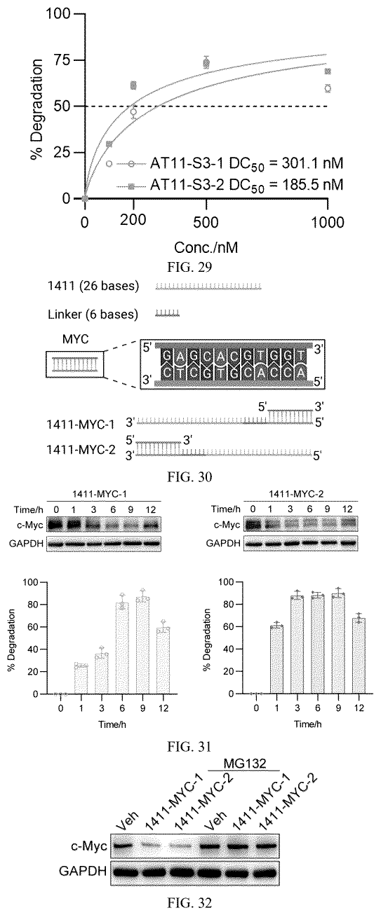

HeLa cells were incubated with AT11-S3-1 or AT11-S3-2 at concentrations of 0 nM, 100 nM, 200 nM, 500 nM, and 1000 nM. After 6 h, cell samples were collected to determine the half-maximal degradation concentration (DC50) of AT11-S3-1 or AT11-S3-2 against the STAT3 protein. The DC50 of AT11-S3-1 was 301.1 nM, and the DC50 of AT11-S3-2 was 185.5 nM, as shown in FIG. 29.

### Example 11: PROTAC Targeting c-Myc

In this example, a PROTAC capable of degrading transcription factor c-Myc was prepared. As a transcription factor with a wide range of effects, c-Myc is a key protein for regulating cell fate, but is also one of the recognized difficult-to-drug targets. Therefore, the means of regulating and intervening c-Myc have remained as one of the forefront and hot directions in international competition.

In this example, the PROTAC capable of degrading transcription factor c-Myc was formed by conjugating a novel recruitment element GRO of an ubiquitin ligase MDM2 and a ligand of c-Myc via a linker. The GRO includes DNA aptamer AS1411 and a derivative or analog thereof (e.g., GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M). The PROTAC can effectively degrade c-Myc. In this example, DNA aptamer AS1411 was selected as the GRO.

The ligand of c-Myc includes small molecule compounds, oligonucleotides (e.g., nucleic acid aptamers, etc.), oligopeptides, polypeptides, proteins (e.g., antibodies), and the like. In a preferred example of the present disclosure, the ligand of c-Myc comprises a double-stranded transcription factor decoy oligonucleotide, wherein the sense strand sequence MYC-F of the oligonucleotide is set forth in SEQ ID NO: 7, and the antisense strand sequence MYC-R of the oligonucleotide is set forth in SEQ ID NO: 8. The linker of the PROTAC includes a single-stranded DNA oligonucleotide with 6 A bases. The double-stranded transcription factor decoy oligonucleotide that specifically recognizes c-Myc was conjugated to AS1411 via the single-stranded DNA oligonucleotide linker to form the PROTAC.

The conjugation procedures comprise: linking the antisense strand MYC-R (SEQ ID NO: 8) of the double-stranded transcription factor decoy oligonucleotide, a single-stranded DNA linker (5′-AAAAAA-3′), and AS1411 together by solid-phase synthesis, mixing with the sense strand MYC-F (SEQ ID NO: 7) of the double-stranded transcription factor decoy oligonucleotide according to a molar ratio of 1:1, heating in a metal bath at 95° C. for 5 min, slowly annealing to room temperature, and completing the construction of the PROTAC by subjecting the nucleic acid sequences to base complementary pairing. The conjugation occurred at the 5′ end or 3′ end of AS1411, forming two PROTAC products, i.e., a first PROTAC product 1411-MYC-1 and a second PROTAC product 1411-MYC-2, as shown in FIG. 30.

In a further preferred example of the present disclosure, a derivative or analog of AS1411 could also be used to conjugate to a double-stranded transcription factor decoy oligonucleotide of c-Myc via a single-stranded DNA oligonucleotide with 6 A bases to form a PROTAC product, and the procedures are the same as those with AS1411 being used and will not be repeated herein.

The following was performed to further verify the ability of the novel PROTAC product prepared based on AS1411 for c-Myc, i.e., AS1411-MYC-1 or AS1411-MYC-2, to degrade c-Myc at the protein level, as detailed below.

(1) HeLa cells were incubated with 200 nM AS1411-MYC-1 or AS1411-MYC-2, and corresponding cell samples were collected at time gradients of 0 h, 1 h, 3 h, 6 h, 9 h, and 12 h for protein extraction and subsequent western blot assay to detect protein degradation. The results are shown in FIG. 31. The results indicate that AS1411-MYC-1 and AS1411-MYC-2 can significantly reduce the protein level of c-Myc in a time-dependent manner.

(2) HeLa cells were incubated with 500 nM AS1411-MYC-1 or AS1411-MYC-2 for 9 h, and proteasome inhibitor MG132 was added. Cell samples were collected for protein extraction and subsequent western blot assay. The results are shown in FIG. 32. The results indicate that proteasome inhibitor MG132 can block the protein degradation of c-Myc by AS1411-MYC-1 and AS1411-MYC-2.

(3) HeLa cells were transfected with si-NC or si-NCL and subsequently incubated with 500 nM AS1411-MYC-1 or AS1411-MYC-2 for 9 h. Cell samples were collected for protein extraction and subsequent western blot assay. The results are shown in FIG. 33. The results indicate that AS1411-MYC-1 and AS1411-MYC-2 cannot degrade c-Myc after NCL is silenced.

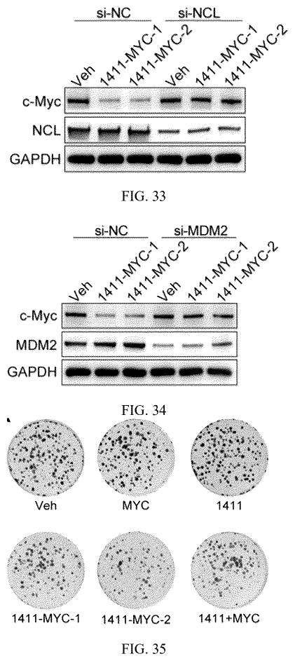

(4) HeLa cells were transfected with si-NC or si-MDM2 and subsequently incubated with 500 nM AS1411-MYC-1 or AS1411-MYC-2 for 9 h. Cell samples were collected for protein extraction and subsequent western blot assay. The results are shown in FIG. 34. The results indicate that AS1411-MYC-1 and AS1411-MYC-2 cannot degrade c-Myc after MDM2 is silenced.

To verify the in vitro antitumor effect of AS1411-MYC-1 or AS1411-MYC-2, the following experiments were performed.

(5) HeLa cells were seeded into a six-well plate for colony formation experiment. The medium containing 200 nM AS1411-MYC-1 or AS1411-MYC-2 was renewed every day during the experiment. The number of formed colonies was observed after 10 days, and the results are shown in FIG. 35. The results indicate that both AS1411-MYC-1 and AS1411-MYC-2 can inhibit the colony formation of HeLa cells.

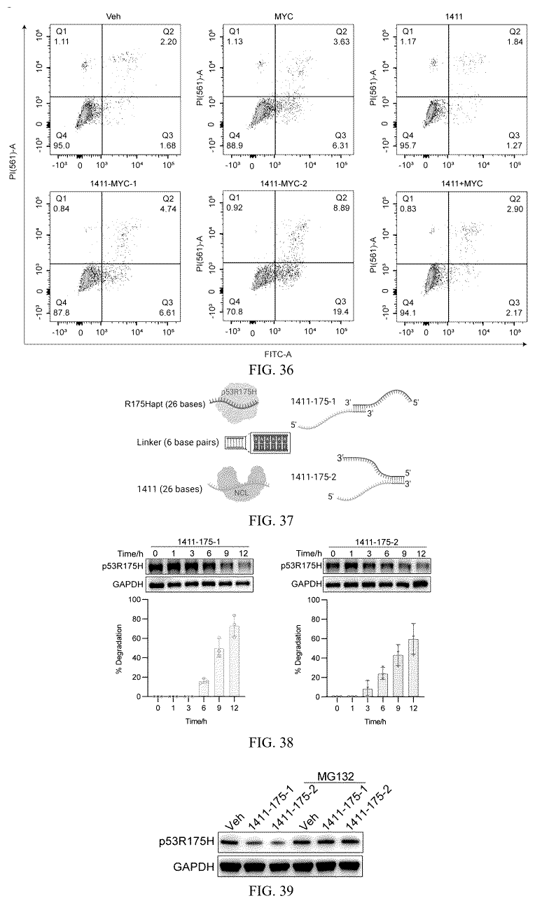

(6) HeLa cells were seeded into a six-well plate for cell apoptosis experiment. The medium containing 200 nM AS1411-MYC-1 or AS1411-MYC-2 was renewed every day during the experiment. After 5 days, cell apoptosis analysis was performed using an Annexin V-FITC and PI double staining apoptosis detection kit, and the results are shown in FIG. 36. The results indicate that both AS1411-MYC-1 and AS1411-MYC-2 can promote HeLa cell apoptosis.

In a further preferred example of the present disclosure, a derivative or analog of AS1411 was used to conjugate to a double-stranded transcription factor decoy oligonucleotide of c-Myc via a single-stranded DNA oligonucleotide with 6 A bases to form a PROTAC product. The experiments described above were performed and all achieved similar results.

### Example 12: PROTAC Targeting p53-R175H

Among the currently known tumor-associated genes, TP53 is one of the most studied and important cancer suppressor genes. The p53 protein encoded by TP53 is a stress-responsive protein and can counteract adverse factors such as genotoxic stress, oncogene signal activation, and DNA damage by regulating gene transcription. Recent genome-wide association studies suggest that TP53 is one of the most frequently mutated genes in human malignancies, with a mutation frequency reaching up to 50%. The most common type of TP53 gene mutation is a missense mutation occurring in the DNA-binding domain, including hotspot mutations R175H, G245S, R248W/Q, R249S, R273C/H, and R282W. Unlike other cancer suppressor genes that merely lose their cancer suppressor function when mutated, the mutated TP53 gene exhibits a unique property, that is, the mutated gene acquires a new function of cancer promotion. Among these hotspot mutations of the TP53 gene, R175H is the most prevalent mutation, characterized by substitution of arginine with histidine at position 175 in the encoded p53 protein. p53-R175H not only loses the transactivation function of wild-type p53 and is prone to aggregation, but also acquires oncogenic functions through interactions with multiple proteins. Consequently, p53-R175H represents an important target in precision tumor medicine treatment. As a nuclear transcription factor, p53 lacks typical druggable features and thus has long been considered an undruggable target. For decades, functional restoration of mutant p53 in tumors has been pursued as a therapeutic strategy. However, these efforts have failed, with few drugs that target p53 mutants advancing to late-stage clinical trials, and none achieving approval for clinical use to date.

The objective of the example of the present disclosure is to provide a PROTAC capable of degrading p53-R175H.

The PROTAC capable of degrading p53-R175H was formed by conjugating a novel recruitment element GRO of ubiquitin ligase MDM2 and a ligand of p53-R175H via a linker. The GRO includes DNA aptamer AS1411 and a derivative or analog thereof (e.g., GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M). The PROTAC can effectively degrade p53-R175H. In this example, DNA aptamer AS1411 was selected as the GRO.

The ligand of p53-R175H includes small molecule compounds, oligonucleotides (e.g., nucleic acid aptamers, etc.), oligopeptides, polypeptides, proteins (e.g., antibodies), and the like. In a preferred example of the present disclosure, a ligand of p53-R175H includes RNA aptamer R175Hapt of p53-R175H, which has the sequence set forth in SEQ ID NO: 9.

The linker of the PROTAC includes a DNA/RNA hybrid double-stranded oligonucleotide with β-30 A-T pairs, and more preferably, the linker of the PROTAC includes a DNA/RNA hybrid double-stranded oligonucleotide with 6 A-T pairs, wherein the DNA single strand in the DNA/RNA hybrid double-stranded oligonucleotide is a sequence with 6 T bases, and the RNA single strand in the DNA/RNA hybrid double-stranded oligonucleotide is a sequence with 6 A bases.

The preparation method for the PROTAC capable of degrading p53-R175H was as follows: The 3′ end of AS1411 was linked to the DNA single strand of the DNA/RNA hybrid double-stranded oligonucleotide by solid-phase synthesis to form a first conjugate product;
the 5′ end or 3′ end of R175Hapt was linked to the RNA single strand of the DNA/RNA hybrid double-stranded oligonucleotide by solid-phase synthesis to form a second conjugate product;
the first conjugate product and the second conjugate product were separately heated in a metal bath at 95° C. for 5 min, cooled to 37° C., left to stand for 30 min, and mixed in a molar ratio of 1:1, so that the novel PROTAC for p53-R175H, i.e., AS1411-175-1 or AS1411-175-2, was constructed by base complementary pairing of the DNA/RNA hybrid double-stranded oligonucleotide, as shown in FIG. 37.

In a further preferred example of the present disclosure, a derivative or analog of AS1411 could also be used to conjugate to R175Hapt via a DNA/RNA hybrid double-stranded oligonucleotide to form a PROTAC product, and the procedures are the same as those with AS1411 being used and will not be repeated herein.

The following was performed to further verify the ability of the PROTAC capable of degrading p53-R175H prepared based on AS1411, i.e., AS1411-175-1 or AS1411-175-2, to degrade p53-R175H at the protein level, as detailed below.

(1) H1299 human non-small cell lung cancer cells expressing p53-R175H were incubated with 200 nM AS1411-175-1 or AS1411-175-2, and corresponding cell samples were collected at time gradients of 0 h, 1 h, 3 h, 6 h, 9 h, and 12 h for protein extraction and subsequent western blot assay to detect protein degradation. The results are shown in FIG. 38. The results indicate that AS1411-175-1 and AS1411-175-2 can significantly reduce the protein level of p53-R175H in a time-dependent manner.

(2) H1299 expressing p53-R175H was incubated with 500 nM AS1411-175-1 or AS1411-175-2 for 12 h, and proteasome inhibitor MG132 was added. Cell samples were collected for protein extraction and subsequent western blot assay. The results are shown in FIG. 39. The results indicate that proteasome inhibitor MG132 can block the protein degradation of p53-R175H by AS1411-175-1 and AS1411-175-2.

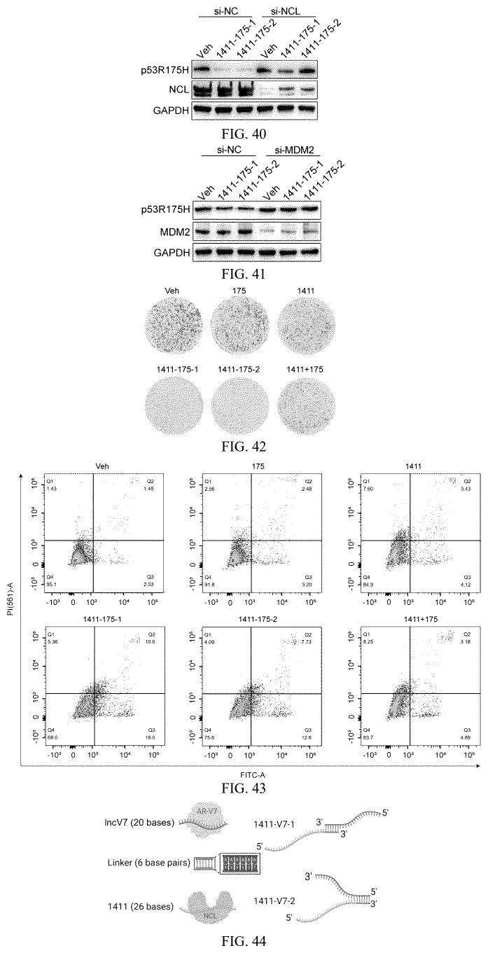

(3) H1299 expressing p53-R175H was transfected with si-NC or si-NCL and subsequently incubated with 500 nM AS1411-175-1 or AS1411-175-2 for 12 h. Cell samples were collected for protein extraction and subsequent western blot assay. The results are shown in FIG. 40. The results indicate that AS1411-175-1 and AS1411-175-2 cannot degrade p53-R175H after NCL is silenced.

(4) H1299 expressing p53-R175H was transfected with si-NC or si-MDM2 and subsequently incubated with 500 nM AS1411-175-1 or AS1411-175-2 for 12 h. Cell samples were collected for protein extraction and subsequent western blot assay. The results are shown in FIG. 41. The results indicate that AS1411-175-1 and AS1411-175-2 cannot degrade p53-R175H after MDM2 is silenced.

To verify the in vitro antitumor effects of AS1411-175-1 and AS1411-175-2, the following experiments were performed.

(5) H1299 expressing p53-R175H was seeded into a six-well plate for colony formation experiment. The medium containing 200 nM AS1411-175-1 or AS1411-175-2 was renewed every day during the experiment. The number of formed colonies was observed after 10 days, and the results are shown in FIG. 42. The results indicate that both AS1411-175-1 and AS1411-175-2 can inhibit the colony formation of H1299 cells.

(6) H1299 tumor cells expressing p53-R175H were seeded into a six-well plate for cell apoptosis experiment. The medium containing 200 nM AS1411-175-1 or AS1411-175-2 was renewed every day during the experiment. After 3 days, cell apoptosis analysis was performed using an Annexin V-FITC and PI double staining apoptosis detection kit, and the results are shown in FIG. 43. The results indicate that both AS1411-175-1 and AS1411-175-2 can promote H1299 cell apoptosis.

In a further preferred example of the present disclosure, a derivative or analog of AS1411 was used to conjugate to R175Hapt via a DNA/RNA hybrid double-stranded oligonucleotide to form a PROTAC product. The experiments described above were performed and all achieved similar results.

### Example 13: PROTAC Targeting AR-V7

Androgen receptor (AR), a member of the steroid receptor subfamily within the nuclear receptor superfamily, functions as a transcription factor regulating eukaryotic gene expression. It plays critical roles in the development and homeostasis of reproductive, musculoskeletal, cardiovascular, nervous, immune, and hematopoietic systems. In its inactive state, AR resides in the cytoplasm and binds to heat shock proteins while preventing misfolding and maintaining the 3D protein structure during the event of cellular stress. Upon binding to androgen molecules, AR undergoes activation, and this promotes the homodimerization and nuclear translocation of AR. Subsequently, AR binds to androgen response elements, leading to the activation and transcription of a variety of downstream genes, including various cancer-associated signaling pathways such as PI3K/AKT. In addition, AR also has non-genomic functions that can affect the growth, migration, metastasis, and apoptosis of cells. Androgen deprivation therapy is the main clinical treatment regimen for patients with prostate cancer. However, as patients progress to metastatic castration-resistant prostate cancer, most prostate cancers eventually develop androgen independence and resistance to androgen deprivation therapy. One of the key mechanisms underlying this resistance involves the constitutively activated androgen receptor splice variant 7 (AR-V7), which lacks the ligand binding domain due to splicing, enabling it to activate the expression of target genes in the absence of androgen. AR-V7 is an important resistance target. Researchers are working to develop therapeutic strategies for targeting AR-V7.

Bavdegalutamide (namely ARV-110, developed by Arvinas), the first PROTAC protein degrader with specificity for the androgen receptor in the world to enter clinical trials, selectively targets and degrades the AR protein, offering a treatment for metastatic castration-resistant prostate cancer. Recent clinical trial data show that ARV-110 exhibits potent antitumor activity and clinical benefit in the treatment of patients with metastatic castration-resistant prostate cancer carrying AR T878X and/or H875Y (T878X=T878A or T878S) mutations. Furthermore, in tumor patients who do not carry the AR T878X/H875Y mutations, ARV-110 treatment can also lead to reductions in prostate-specific antigen indexes and tumor regression. However, ARV-110 cannot degrade AR-V7.

The objective of the example of the present disclosure is to provide a PROTAC capable of degrading AR-V7. The PROTAC was formed by conjugating a novel recruitment element GRO of ubiquitin ligase MDM2 and a ligand of AR-V7 via a linker. The GRO includes DNA aptamer AS1411 and a derivative or analog thereof (e.g., GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M). The PROTAC can effectively degrade AR-V7. In this example, AS1411 was selected as the GRO.

The ligand of AR-V7 is RNA oligonucleotide lncRV having the sequence set forth in SEQ ID NO: 10 or lncV7 having the sequence set forth in SEQ ID NO: 11.

The linker of the PROTAC includes a DNA/RNA hybrid double-stranded oligonucleotide with 3-30 A-T pairs, and more preferably, the linker of the PROTAC includes a DNA/RNA hybrid double-stranded oligonucleotide with 6 A-T pairs, wherein the DNA single strand in the DNA/RNA hybrid double-stranded oligonucleotide is a sequence with 6 T bases, and the RNA single strand in the DNA/RNA hybrid double-stranded oligonucleotide is a sequence with 6 A bases.

The preparation method for the PROTAC capable of degrading AR-V7 was as follows: The 3′ end of AS1411 was linked to the DNA single strand of the DNA/RNA hybrid double-stranded oligonucleotide by solid-phase synthesis to form a first conjugate product;
the 5′ end or 3′ end of lncV7 was linked to the RNA single strand of the DNA/RNA hybrid double-stranded oligonucleotide by solid-phase synthesis to form a second conjugate product;
the first conjugate product and the second conjugate product were separately heated in a metal bath at 95° C. for 5 min, cooled to 37° C., left to stand for 30 min, and mixed in a molar ratio of 1:1, so that the PROTAC capable of degrading AR-V7, i.e., AS1411-V7-1 or AS1411-V7-2, was constructed by base complementary pairing of the DNA/RNA hybrid double-stranded oligonucleotide, as shown in FIG. 44.

In a further preferred example of the present disclosure, a derivative or analog of AS1411 could also be used to conjugate to lncV7 via a DNA/RNA hybrid double-stranded oligonucleotide to form a PROTAC product, and the procedures are the same as those with AS1411 being used and will not be repeated herein.

The following was performed to further verify the ability of the PROTAC capable of degrading AR-V7 prepared based on AS1411, i.e., AS1411-V7-1 or AS1411-V7-2, to degrade AR-V7 at the protein level, as detailed below.

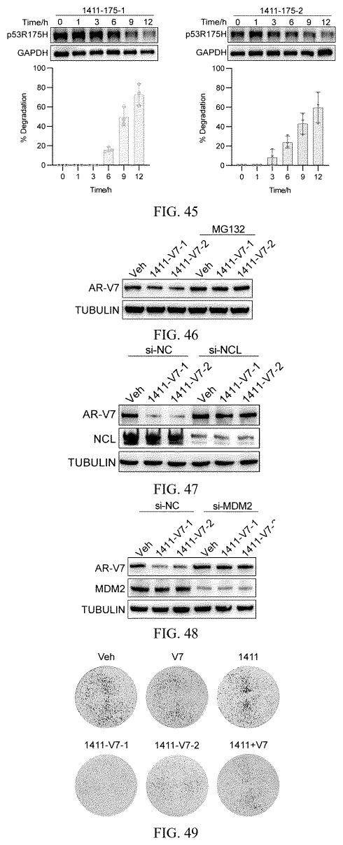

(1) 22Rv1 prostate cancer cells were incubated with 200 nM AS1411-V7-1 or AS1411-V7-2, and corresponding cell samples were collected at time gradients of 0 h, 3 h, 6 h, 9 h, and 12 h for protein extraction and subsequent western blot assay to detect protein degradation. The results are shown in FIG. 45. The results indicate that AS1411-V7-1 and AS1411-V7-2 can significantly reduce the protein level of AR-V7 in a time-dependent manner.

(2) 22Rv1 prostate cancer cells were incubated with 500 nM AS1411-V7-1 or AS1411-V7-2 for 12 h, and proteasome inhibitor MG132 was added. Cell samples were collected for protein extraction and subsequent western blot assay.

The results are shown in FIG. 46. The results indicate that proteasome inhibitor MG132 can block the protein degradation of AR-V7 by AS1411-V7-1 and AS1411-V7-2.

(3) 22Rv1 prostate cancer cells were transfected with si-NC or si-NCL and subsequently incubated with 500 nM AS1411-V7-1 or AS1411-V7-2 for 12 h. Cell samples were collected for protein extraction and subsequent western blot assay. The results are shown in FIG. 47. The results indicate that AS1411-V7-1 and AS1411-V7-2 cannot degrade AR-V7 after NCL is silenced.

(4) 22Rv1 prostate cancer cells were transfected with si-NC or si-MDM2 and subsequently incubated with 500 nM AS1411-V7-1 or AS1411-V7-2 for 12 h. Cell samples were collected for protein extraction and subsequent western blot assay. The results are shown in FIG. 48. The results indicate that AS1411-V7-1 and AS1411-V7-2 cannot degrade AR-V7 after MDM2 is silenced.

To verify the in vitro antitumor effects of AS1411-V7-1 and AS1411-V7-2, the following experiments were performed.

(5) 22Rv1 prostate cancer cells were seeded into a six-well plate for colony formation experiment. The medium containing 500 nM AS1411-V7-1 or AS1411-V7-2 was renewed every day during the experiment. The number of formed colonies was observed after 10 days, and the results are shown in FIG. 49. The results indicate that both AS1411-V7-1 and AS1411-V7-2 can inhibit the colony formation of 22Rv1 cells.

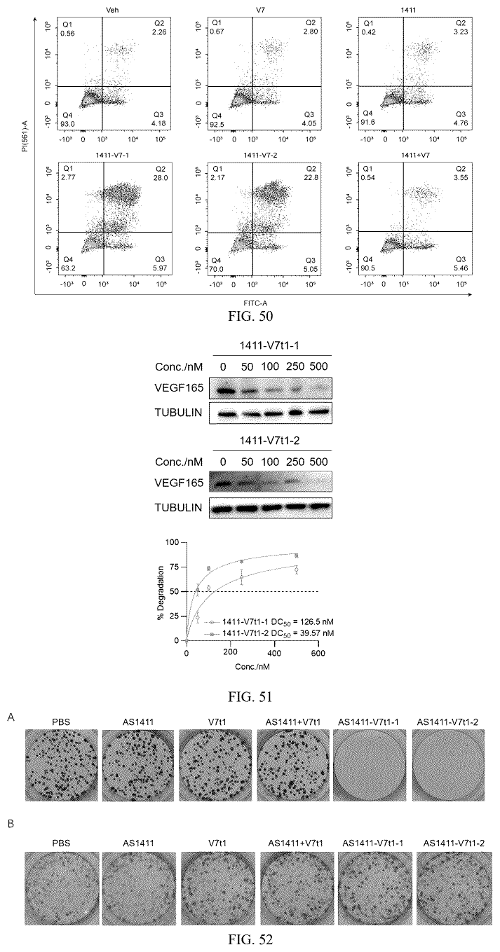

(6) 22Rv1 prostate cancer cells were seeded into a six-well plate for cell apoptosis experiment. The medium containing 500 nM AS1411-V7-1 or AS1411-V7-2 was renewed every day during the experiment. After 6 days, cell apoptosis analysis was performed using an Annexin V-FITC and PI double staining apoptosis detection kit, and the results are shown in FIG. 50. The results indicate that both AS1411-V7-1 and AS1411-V7-2 can promote 22Rv1 cell apoptosis.

In a further preferred example of the present disclosure, a derivative or analog of AS1411 was used to conjugate to lncV7 via a DNA/RNA hybrid double-stranded oligonucleotide to form a PROTAC product. The experiments described above were performed and all achieved similar results.

In a further preferred example of the present disclosure, AS1411 or a derivative or analog of AS1411 was used to conjugate to lncRV via a DNA/RNA hybrid double-stranded oligonucleotide to form a PROTAC product. The experiments described above were performed and all achieved similar results.

### Example 14: PROTAC Targeting VEGF

Vascular endothelial growth factor (VEGF) is an important protein secreted by epithelial cells, tumor cells, macrophages, and the like. It has a variety of functions, including stimulating angiogenesis, increasing vascular permeability, enhancing tumor invasion and survival, and inhibiting the antitumor response of regulatory T cells.

VEGF has different splice variants, with VEGF165 being the most common form.

The example of the present disclosure provides a PROTAC capable of degrading VEGF165. The PROTAC was formed by conjugating a novel recruitment element GRO of ubiquitin ligase MDM2 to a ligand of VEGF165 via a linker. The GRO includes DNA aptamer AS1411 and a derivative or analog thereof (e.g., GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M). The PROTAC can effectively degrade VEGF165. In this example, AS1411 was selected as the GRO.

The ligand of VEGF165 includes small molecule compounds, oligonucleotides, nucleic acid aptamers, oligopeptides, polypeptides, proteins (e.g., antibodies), and the like. In this example, the ligand of VEGF165 includes single-stranded DNA aptamer V7t1 having the sequence set forth in SEQ ID NO: 12.

In this example, the linker includes a single-stranded DNA oligonucleotide with 6 A bases. The DNA aptamer V7t1 that specifically recognizes VEGF165 was conjugated to AS1411 via the single-stranded DNA oligonucleotide linker to form the PROTAC.

Preferably, the conjugation procedure for the PROTAC capable of degrading VEGF165 comprises: linking the DNA aptamer V7t1 of VEGF165, the single-stranded DNA linker, and AS1411 together according to a ratio of 1:1:1 by solid-phase synthesis to complete the construction of the PROTAC. The linkage occurred at the 5′ end or 3′ end of AS1411, forming two PROTAC products, i.e., a first PROTAC product 1411-V7t1-1 and a second PROTAC product 1411-V7t1-2.

In a further preferred example of the present disclosure, a derivative or analog of AS 1411 could also be used to conjugate to the DNA aptamer V7t1 of VEGF165 via a single-stranded DNA oligonucleotide with 6 A bases to form a PROTAC product, and the procedures are the same as those with AS 1411 being used and will not be repeated herein.

HeLa cervical cancer cells were incubated with 1411-V7t1-1 or 1411-V7t1-2 at concentrations of 0 nM, 50 nM, 100 nM, 250 nM, 500 nM, and 1000 nM. After 12 h, cell samples were collected to determine the half-maximal degradation concentration (DC50) of 1411-V7t1-1 or 1411-V7t1-2 against the VEGF165 protein. The DC50 of 1411-V7t1-1 was 130.6 nM, and the DC50 of 1411-V7t1-2 was 32.31 nM, as shown in FIG. 51.

To verify the in vitro antitumor effect of 1411-V7t1-1 or 1411-V7t1-2, the following experiments were performed.

HeLa cervical cancer cells were seeded into a six-well plate for colony formation experiment. The medium containing 500 nM 1411-V7t1-1 or 1411-V7t1-2 was renewed every 3 days during the experiment. The number of formed colonies was observed after 7 days, and the results are shown in FIG. 52A. The results indicate that both 1411-V7t1-1 and 1411-V7t1-2 can inhibit the colony formation of HeLa cells.

MCF 10A human normal mammary epithelial cells were seeded into a six-well plate for colony formation experiment.

The medium containing 500 nM 1411-V7t1-1 or 1411-V7t1-2 was renewed every 3 days during the experiment. The number of formed colonies was observed after 6 days, and the results are shown in FIG. 52B. The results indicate that neither 1411-V7t1-1 nor 1411-V7t1-2 can inhibit the colony formation of MCF 10A cells, indicating that 1411-V7t1-1 and 1411-V7t1-2 have tumor cell specificity.

In a further preferred example of the present disclosure, a derivative or analog of AS1411 was used to conjugate to the DNA aptamer V7t1 of VEGF165 via a single-stranded DNA oligonucleotide with 6 A bases to form a PROTAC product. The experiments described above were performed and all achieved similar results.

### Example 15: PROTAC Targeting MDM2

MDM2 holds a special position in the development of PROTACs. In one aspect, it serves as one of the four E3 ubiquitin ligases commonly used in PROTACs (the other three are CRBN, VHL, and IAP, respectively) and is recruited by ligands in PROTACs for ubiquitination and degradation of a variety of target proteins and disease treatment. In another aspect, MDM2 is also a protooncogene that is highly expressed in various tumors. As a key negative regulator of tumor suppressor p53, it plays an important role in the development and progression of tumors.

Consequently, MDM2 is also often recognized by PROTACs as a target protein for ubiquitination and degradation for tumor treatment.

The objective of the present disclosure is to provide a PROTAC capable of degrading MDM2. The PROTAC is formed by conjugating a novel recruitment element GRO of MDM2 to a ligand of E3 ubiquitin ligase via a linker.

The GRO includes DNA aptamer AS1411 and a derivative or analog thereof (e.g., GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M). In this example, AS1411 was selected as the GRO. The PROTAC can effectively degrade MDM2.

The E3 ubiquitin ligase ligand includes small molecule compounds, oligonucleotides, nucleic acid aptamers, oligopeptides, polypeptides, and proteins (e.g., antibodies). In a further preferred example of the present disclosure, the E3 ubiquitin ligase is VHL, and the E3 ubiquitin ligase ligand is small molecule VH032.

In a further preferred example of the present disclosure, the linker is an alkyl chain.

The specific synthesis process of conjugating AS1411 to the small molecule ligand VH032 of E3 ubiquitin ligase VHL via a linker to form a PROTAC degrader for MDM2 is shown below. In this preferred example, the PROTAC capable of degrading MDM2 is AS1411-VH032, and the specific preparation process was as follows:

### (1) Synthesis of Compound B

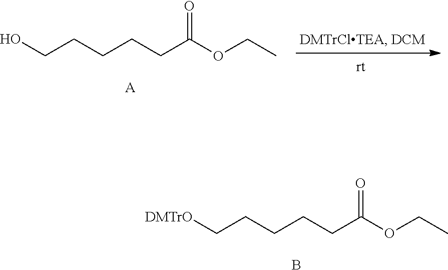

4,4′-Dimethoxytrityl chloride (DMTr-Cl, 5 g, 14.98 mmol) was dissolved in dichloromethane (DCM, 100 mL), and triethylamine (TEA, 3.16 g, 31.21 mmol) and compound A (2 g, 12.48 mmol) were then added. The mixture described above was stirred at room temperature for 1 h and monitored using thin-layer chromatography. The mixture described above was diluted with DCM (100 mL) and washed separately with water and saturated NaHCO₃. The combined organic layers were dried over Na₂SO₄, filtered, and concentrated under vacuum to give compound B (5 g, crude product) as a white solid, which was used in the next reaction without purification.

LCMS: No MS, Rt=2.112 min, purity: 97.3% (214 nm)

### (2) Synthesis of Compound INT-1

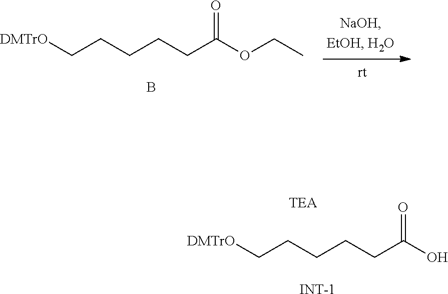

Compound B (5 g, 10.81 mmol) was dissolved in ethanol (40 mL), and a 6 mol/L aqueous NaOH solution (20 mL) was added. The mixture described above was stirred at room temperature for 2 h. The reaction described above was monitored using a liquid chromatograph-mass spectrometer. After the reaction was completed, the mixture was concentrated to remove ethanol, adjusted to pH 6 with citric acid (5%) at 0° C., extracted with DCM (100 mL×3), and washed with saturated brine (3×50 mL). The combined organic layers were dried over Na₂SO₄, filtered, concentrated, purified by flash column chromatography, and eluted with DCM/methanol/TEA (10:1:0.004) to give compound INT-1 (4.7 g, yield: 81%) as a white solid.

LCMS: (M−H)⁻=433.2, Rt=1.183 min, purity: 99.9%

¹HNMR (400 MHz, CDCl₃) δ 7.43 (m, 2H), 7.30 (m, 6H), 7.19 (m, 1H), 6.81 (m, 4H), 3.78 (s, 6H), 3.03 (t, J=6.6 Hz, 2H), 2.25 (m, 2H), 1.61 (m, 4H), 1.40 (m, 2H)

### (3) Synthesis of Compound 2

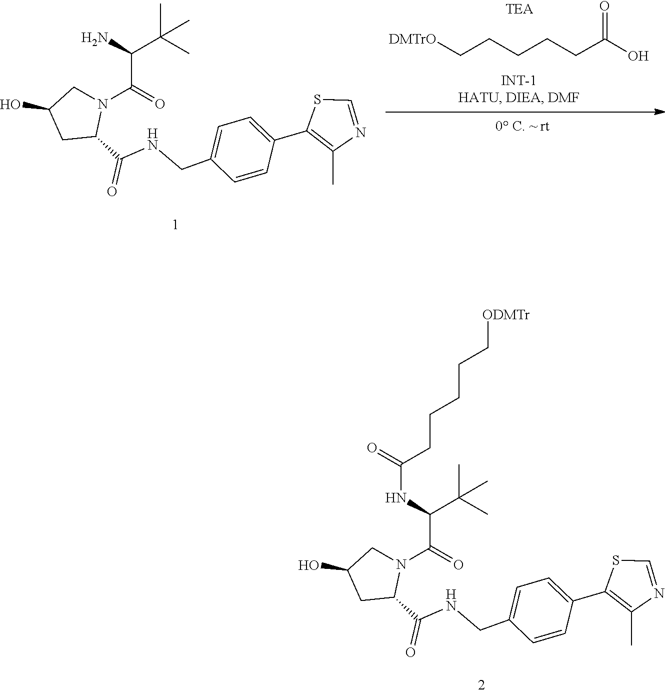

INT-1 (558 mg, 1.28 mmol) was dissolved in dimethylformamide (DMF, 10 mL), and O-(7-azabenzotriazol-1-yl)-N,N,N′,N′-tetramethyluronium hexafluorophosphate (HATU, 814 mg, 2.14 mmol) was added at 0° C. under a nitrogen atmosphere. The mixture described above was stirred at room temperature for 30 min, and compound 1 (500 mg, 1.07 mmol) and N,N-diisopropylethylamine (DIEA, 692 mg) were slowly added at 0° C. The mixture described above was stirred at room temperature overnight and monitored by a liquid chromatograph-mass spectrometer. The mixture described above was diluted with water and extracted with ethyl acetate (EA, 3×10 mL). The combined organic layers were dried over Na₂SO₄, filtered, and evaporated under reduced pressure to remove the solvent to give a residue. The residue was purified by flash chromatography and eluted with ethyl acetate/polyethylene (PE) (0-30%) to give compound 2 (900 mg, yield: 91%) as a white solid.

LCMS: (M−H)⁻=845.2.2, Rt=1.9 min, purity: 90.0%

¹HNMR (400 MHz, MeOD) δ 8.86 (s, 1H), 7.46 (m, 2H), 7.42-7.38 (m, 4H), 7.26 (m, 6H), 7.17 (m, 1H), 6.83 (m, 4H), 4.61 (s, 1H), 4.56 (m, 1H), 4.51 (m, 2H), 4.34 (m, 1H), 3.89 (m, 1H), 3.83-3.77 (m, 1H), 3.75 (m, 6H), 3.04 (m, 2H), 2.74 (s, 2H), 2.46 (s, 3H), 2.19 (m, 1H), 2.09 (m, 1H), 1.66-1.53 (m, 4H), 1.41 (m, 2H), 1.00 (s, 9H).

### (4) Synthesis of Compound 3

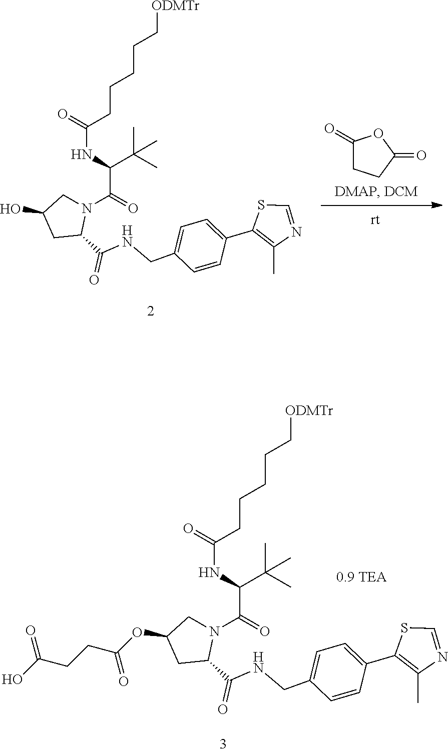

Compound 2 (900 mg, 1.06 mmol) was dissolved in DCM (30 mL), and 4-dimethylaminopyridine (DMAP, 390 mg, 3.18 mmol) and succinic anhydride (160 mg, 1.60 mmol) were added. The mixture described above was stirred at room temperature for 12 h under a nitrogen atmosphere and monitored by a liquid chromatograph-mass spectrometer.

The mixture described above was concentrated, diluted with water, slowly adjusted to pH 7 with HCl (1 M) at a low temperature, extracted with EA (100 mL×4), concentrated, and purified by reversed-phase chromatography (ACN/water (0.02% TEA): 5%-30%) to give compound 3 (960 mg, yield: 95%) as a white solid.

LCMS: (M−H)⁻=945.6, Rt=7.68 min, purity: 99.9%

¹HNMR (400 MHz, MeOD) δ 8.86 (s, 1H), 7.46 (m, 2H), 7.41 (m, 4H), 7.30-7.25 (m, 6H), 7.17 (m, 1H), 6.83 (m, 4H), 5.36 (s, 1H), 4.54 (m, 3H), 4.35 (m, 1H), 4.13 (m, 1H), 3.90 (m, 1H), 3.76 (s, 6H), 3.16 (m, 5H), 3.04 (m, 2H), 2.54-2.43 (m, 8H), 2.23 (m, 3H), 1.64-1.55 (m, 4H), 1.43 (s, 2H), 1.29 (m, 8H), 1.02 (s, 9H)

### (5) Synthesis of Compound 3-CPG

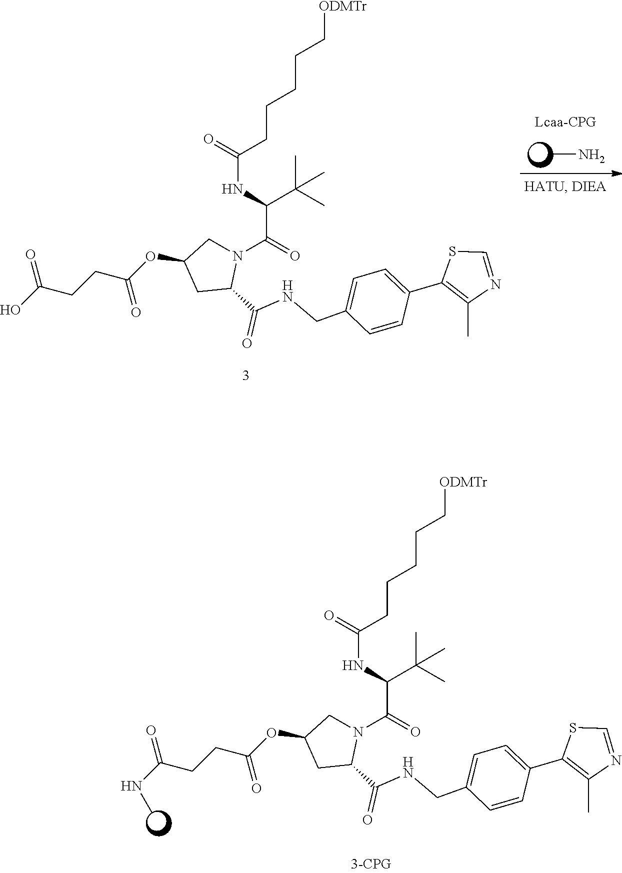

Compound 3 (200 mg, 0.211 mmol) was dissolved in acetonitrile (12 mL), and HATU (80 mg), DIEA (80 μL), and long chain alkylamine controlled pore glass (lcaa-CPG, pore size: 1000° A, 1000 mg) were separately added at room temperature. The mixture was shaken for 12 h. After the reaction was completed, the controlled pore glass was washed with acetonitrile, and CAP A (acetic anhydride:tetrahydrofuran=1:9, v/v, 4.0 mL) and CAP B (n-methylimidazole:pyridine:acetonitrile=15:10:75, v/v/v, 4.0 mL) were added. The mixture was shaken at room temperature for 1 h. The mixture described above was filtered and washed 3 times with acetonitrile (2 mL). After the mixture described above was lyophilized, compound 3-CPG (1000 mg) was obtained as a white powder.

### (6) Synthesis of AS1411-VH032

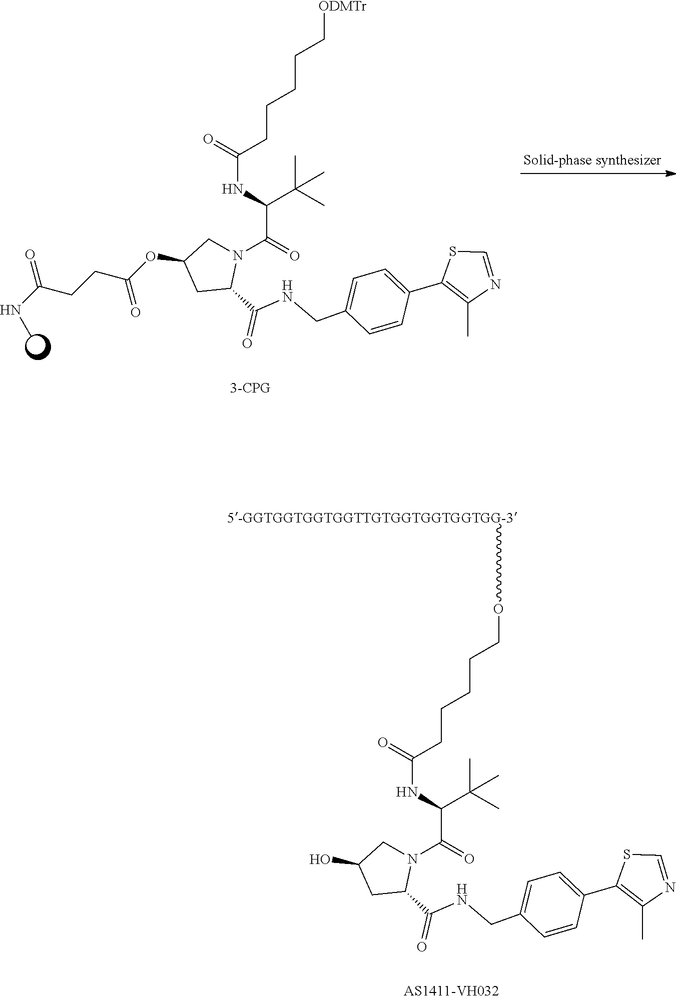

Compound 3-CPG was loaded into a synthesis column, and AS1411-VH032 was synthesized by a K&A H-8 solid-phase synthesizer (K&A, Germany) using the phosphoramidite method. Solid-phase synthesis using the phosphoramidite method is a method commonly used in the art and comprises 4 steps: deprotection, conjugation, capping, and oxidation. After the reaction was completed, 1.5 mL of an ammonia solution was added to CPG in each synthesis column, and the mixture was heated at 65° C. for 16 h. The supernatant was then collected and washed with water (1 mL×3). The crude product was purified by a protein purification system (Inscinstech, Unique AutoPreD) (column: NanoQ-15L, 4.7 mL; method: mobile phase A: a 40 mM aqueous NaOH solution, mobile phase B: an aqueous solution of 40 mM NaOH+2.0 M NaCl) to give the product AS1411-VH032 (white powder, 25.48 mg).

UPLC-MS (WATERS ACQUITY PREMIER): AS1411-VH032, m/z=8878.73832 [M]⁻ (deconvolution); Rt=10.911 min (260 nm). Mass error<50 ppm.

HPLC: AS1411-VH032, Rt=12.664 min (260 nm), purity: 99.337%.

The following was performed to further verify the ability of the novel PROTAC product prepared based on AS1411 for MDM2, i.e., AS1411-VH032, to degrade MDM2 at the protein level, as detailed below.

(1) A549 non-small cell lung cancer cells were incubated with 200 nM AS1411-VH032, and corresponding cell samples were collected at time gradients of 0 h, 3 h, 6 h, 9 h, 12 h, and 24 h for protein extraction and subsequent western blot assay to detect protein degradation. The results are shown in FIG. 53. The results indicate that AS1411-VH032 can significantly reduce the protein level of MDM2 in a time-dependent manner.

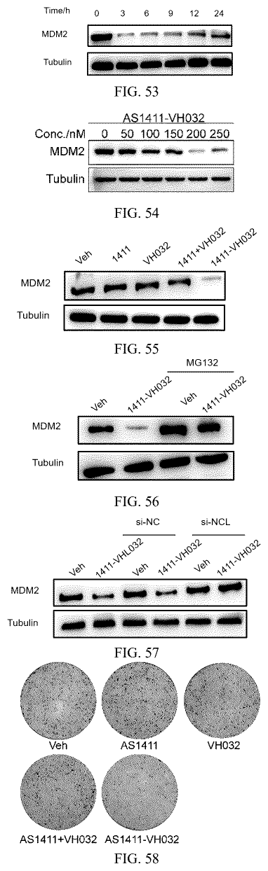

(2) A549 non-small cell lung cancer cells were incubated with AS1411-VH032 at concentrations of 0 nM, 50 nM, 100 nM, 150 nM, 200 nM, and 250 nM. After 3 h, cell samples were collected for protein extraction and subsequent western blot assay to detect protein degradation. The results are shown in FIG. 54. The results indicate that AS1411-VH032 can reduce the protein level of MDM2 in a concentration-dependent manner.

(3) A549 non-small cell lung cancer cells were incubated with 200 nM AS1411, VH032, AS1411-VH032, or AS1411+VH032. After 3 h, cell samples were collected for protein extraction and subsequent western blot assay to detect protein degradation. The results are shown in FIG. 55. The results indicate that only AS1411-VH032 can reduce the protein level of MDM2, while neither AS1411, VH032, nor AS1411+VH032 can reduce the protein level of MDM2.

(4) A549 non-small cell lung cancer cells were incubated with 200 nM AS1411-VH032 for 3 h, and proteasome inhibitor MG132 was added. Cell samples were collected for protein extraction and subsequent western blot assay.

The results are shown in FIG. 56. The results indicate that proteasome inhibitor MG132 can block the protein degradation of MDM2 by AS1411-VH032.

(5) A549 non-small cell lung cancer cells were transfected with si-NC or si-NCL and subsequently incubated with 200 nM AS1411-VH032 for 3 h. Cell samples were collected for protein extraction and subsequent western blot assay. The results are shown in FIG. 57. The results indicate that AS1411-VH032 cannot degrade MDM2 after NCL is silenced.

To verify the in vitro antitumor effect of AS1411-VH032, the following experiments were performed.

(6) A549 non-small cell lung cancer cells were seeded into a six-well plate for colony formation experiment. The medium containing 200 nM AS1411-VH032 was renewed every day during the experiment. The number of formed colonies was observed after 8 days, and the results are shown in FIG. 58. The results indicate that AS1411-VH032 can inhibit the colony formation of A549 cells.

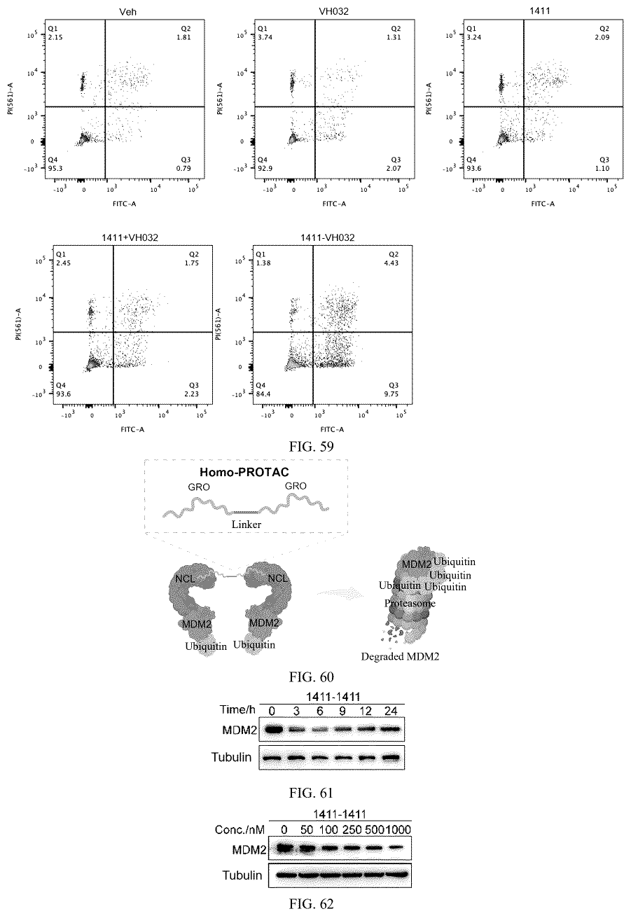

(7) A549 non-small cell lung cancer cells were seeded into a six-well plate for cell apoptosis experiment. The medium containing 200 nM AS1411-VH032 was renewed every day during the experiment. After 6 days, cell apoptosis analysis was performed using an Annexin V-FITC and PI double staining apoptosis detection kit, and the results are shown in FIG. 59. The results indicate that AS1411-VH032 can promote A549 cell apoptosis.

### Example 16: PROTAC Targeting MDM2

The objective of the present disclosure is to provide a homo-PROTAC degrader capable of degrading MDM2, and the homo-PROTAC degrader is formed by conjugating a first GRO to a second GRO via a linker. Therefore, the GROs serve not only as the recruitment element of a target protein in the homo-PROTAC but also as the recruitment element of an E3 ubiquitin ligase, and can simultaneously recruit MDM2 at both ends of the PROTAC molecule for “suicide” degradation, thereby achieving the treatment of MDM2-associated diseases, including tumors. The model of action of the homo-PROTAC degrader targeting MDM2 of the present disclosure is shown in FIG. 60.

The first GRO and the second GRO include AS1411 and a derivative or analog thereof (e.g., GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M). Further, in a preferred example of the present disclosure, both the first GRO and the second GRO are AS1411.

In a further preferred example of the present disclosure, the linker of the PROTAC includes a double-stranded DNA oligonucleotide with 3-30 A-T pairs, and more preferably, the linker of the PROTAC includes a double-stranded DNA oligonucleotide with 6 A-T pairs.

The preparation method for the AS1411-based homo-PROTAC degrader capable of degrading MDM2 was as follows:

The 3′ end of AS1411 was linked to the single strand with 6 A bases of the double-stranded DNA oligonucleotide by solid-phase synthesis to form a first conjugate product;
the 3′ end of AS1411 was linked to the single strand with 6 T bases of the double-stranded DNA oligonucleotide by solid-phase synthesis to form a second conjugate product;
the first conjugate product and the second conjugate product were separately heated in a metal bath at 95° C. for 5 min, cooled to 37° C., left to stand for 30 min, and mixed in a molar ratio of 1:1, so that the AS1411-based homo-PROTAC, i.e., AS1411-AS1411, was constructed by base complementary pairing of the double-stranded DNA oligonucleotide.

The following was performed to further verify the ability of the homo-PROTAC capable of degrading MDM2 prepared based on AS1411, i.e., AS1411-AS1411, to degrade MDM2 at the protein level, as detailed below.

(1) A549 non-small cell lung cancer cells were incubated with 1000 nM AS1411-AS1411, and corresponding cell samples were collected at time gradients of 0 h, 3 h, 6 h, 9 h, 12 h, and 24 h for protein extraction and subsequent western blot assay to detect protein degradation. The results are shown in FIG. 61. The results indicate that AS1411-AS1411 can significantly reduce the protein level of MDM2 in a time-dependent manner.

(2) A549 non-small cell lung cancer cells were incubated with AS1411-AS1411 at concentrations of 0 nM, 50 nM, 100 nM, 250 nM, 500 nM, and 1000 nM. After 12 h, cell samples were collected for protein extraction and subsequent western blot assay to detect protein degradation. The results are shown in FIG. 62. The results indicate that AS1411-AS1411 can reduce the protein level of MDM2 in a concentration-dependent manner.

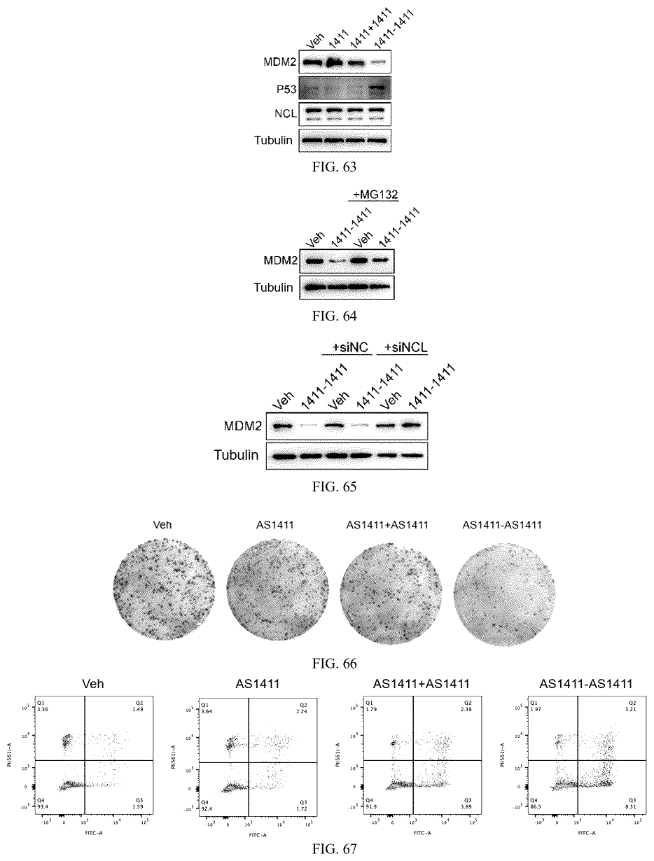

(3) A549 non-small cell lung cancer cells were incubated with 1000 nM AS1411, AS1411-AS1411, or AS1411+AS1411. After 12 h, cell samples were collected for protein extraction and subsequent western blot assay to detect protein degradation. The results are shown in FIG. 63. The results indicate that only AS1411-AS1411 can reduce the protein level of MDM2, while neither AS1411 nor AS1411+AS1411 can reduce the protein level of MDM2.

(4) A549 non-small cell lung cancer cells were incubated with 1000 nM AS1411-AS1411 for 12 h, and proteasome inhibitor MG132 was added. Cell samples were collected for protein extraction and subsequent western blot assay.

The results are shown in FIG. 64. The results indicate that proteasome inhibitor MG132 can block the protein degradation of MDM2 by AS1411-AS1411.

(5) A549 non-small cell lung cancer cells were transfected with si-NC or si-NCL and subsequently incubated with 1000 nM AS1411-AS1411 for 12 h. Cell samples were collected for protein extraction and subsequent western blot assay. The results are shown in FIG. 65. The results indicate that AS1411-AS1411 cannot degrade MDM2 after NCL is silenced.

To verify the in vitro antitumor effect of AS1411-AS1411, the following experiments were performed.

(6) A549 non-small cell lung cancer cells were seeded into a six-well plate for colony formation experiment. The medium containing 1000 nM AS1411-AS1411 was renewed every day during the experiment. The number of formed colonies was observed after 8 days, and the results are shown in FIG. 66. The results indicate that AS1411-AS1411 can inhibit the colony formation of A549 cells.

(7) A549 non-small cell lung cancer cells were seeded into a six-well plate for cell apoptosis experiment. The medium containing 1000 nM AS1411-AS1411 was renewed every day during the experiment. After 3 days, cell apoptosis analysis was performed using an Annexin V-FITC and PI double staining apoptosis detection kit, and the results are shown in FIG. 67. The results indicate that AS1411-AS1411 can promote A549 cell apoptosis.

In summary, ACCORDING TO THE AFOREMENTIONED DISCUSSION OF EXAMPLES 1 AND 2, IT IS VERIFIED THAT A GRO (E.G., AS1411) can capture NCL and MDM2, and the GRO (E.G., AS1411) can penetrate diseased cells (such as tumors) and exhibits significant colocalization with both NCL and MDM2. Therefore, as previously described, the GRO (E.G., AS1411) can recruit MDM2. While serving as a novel recruitment element of MDM2, a GRO (E.G., AS1411) endows PROTACs with targeting and penetration effects on diseased cells, reduces non-specific distribution of PROTACs in normal tissues, reduces toxic and side effects of PROTACs, and improves the ability and efficiency of PROTACs to enter diseased cells (such as tumors). PROTACs constructed based on GROs, a novel recruitment element of MDM2, can achieve the targeting effect on diseased cells (such as tumors) of PROTACs without additionally introducing other target heads that recognize diseased cells (such as tumors).

Compared with PROTACs that require additional introduction of other target heads that recognize diseased cells (such as tumors), the PROTACs are more ingenious and simple, which is conducive to synthesis and mass production.

As a novel recruitment element of MDM2, GROs can be used to construct macromolecular PROTACs, so as to avoid the problem that some difficult-to-drug target proteins do not have small molecule ligands and achieve cell penetration without any auxiliary means, thereby improving the application prospect of macromolecular PROTACs.

The PROTACs constructed in Examples 3-16 can all have a degrading effect on target proteins and are all suitable for the treatment of the various aforementioned diseases.

Although the present disclosure is illustrated by specific examples, it should be understood by those skilled in the art that various modifications and equivalent substitutions can be made to the present disclosure without departing from the scope of the present disclosure. In addition, various modifications can be made to the present disclosure for specific situations or materials without departing from the scope of the present disclosure. Therefore, the present disclosure is not limited to the specific examples disclosed, but should include all embodiments falling within the scope of the claims of the present disclosure.

The above description is only for the purpose of illustrating the preferred examples of the present disclosure, and is not intended to limit the scope of the present disclosure. Any modifications, equivalent substitutions, improvements, and the like made without departing from the spirit and principle of the present disclosure shall fall within the protection scope of the present disclosure.

1.-27. (canceled)

28. A targeted protein degrader, comprising a GRO, a linker, and a target protein ligand moiety, or comprising a GRO, a linker, and an E3 ubiquitin ligase ligand moiety, wherein the GRO is a guanine-rich oligonucleotide capable of specifically binding to NCL, and preferably, the GRO has stable G4 structural characteristics;
preferably, the targeted protein degrader has the following structure: 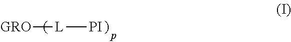
wherein
L is a linker;
PI is a target protein ligand moiety;
p is an integer of 1-100;
or
the targeted protein degrader has the following structure: 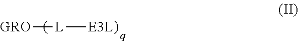
wherein
L is a linker;
E3L is an E3 ubiquitin ligase ligand moiety;
q is an integer of 1-100.

29. The targeted protein degrader according to claim 28, wherein the GRO is one of DNA aptamer AS1411 and an AS1411 derivative/analog; preferably, the AS1411 derivative/analog is one of GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M; preferably, the GRO is selected from:
AS1411, GRO29A, GRO15A, and AT11; more preferably, the GRO is AS1411;
preferably, the GRO comprises a chemical modification, a nucleic acid unit replacement, or a linkage to a functional group on the GRO, wherein
the chemical modification is that at least one base is modified, and the chemical modification comprises at least one of phosphorylation, methylation, amination, sulfydrylation, isotopication, phosphorothioate backbone modification, methoxy modification, and fluoro modification;
the nucleic acid unit replacement is that at least one nucleic acid unit is replaced by LNA, UNA, or GNA;
the functional group comprises at least one of a fluorophore, a radioactive group, a therapeutic drug, biotin, digoxigenin, a nano-luminescent material, a nucleic acid substance, or an enzyme label.

30. The targeted protein degrader according to claim 28, wherein L has the following structure: 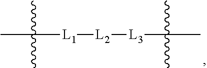
wherein
L₁ is a divalent group linked to the GRO and is selected from: a single bond, —O—(C₀-C₆ alkylene)-, —S—(C₀-C₆ alkylene)-, —N(R^{L1})—(C₀-C₆ alkylene)-, —N(R^{L2})C(O)—(C₀-C₆ alkylene)-, —OP(O)(OR^{L1})O—(C₀-C₆ alkylene)-, —C(O)—(C₀-C₆ alkylene)-, —C(S)—(C₀-C₆ alkylene)-, and —CON(R^{L1})—(C₀-C₆ alkylene)-;
L₃ is a divalent group linked to PI or E3L and is selected from: a single bond, —O—(C₀-C₆ alkylene)-, —S—(C₀-C₆ alkylene)-, —C(O)—(C₀-C₆ alkylene)-, —C(S)—(C₀-C₆ alkylene)-, —N(R^{L3})—(C₀-C₆ alkylene)-, —CON(R^{L3})—(C₀-C₆ alkylene)-, —N(R^{L3})CO—(C₀-C₆ alkylene)-, —SO₂—(C₀-C₆ alkylene)-, and —SO—(C₀-C₆ alkylene)-;
L₂ is a single bond or a divalent, saturated or unsaturated, linear or branched C1-C50 hydrocarbon chain, wherein 0-6 methylene units in the hydrocarbon chain are independently substituted with: —CY—, —O—, —S—, —S—S—, —C(O)—, —C(S)—, —C(O)O—, —OC(O)—, —OC(O)O—, —C(O)N(R^{L2})—, —N(R^{L2})C(O)—, —N(R^{L2})C(O)O—, —N(R^{L2})C(O)N(R^{L2})—, —N(R^{L2})—, —S(O)₂—, —S(O)₂N(R^{L2})—, —N(R^{L2})S(O)₂—, —S(O)—, —S(O)N(R^{L2})—, —N(R^{L2})S(O)—, —P(O)(OR^{L2})O—, —P(O)—, —P(O)N(R^{L2})—, —P(O)(N(R^{L2})₂)—, —OP(O)(OR^{L2})₂N(R^{L2})—, —P(O)(OR^{L2})₂N(R^{L2})—, —N(R^{L2})P(O)(OR^{L2})O—, —N(R^{L2})P(O)—, —Si(R^{L2})₂—, —C(═N—CN)—, 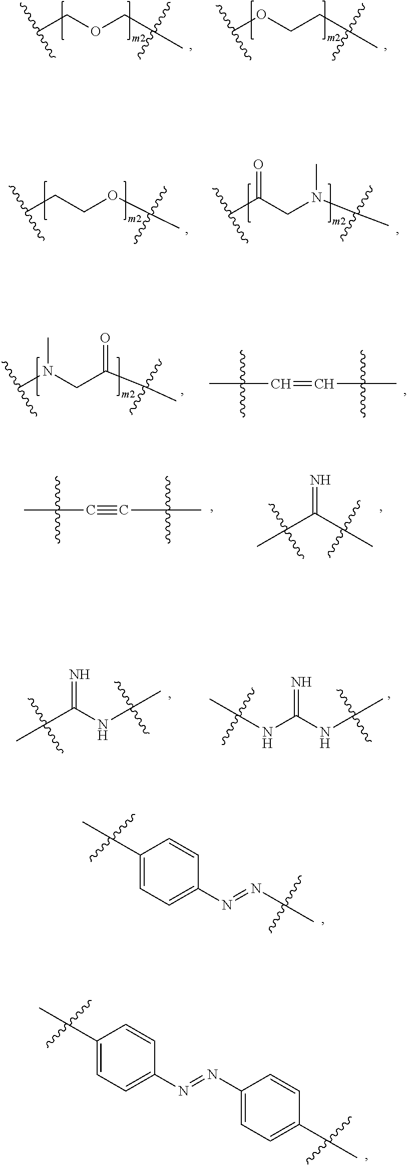
an amino acid residue, a nucleotide residue, an oligonucleotide residue, or an oligopeptide residue, wherein m2 is selected from integers between 1 and 10, and each —CY— is independently an optionally substituted divalent ring selected from: arylene, cycloalkylene, and heterocyclylene; H in the hydrocarbon chain may be optionally substituted with one or more groups selected from: halogen, cyano, nitro, azido, —OR^{L0}, —C(O)R^{L0}, —C(S)R^{L}o, —C(O)OR^{L0}, —C(S)SR^{L0}, —OC(O)R^{L0}, —OC(S)R^{L0}, —OC(S)SR^{L0}, —C(O)N(R^{L0})₂, —OC(O)N(R^{L0})₂, —N(R^{L0})C(O)OR^{L0}, —N(R^{L0})SO₂R^{L0}, —SO₂N(R^{L0})₂, —OSO₂N(R^{L0})₂, —N(R^{L0})C(O)R^{L0}, —N(R^{L0})₂, —SR^{L0}, —SOR^{L0}, —SO₂R^{L0}, —OSO₂R^{L0}, C₁-C₁₀ alkyl, C₂-C₁₀ alkenyl, C₂-C₁₀ alkynyl, C₁-C₁₀ haloalkyl, C₁-C₁₀ haloalkoxy, —(C₀-C₆ alkylene)-(C₃-C₁₀ cycloalkyl), —(C₀-C₆ alkylene)-(C₆-C₁₀ aryl), and —(C₀-C₆ alkylene)-(4- to 10-membered heterocyclyl);
R^{L0}, R^{L1}, R^{L2}, and R^{L3} are independently selected from: H, C₁-C₁₀ alkyl, C₂-C₁₀ alkenyl, C₂-C₁₀ alkynyl, —(C₀-C₆ alkylene)-(C₃-C₁₀ cycloalkyl), —(C₀-C₆ alkylene)-(C₆-C₁₀ aryl), and —(C₀-C₆ alkylene)-(4- to 10-membered heterocyclyl), wherein H in the C₁-C₁₀ alkyl, C₂-C₁₀ alkenyl, C₂-C₁₀ alkynyl, C₀-C₆ alkylene, C₃-C₁₀ cycloalkyl, C₆-C₁₀ aryl, or 4- to 10-membered heterocyclyl is optionally substituted with one or more groups selected from: halogen, cyano, nitro, azido, hydroxy, amino, sulfydryl, carboxyl, C₁-C₁₀ alkyl, C₂-C₁₀ alkenyl, C₂-C₁₀ alkynyl, C₁-C₁₀ haloalkyl, C₁-C₁₀ haloalkoxy, —(C₀-C₆ alkylene)-(C₃-C₁₀ cycloalkyl), —(C₀-C₆ alkylene)-(C₆-C₁₀ aryl), and —(C₀-C₆ alkylene)-(4- to 10-membered heterocyclyl);
preferably, L is linked to any base, sugar, or phosphate backbone at the 3′ end or 5′ end or in the middle of the GRO.

31. The targeted protein degrader according to claim 28, wherein L₂ is an oligonucleotide residue, such as a DNA oligonucleotide residue, an RNA oligonucleotide residue, or a DNA/RNA hybrid oligonucleotide residue;
preferably, the oligonucleotide consists of A and/or T;
more preferably, L₂ is a single-stranded oligonucleotide residue comprising 3-30 A or T;
more preferably, L₂ is a double-stranded oligonucleotide residue comprising 3-30 A-T pairs.

32. The targeted protein degrader according to claim 28, wherein L₂ is C₁-C₂₀ linear alkylene, wherein 0-6 methylene units in the alkylene are independently substituted with the following groups: —O—, —C(O)—, —C(O)O—, —OC(O)—, —N(R^{L2})—, —C(O)N(R^{L2})—, —N(R^{L2})C(O)—, 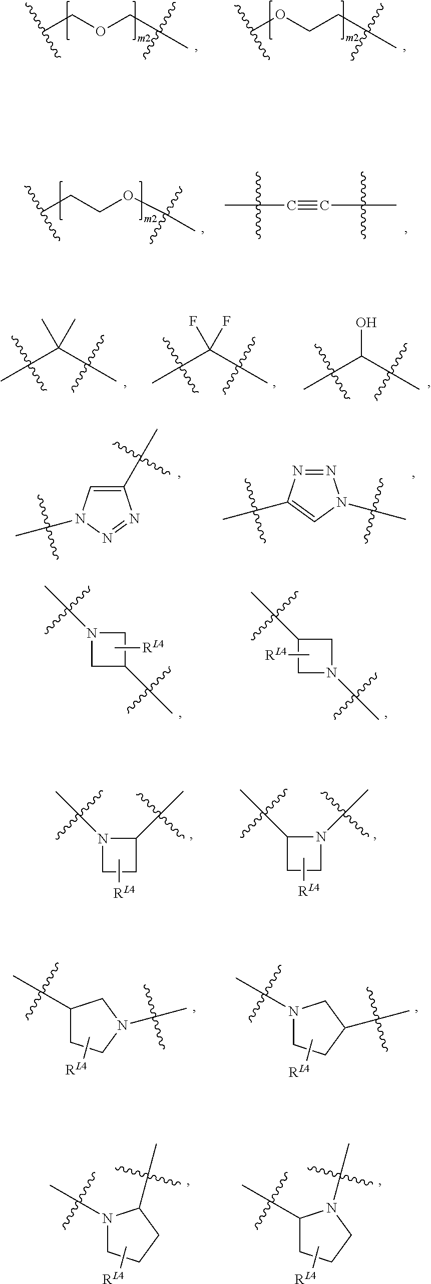 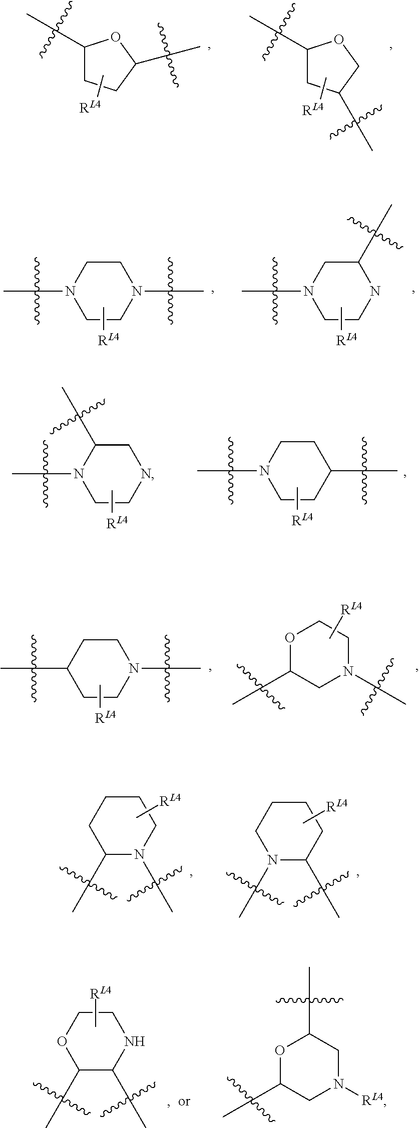
wherein, each R^{L2} is independently selected from: H and C₁-C₆ alkyl, and each R^{L4} is independently selected from: OH and C₁-C₆ alkoxy;
preferably, L₂ is selected from: C₁-C₂₀ linear alkylene, —(CH₂CH₂O)ₘ₂—CH₂—, —(CH₂CH₂O)ₘ₂—CH₂CH₂—, —CH₂—(CH₂CH₂O)ₘ₂—CH₂—, —CH₂CH₂—(CH₂CH₂O)ₘ₂—CH₂—, —CH₂CH₂—(CH₂CH₂O)ₘ₂—CH₂CH₂—, —(C₁-C₁₀ alkylene)-O—(C₁-C₁₀ alkylene)-, —(C₁-C₁₀ alkylene)-NH—(C₁-C₁₀ alkylene)-, —(C₁-C₁₀ alkylene)-C(O)NH—(C₁-C₁₀ alkylene)-, —(C₁-C₁₀ alkylene)-NHC(O)—(C₁-C₁₀ alkylene)-, —(C₁-C₆ alkylene)-O—(C₁-C₆ alkylene)-C(O)NH—(C₁-C₆ alkylene)-, —(C₁-C₆ alkylene)-O—(C₁-C₆ alkylene)-NHC(O)—(C₁-C₆ alkylene)-, 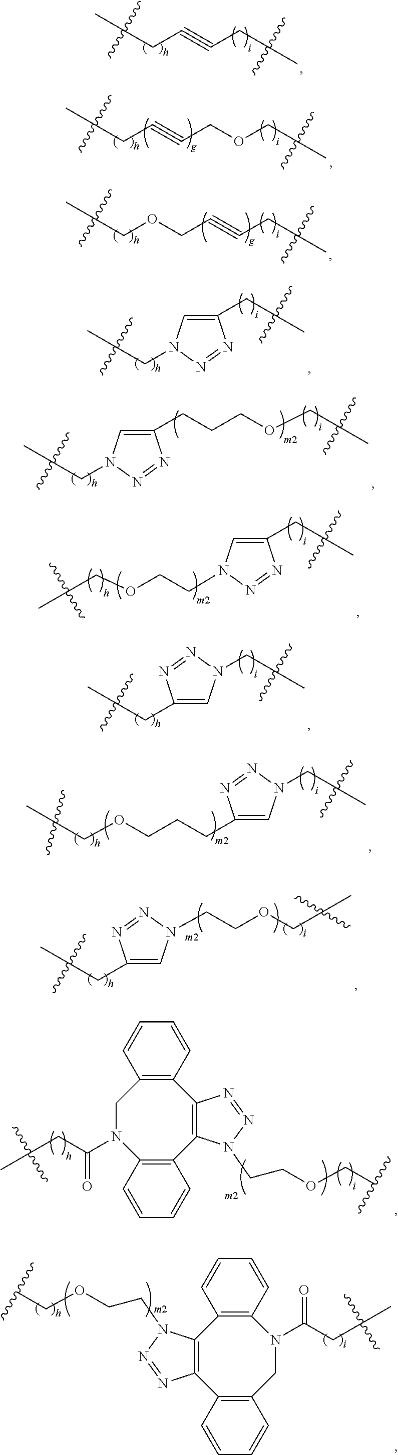 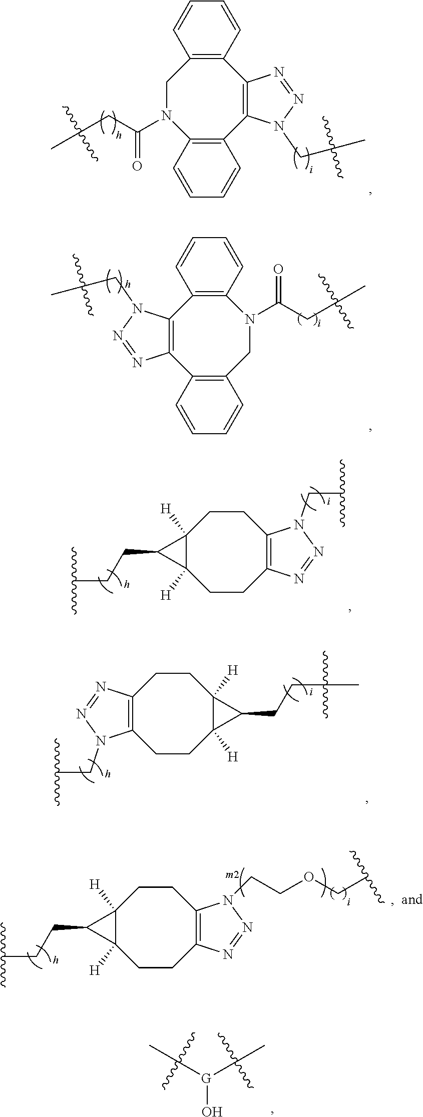
wherein m2 is selected from integers between 1 and 10, g is 0 or 1, h is selected from integers between 0 and 10, i is selected from integers between 0 and 10, and G is any suitable trivalent group;
more preferably, L₂ is selected from:

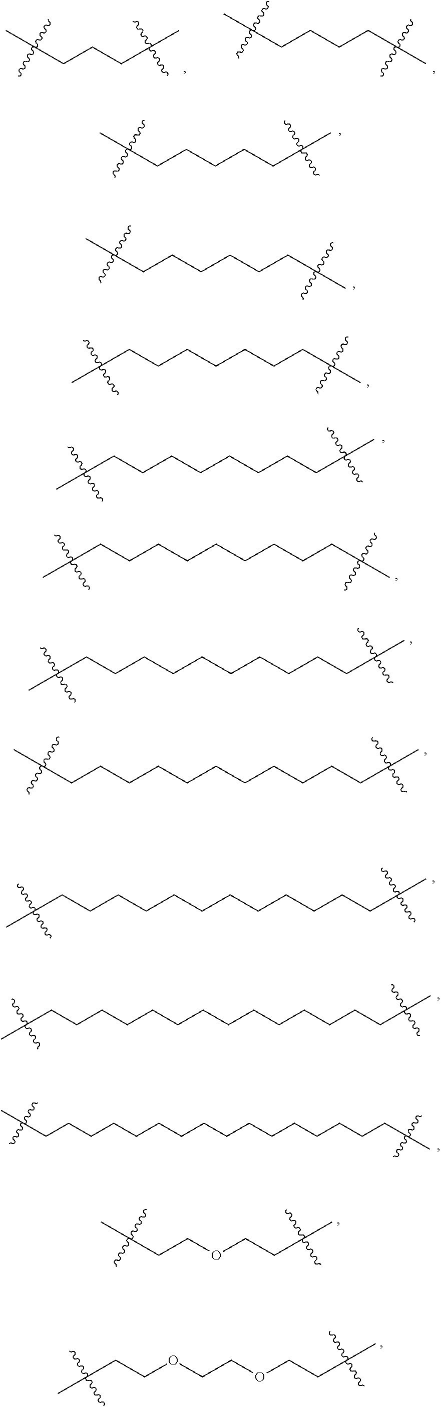

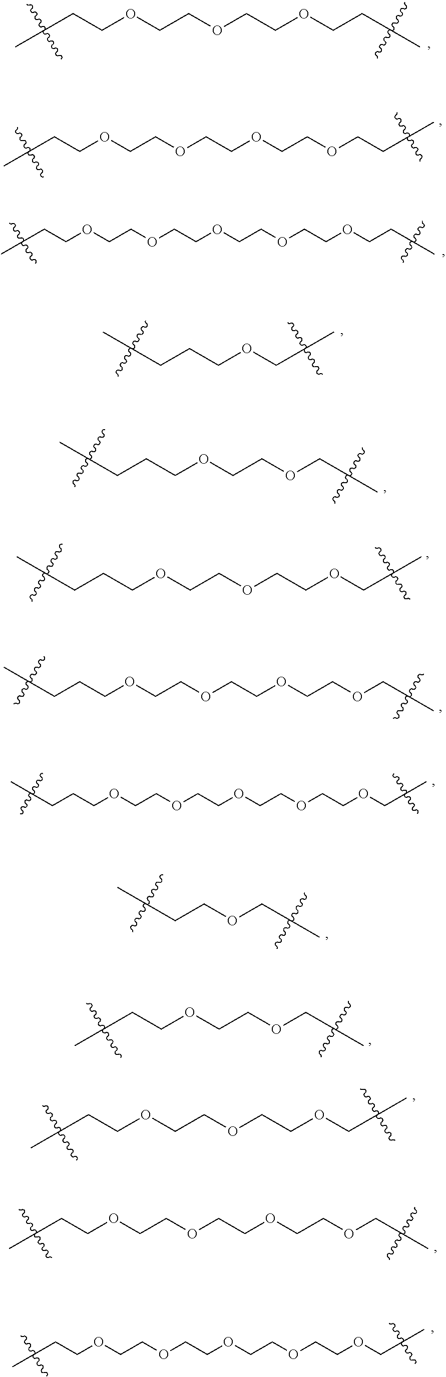

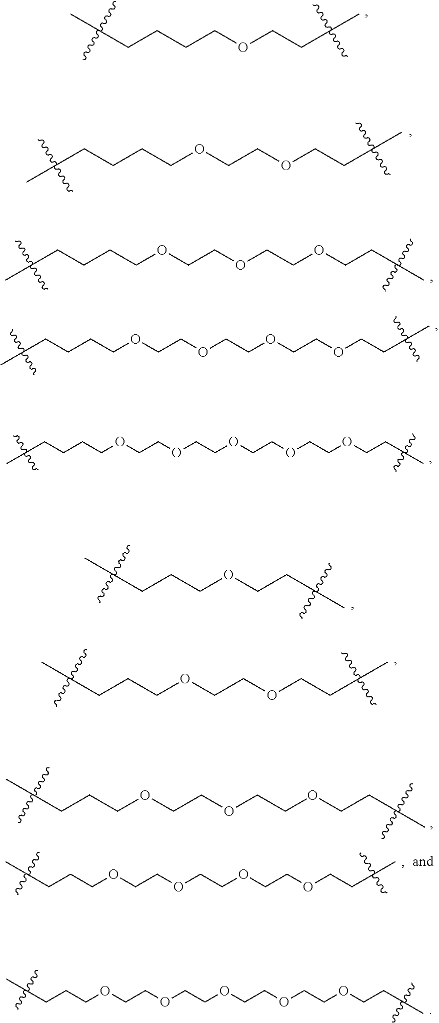

33. The targeted protein degrader according to claim 28, wherein L₂ is selected from C1-C20 linear alkylene, —(C₀-C₆ alkylene)-(CH₂CH₂O)ₘ₂-(1C₁-C₆ alkylene)-, -(C₁-C₁₀ alkylene)-O—(C₁-C₁₀ alkylene)-, —(C₁-C₁₀ alkylene)-NH—(1C₁-C₁₀ alkylene)-, —(C₁-C₁₀ alkylene)-C(O)NH—(C₁-C₁₀ alkylene)-, —(C₁-C₁₀ alkylene)-NHC(O)—(C₁-C₁₀ alkylene)-, —(C₁-C₆ alkylene)-O—(C₁-C₆ alkylene)-C(O)NH—(C₁-C₆ alkylene)-, and —(C₁-C₆ alkylene)-O—(C₁-C₆ alkylene)-NHC(O)—(C₁-C₆ alkylene)-, wherein 1 methylene unit in the alkylene is substituted with 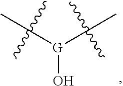
and G is any suitable trivalent group;
preferably, L₂ is 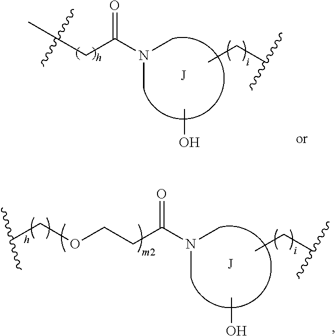
ring J is a saturated 4- to 6-membered heterocyclic ring, m2 is selected from integers between 1 and 10, h is selected from integers between 0 and 10, and i is selected from integers between 0 and 10;
more preferably, L₂ is 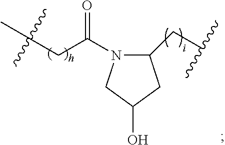
more preferably, L₂ is selected from:

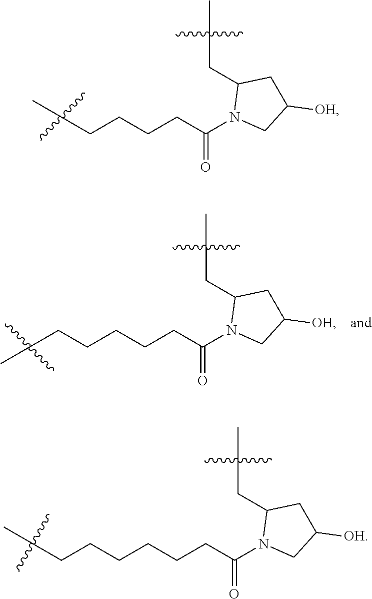 [SMILES: *CCCCC(=O)N1CC(O)CC1C* ; *CCCCCC(=O)N1CC(O)CC1C* ; *CCCCCCC(=O)N1CC(O)CC1C*]

34. The targeted protein degrader according to claim 28, wherein L₂ is C1-C20 linear alkylene, wherein 1-3 methylene units are independently substituted with the following groups: —CY—, 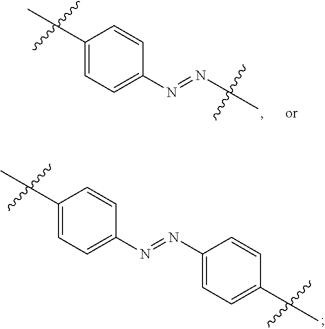 [SMILES: */N=N/c1ccc(*)cc1 ; *c1ccc(/N=N/c2ccc(*)cc2)cc1]
optionally, L₂ further comprises a group selected from: —O—, —C(O)—, —N(R^{L2})—, —C(O)N(R^{L2})—, —N(R^{L2})C(O)—, 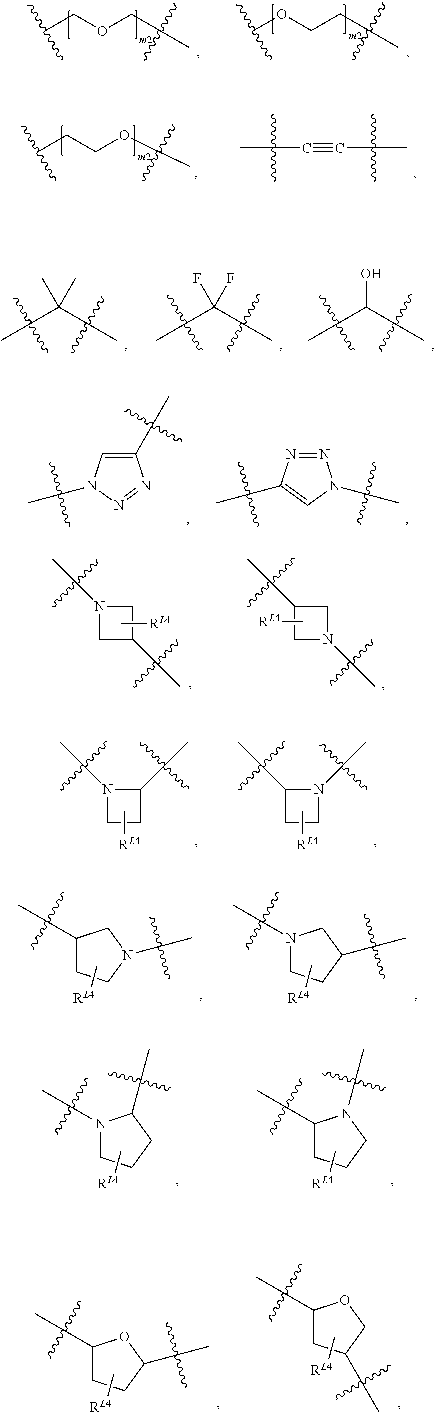 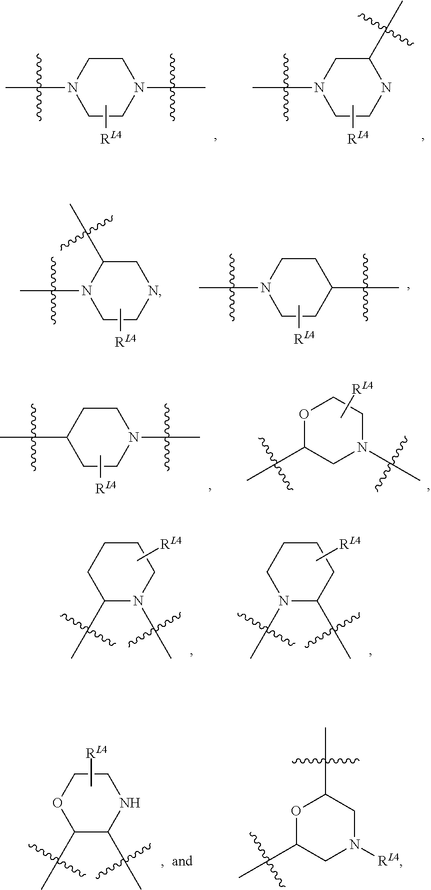
wherein each R^{L2} is independently selected from: H and C₁-C₆ alkyl, and each R^{L4} is independently selected from: OH and C₁-C₆ alkoxy;
preferably, the —CY— is selected from: 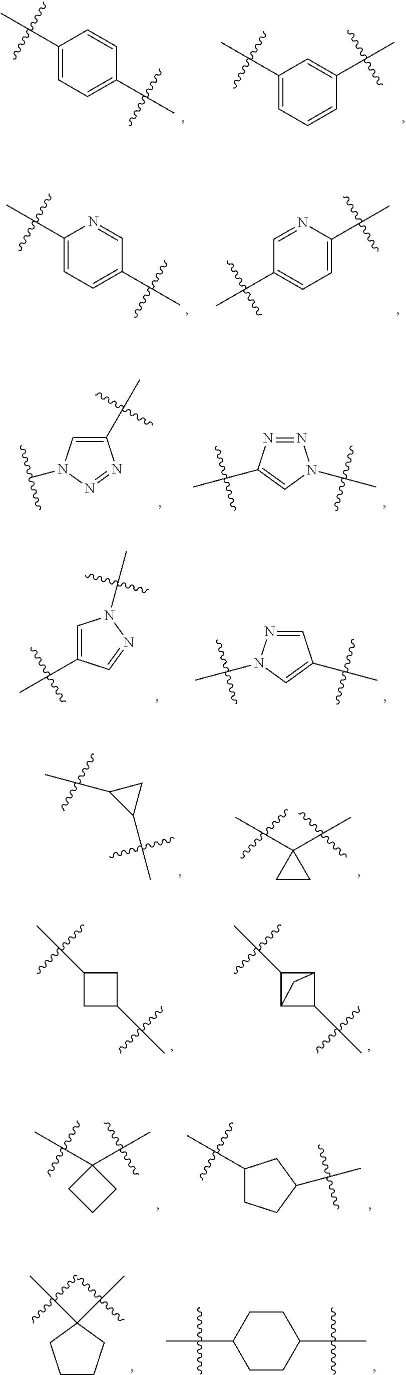 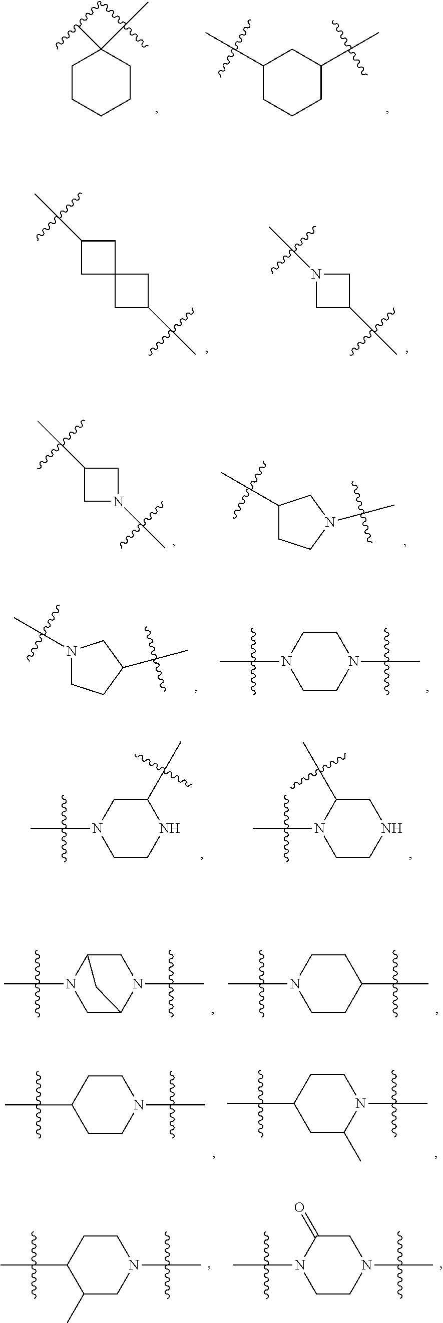 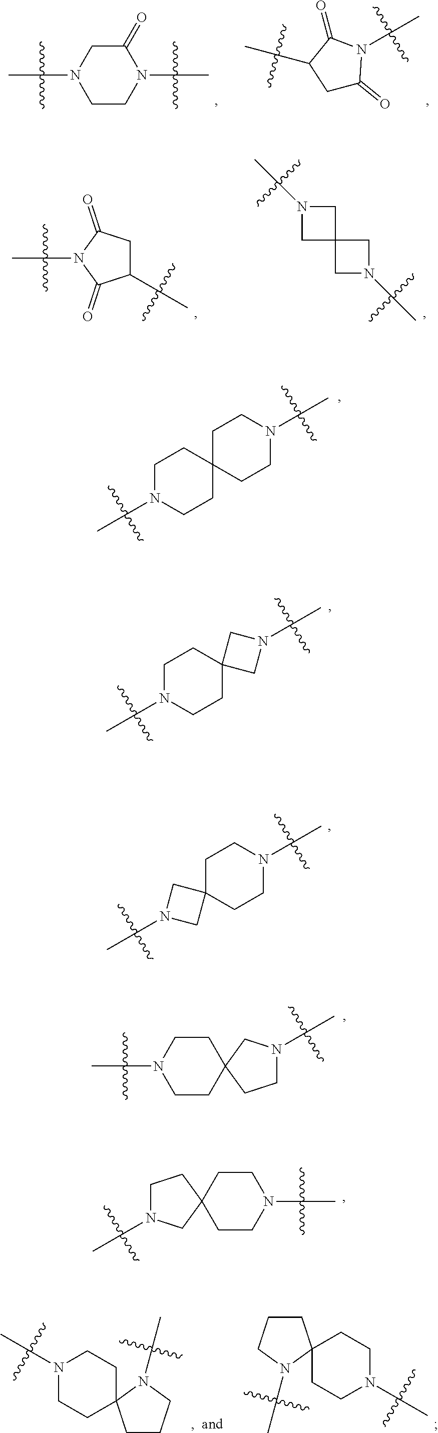
more preferably, L₂ is selected from:

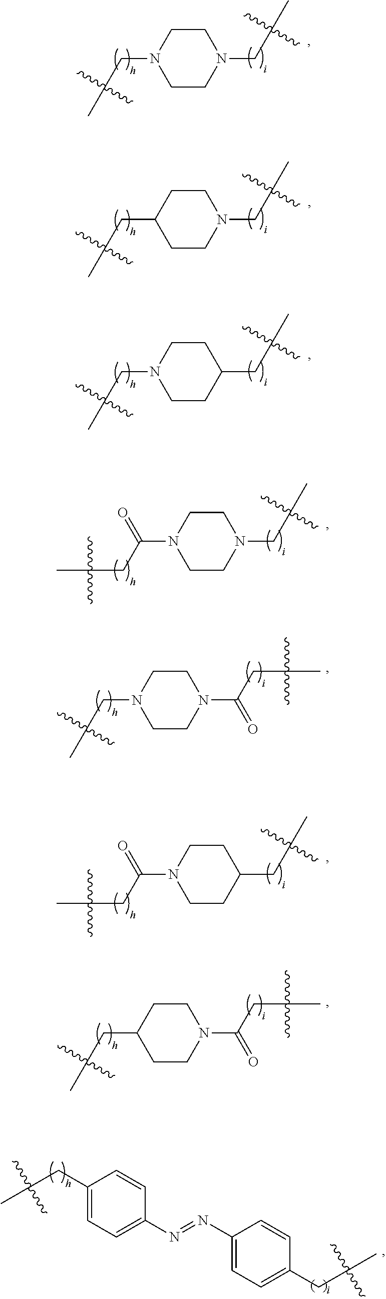

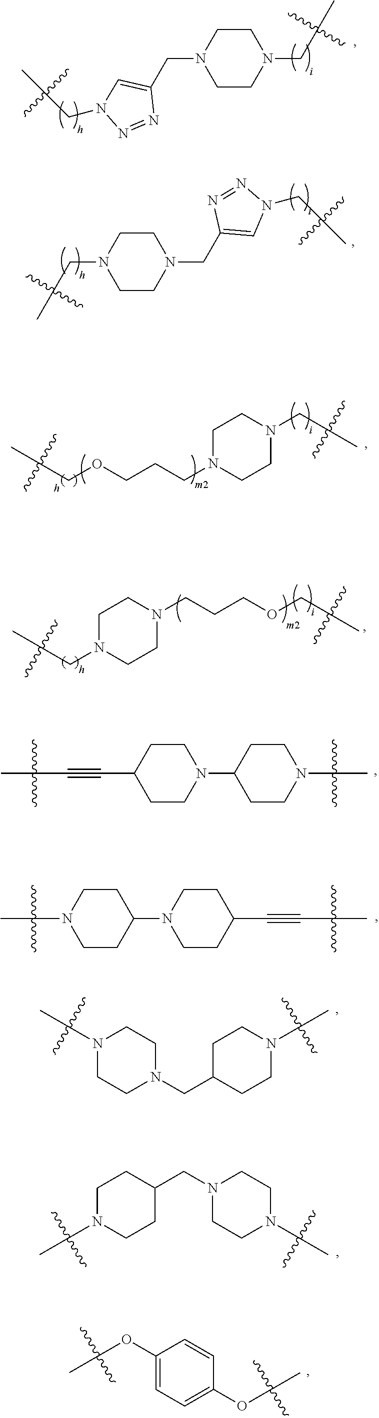

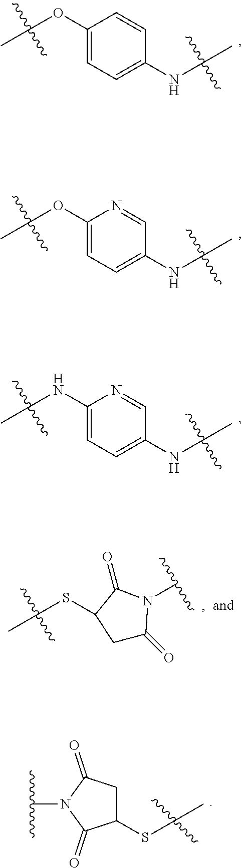 [SMILES: *Nc1ccc(O*)cc1 ; *Nc1ccc(O*)nc1 ; *Nc1ccc(N*)nc1 ; *SC1CC(=O)N(*)C1=O ; *SC1CC(=O)N(*)C1=O]

35. The targeted protein degrader according to claim 28, wherein the target protein ligand is a small molecule compound, an oligonucleotide, an oligopeptide, a polypeptide, or a protein;
preferably, the target protein ligand is an oligonucleotide, such as a DNA oligonucleotide, an RNA oligonucleotide, or a DNA/RNA hybrid oligonucleotide;
more preferably, L is linked to any base, sugar, or phosphate backbone at the 3′ end or 5′ end or in the middle of an oligonucleotide strand of the target protein ligand.

36. The targeted protein degrader according to claim 35, wherein the target protein is selected from:

| | | | | |
|---|---|---|---|---|
| FAK | USP7 | GPX4 | PI3K | JAK |
| MetAP-2 | ER | AR | BET | IKAROS |
| RTK | ALC | EGFR | HER2 | TRK |
| BTK | STAT | IRAK4 | BCL-XL | TBK1 |
| FRS2 | STAT3 | HDAC6 | PDEδ | MAFF |
| Tau | GSK-3 | α-syn | mHtt | TDP-43 |
| FUS | SOD | NS3/4A | RIPK2 | BCR-ABL |
| PCAF/GCN5 | SHP2 | Bcl-2 | PD-L1 | NAMPT |
| EGFR-L858R | NA | STING | FKBP12 | TK |
| BRDT | FAP | Hsp70 | NLRP3 | cGAS |
| FLT3-ITD | c-MET | ALK | Akt | CK2 |
| FLT3 | Sirt2 | Pirin | SMAD3 | ARNT |
| CDK2 | CDK4 | CDK6 | CDK9 | ERK1 |
| BRD2 | BRD4 | BRD6 | BRD9 | ERK2 |
| IKZF1 | IKZF3 | RAR | Bcr/Abl | c-Abl |
| DAPK1 | PSD-95 | TRIM24 | CRABP-I | CRABP-II |
| TACC3 | AHR | FKBP12 | ERRα | X protein |
| Huntingtin | DHODH | Halo Tags | NQO1 | GSTP1 |
| CDK12 | PARP1 | p53 | HMGCR | LXRs |
| VEGFR-2 | SMARCA4 | EED | SMARCA2 | SGK3 |
| EGFR/PARP | ERK5 | KRAS | EZH2 | MEK |
| ER/GPER | XPO1 | Myc | VEGF | |

preferably, the target protein ligand is a c-MET ligand, such as DNA aptamer SL1 having the nucleotide sequence set forth in SEQ ID NO: 19; or
preferably, the target protein ligand is a STAT3 protein ligand, such as double-stranded transcription factor decoy oligonucleotide S3 that specifically recognizes STAT3, wherein the sense strand S3-F of S3 is set forth in SEQ ID NO: 5, and the antisense strand S3-R of S3 is set forth in SEQ ID NO: 6; or
preferably, the target protein ligand is a ligand of c-Myc, such as a double-stranded transcription factor decoy oligonucleotide that specifically recognizes c-Myc, wherein the sense strand sequence MYC-F of the oligonucleotide is set forth in SEQ ID NO: 7, and the antisense strand sequence MYC-R of the oligonucleotide is set forth in SEQ ID NO: 8; or
preferably, the target protein ligand is a ligand of p53-R175H, such as RNA aptamer R175Hapt having the sequence set forth in SEQ ID NO: 9; or
preferably, the target protein ligand is a ligand of AR-V7, such as RNA oligonucleotide lncRV having the sequence set forth in SEQ ID NO: 10 or lncV7 having the sequence set forth in SEQ ID NO: 11; or
preferably, the target protein ligand is a ligand of VEGF165, such as single-stranded DNA aptamer V7t1 having the sequence set forth in SEQ ID NO: 12; or
preferably, the target protein ligand is a ligand of MDM2, such as a GRO, more preferably one of DNA aptamer AS1411 and an AS1411 derivative/analog; preferably, the AS1411 derivative/analog is one of GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M.

37. The targeted protein degrader according to claim 36, wherein L₂ is an oligonucleotide residue, and L₁ and/or L₃ are/is a single bond or O.

38. The targeted protein degrader according to claim 28, wherein the target protein ligand is a c-MET ligand;
preferably, the targeted protein degrader represented by general formula I has the following structure: 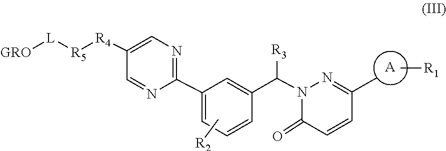
wherein
ring A is an aromatic ring or a heterocyclic ring;
R₁ is one or more independent substituents on ring A and is selected from: halogen, —CN, —NO₂, —OCF₃, C₀₋₁₀ alkyl, C₁₋₁₀ haloalkyl, —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —OC₀₋₁₀ alkyl, —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, and 4- to 10-membered heterocyclyl;
R₂ is one or more independent substituents on a benzene ring and is selected from: halogen, —CN, —NO₂, —CF₃, —OCF₃, C₀₋₁₀ alkyl, C₁₋₁₀ haloalkyl, —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —OC₀₋₁₀ alkyl, —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, and 4- to 10-membered heterocyclyl;
R₃ is selected from: C₀₋₁₀ alkyl and C₁₋₁₀ haloalkyl;
R₄ is —(C₀₋₆ alkyl)-R₄′—(C₀₋₆ alkyl)-, wherein R₄′ is selected from: a single bond, O, S, N(C₀₋₁₀ alkyl), C(O), OC(O), C(O)O, N(C₀₋₁₀ alkyl)C(O), C(O)N(C₀₋₁₀ alkyl), SO₂, SO₂N(C₀₋₁₀ alkyl), 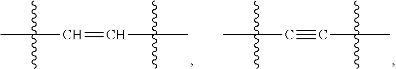
C₃₋₆ cycloalkylene, and 4- to 10-membered heterocyclylene;
R₅ is selected from: a single bond, O, S, N(C₀₋₁₀ alkyl), C(O), OC(O), C(O)O, N(C₀₋₁₀ alkyl)C(O), C(O)N(C₀₋₁₀ alkyl), SO₂, SO₂N(C₀₋₁₀ alkyl), C₃₋₆ cycloalkylene, and 4- to 10-membered heterocyclylene.

39. The targeted protein degrader according to claim 38, wherein moiety 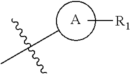
is selected from the following structures: 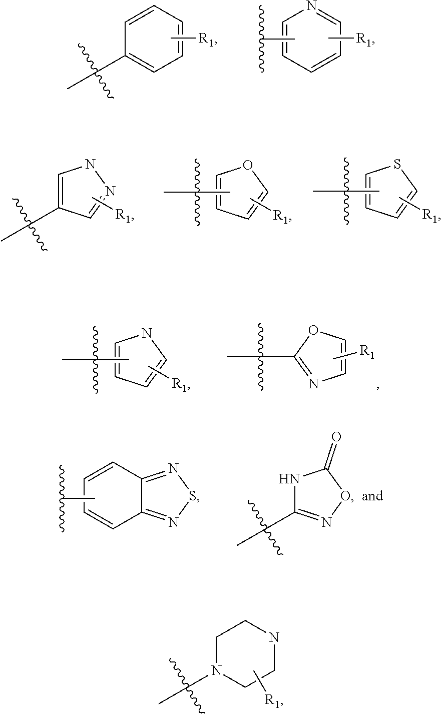
and preferably, moiety 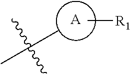
is 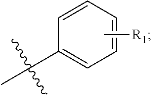
preferably, R₁ is selected from: H, halogen (e.g., F and Cl), —CN, —NO₂, —OCF₃, C₁₋₃ alkyl, C₁₋₃ haloalkyl, —OC₀₋₃ alkyl, and —N(H)CO(C₁₋₃ alkyl).

40. The targeted protein degrader according to claim 38, wherein R₄′ is selected from: a single bond, O, S, N(H), C(O), N(H)C(O), C(O)N(H), 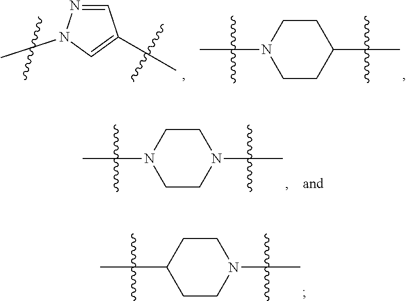 [SMILES: *c1cnn(*)c1 ; *C1CCN(*)CC1 ; *N1CCN(*)CC1 ; *C1CCN(*)CC1]
preferably, R₄ is —(C₁₋₆ alkyl)-O—.

41. The targeted protein degrader according to claim 38, wherein R₅ is selected from: a single bond, O, S, N(C₀₋₃ alkyl), N(H)C(O), C(O)N(H), 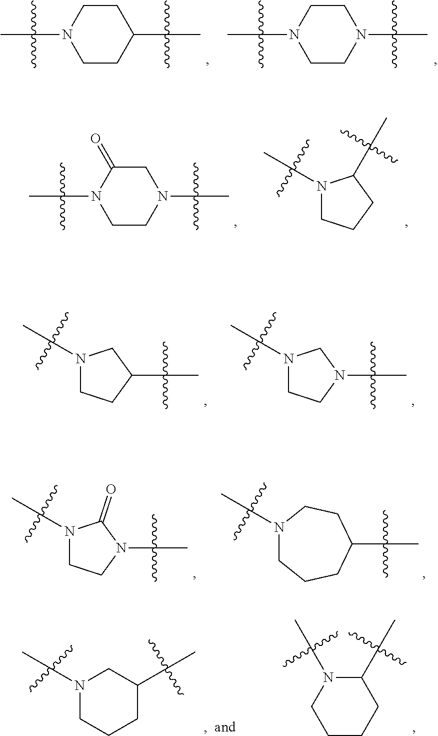
and preferably, R₅ is

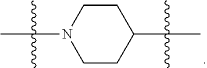 [SMILES: *C1CCN(*)CC1]

42. The targeted protein degrader according to claim 28, wherein the targeted protein degrader represented by general formula I has the following structure: 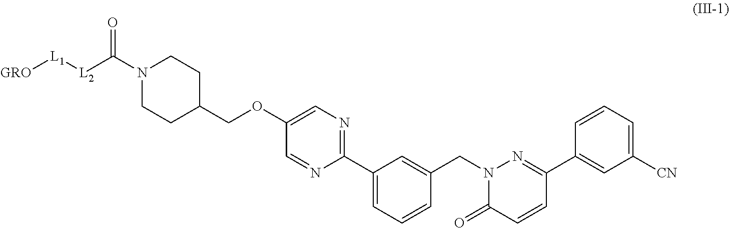
preferably, L₂ is 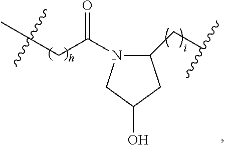
h is selected from integers between 0 and 10, and i is selected from integers between 0 and 10;
preferably, L₁ is a single bond or O.

43. The targeted protein degrader according to claim 28, wherein the targeted protein degrader has the following structure:

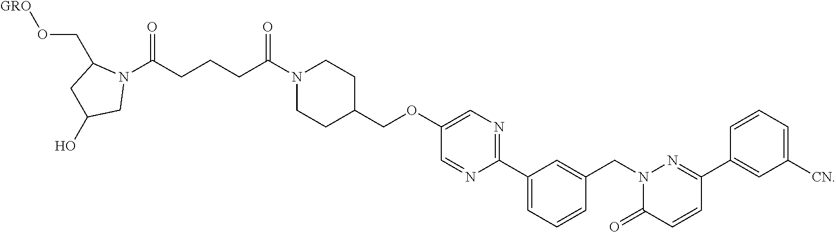

44. The targeted protein degrader according to claim 28, wherein in general formula II, the E3 ligase ligand is selected from: a CRBN protein ligand, a VHL protein ligand, an MDM2 protein ligand, an IAP protein ligand, a cbl-b protein ligand, a DCAF15 protein ligand, a DCAF16 protein ligand, a KEAP1 protein ligand, an RNF4 protein ligand, an RNF114 protein ligand, a β-TrCP protein ligand, and an FEM1B protein ligand;
preferably, the CRBN protein ligand comprises: an amide compound, a phthalimide compound, thalidomide or a derivative thereof, lenalidomide or a derivative thereof, and pomalidomide or a derivative thereof;
preferably, the VHL protein ligand is VH032 or a derivative thereof;
preferably, the MDM2 protein ligand is Nutlin-3a or a derivative thereof or the GRO;
preferably, the IAP protein ligand is selected from: Bestatin, LCL-161, MV-1, and a derivative thereof.

45. The targeted protein degrader according to claim 28, wherein in general formula II, the E3L moiety has the following structures: 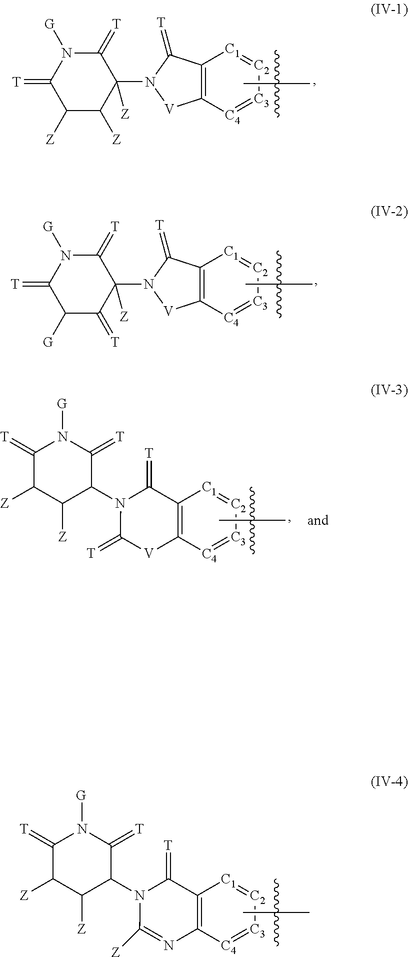
wherein C₁, C₂, C₃, and C₄ are independently selected from: CR¹⁰¹ and N, wherein each R¹⁰¹ is selected from: —OH, halogen, —CN, —NO₂, —CF₃, —OCF₃, C₀₋₁₀ alkyl, —N(C₀₋₁₀ alkyl)(C₀₋₁₀ alkyl), —N(C₀₋₁₀ alkyl)CO(C₀₋₁₀ alkyl), —N(C₀₋₁₀ alkyl)CON(C₀₋₁₀ alkyl), —N(C₀₋₁₀ alkyl)SO₂(C₀₋₁₀ alkyl), —OC₀₋₁₀ alkyl, —SC₀₋₁₀ alkyl, —SO(C₀₋₁₀ alkyl), —SO₂(C₀₋₁₀ alkyl), —SO₂N(C₀₋₁₀ alkyl)(C₀₋₁₀ alkyl), —COO(C₀₋₁₀ alkyl), —OCO(C₀₋₁₀ alkyl), —CON(C₀₋₁₀ alkyl)(C₀₋₁₀ alkyl), —CO(C₀₋₁₀ alkyl), C₃₋₆ cycloalkyl, —O heterocycloalkyl, —N heterocycloalkyl, —S heterocycloalkyl, —N heterocycloaryl, —O heterocycloaryl, and —S heterocycloaryl;
T is selected from: O and S;
V is selected from: O, S, 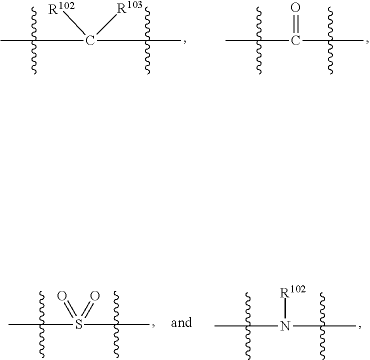
wherein R¹⁰² and R¹⁰³ are independently selected from: C₀₋₁₀ alkyl, cycloalkyl, and heterocycloalkyl;
G and Z are independently selected from: —OH, C₀₋₁₀ alkyl, C₃₋₁₀ cycloalkyl, O-containing heterocycloalkyl, N-containing heterocycloalkyl, and S-containing heterocycloalkyl;
preferably, the E3L moiety has the following structure: 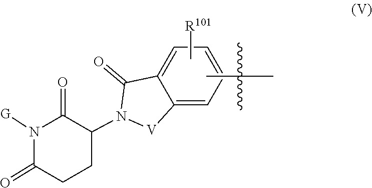
more preferably, the E3L moiety has the following structures:

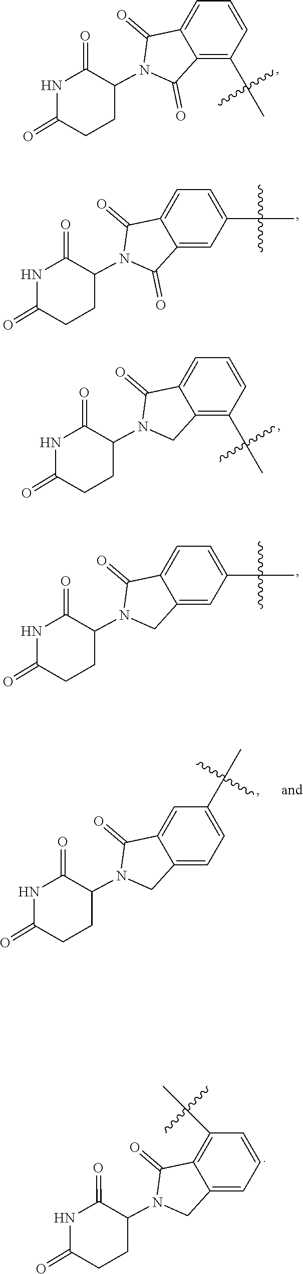 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1CN(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2 ; *c1cccc2c1C(=O)N(C1CCC(=O)NC1=O)C2]

46. The targeted protein degrader according to claim 28, wherein E3L has the following structure: 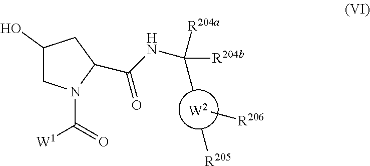
wherein W¹ is selected from aryl, heteroaryl, and 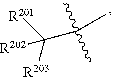
wherein R²⁰¹ and R²⁰² are independently selected from: H, alkyl, cycloalkyl, hydroxyalkyl, haloalkyl, and heteroaryl; or
R²⁰¹ and R²⁰², together with the carbon atom to which they are attached, form cycloalkyl; R²⁰³ is selected from: alkyl, alkoxy, aryl, heterocyclyl, and —NR²⁰⁷R²⁰⁸;
R²⁰⁷ is H or alkyl;
R²⁰⁸ is selected from: H, alkyl, alkylcarbonyl, (cycloalkyl)alkylcarbonyl, aralkylcarbonyl, arylcarbonyl, (heterocyclyl)carbonyl, and aralkyl;
W² is selected from aryl and heteroaryl;
R^{204a} and R^{204b} are independently selected from: H, alkyl, cycloalkyl, and haloalkyl;
R²⁰⁵ and R²⁰⁶ are independently selected from: H, OH, halogen, CN, NO₂, alkyl, haloalkyl, haloalkoxy, aryl, heterocyclyl, cycloalkyl, —NR^{204a}R^{204b}, —CONR^{204a}R^{204b}, —OR^{204a}R^{204b}, —NR^{204a}COR^{204b}, —SO₂NR^{204a}R^{204b}, and —NR^{204a}SO₂R^{204b};
preferably, in general formula II, the E3L moiety has the following structure: 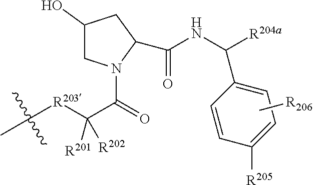
wherein R²⁰³′ is selected from: —NHCO— and 5- to 6-membered heteroarylene, such as 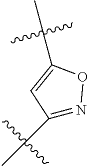 [SMILES: *c1cc(*)on1]
and 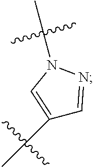 [SMILES: *c1cnn(*)c1]
or the E3L moiety has the following structure: 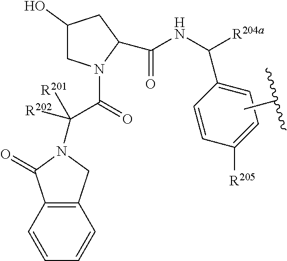
more preferably, the E3L moiety has the following structure: 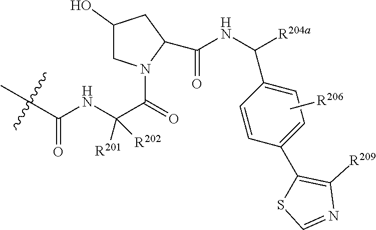
more preferably, the E3L moiety is selected from the following structures:

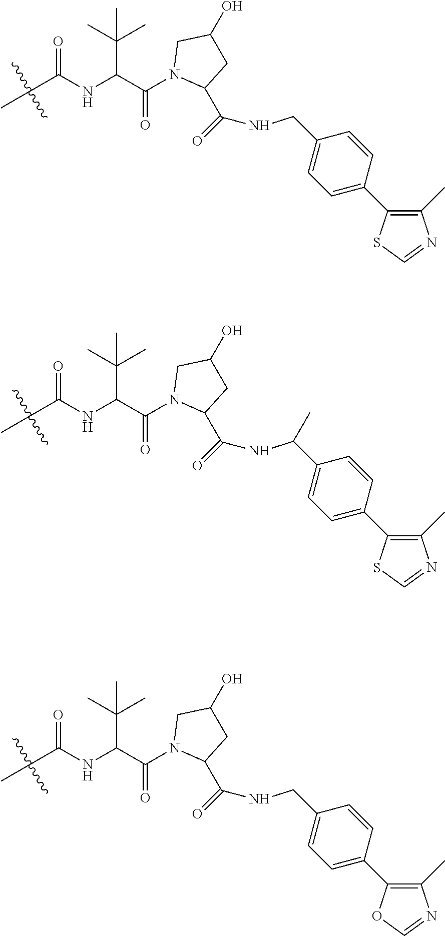 [SMILES: *C(=O)NC(C(=O)N1CC(O)CC1C(=O)NCc1ccc(-c2scnc2C)cc1)C(C)(C)C ; *C(=O)NC(C(=O)N1CC(O)CC1C(=O)NC(C)c1ccc(-c2scnc2C)cc1)C(C)(C)C ; *C(=O)NC(C(=O)N1CC(O)CC1C(=O)NCc1ccc(-c2ocnc2C)cc1)C(C)(C)C]

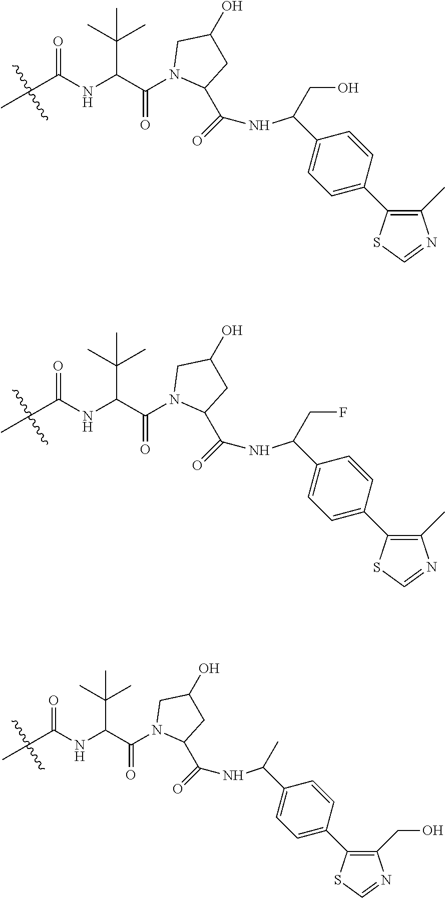 [SMILES: *C(=O)NC(C(=O)N1CC(O)CC1C(=O)NC(CO)c1ccc(-c2scnc2C)cc1)C(C)(C)C ; *C(=O)NC(C(=O)N1CC(O)CC1C(=O)NC(CF)c1ccc(-c2scnc2C)cc1)C(C)(C)C ; *C(=O)NC(C(=O)N1CC(O)CC1C(=O)NC(C)c1ccc(-c2scnc2CO)cc1)C(C)(C)C]

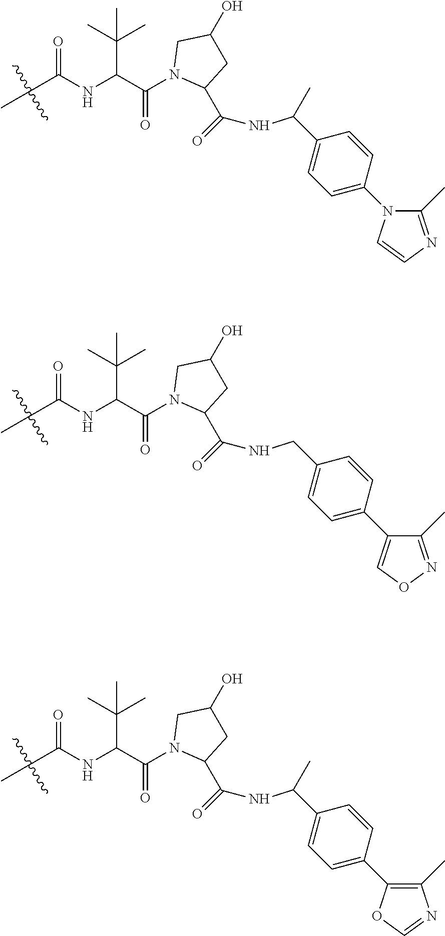 [SMILES: *C(=O)NC(C(=O)N1CC(O)CC1C(=O)NC(C)c1ccc(-n2ccnc2C)cc1)C(C)(C)C ; *C(=O)NC(C(=O)N1CC(O)CC1C(=O)NCc1ccc(-c2conc2C)cc1)C(C)(C)C ; *C(=O)NC(C(=O)N1CC(O)CC1C(=O)NC(C)c1ccc(-c2ocnc2C)cc1)C(C)(C)C]

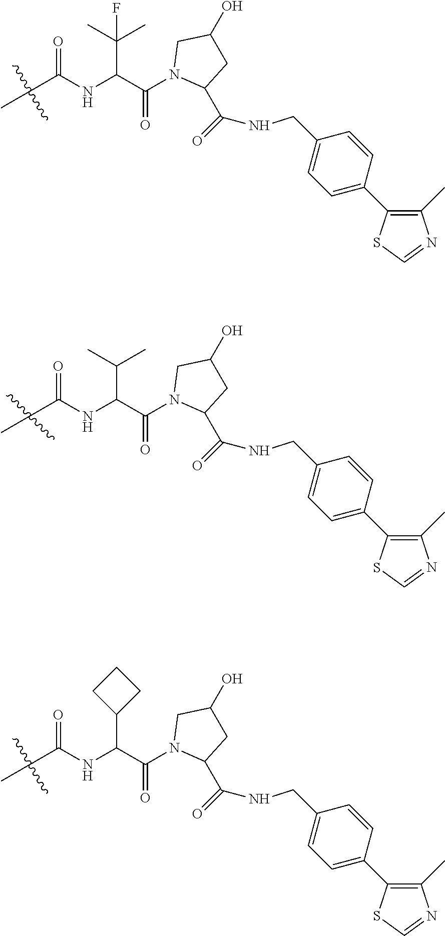 [SMILES: *C(=O)NC(C(=O)N1CC(O)CC1C(=O)NCc1ccc(-c2scnc2C)cc1)C(C)(C)F ; *C(=O)NC(C(=O)N1CC(O)CC1C(=O)NCc1ccc(-c2scnc2C)cc1)C(C)C ; *C(=O)NC(C(=O)N1CC(O)CC1C(=O)NCc1ccc(-c2scnc2C)cc1)C1CCC1]

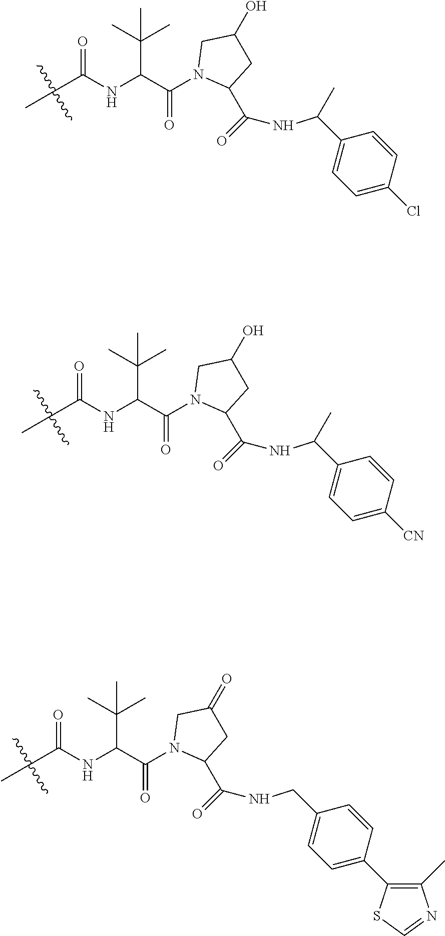 [SMILES: *C(=O)NC(C(=O)N1CC(O)CC1C(=O)NC(C)c1ccc(Cl)cc1)C(C)(C)C ; *C(=O)NC(C(=O)N1CC(O)CC1C(=O)NC(C)c1ccc(C#N)cc1)C(C)(C)C ; *C(=O)NC(C(=O)N1CC(=O)CC1C(=O)NCc1ccc(-c2scnc2C)cc1)C(C)(C)C]

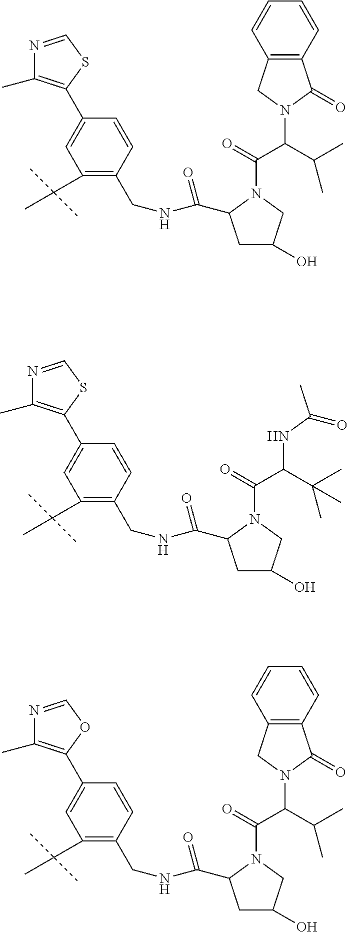

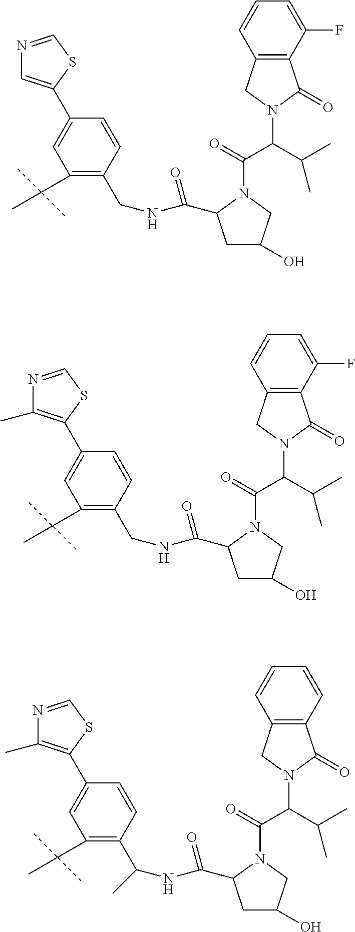

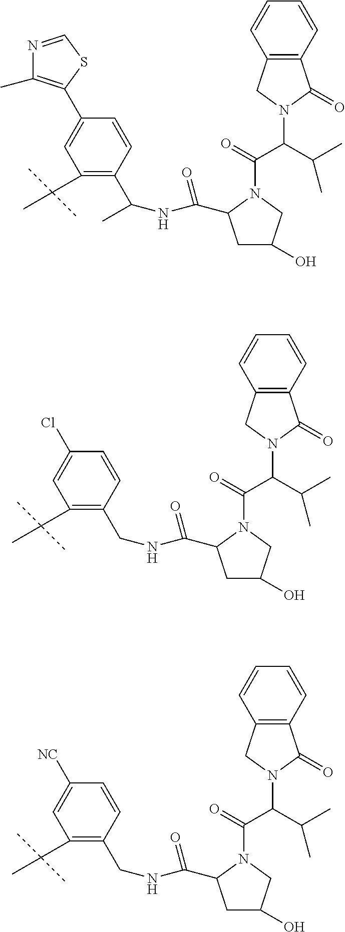

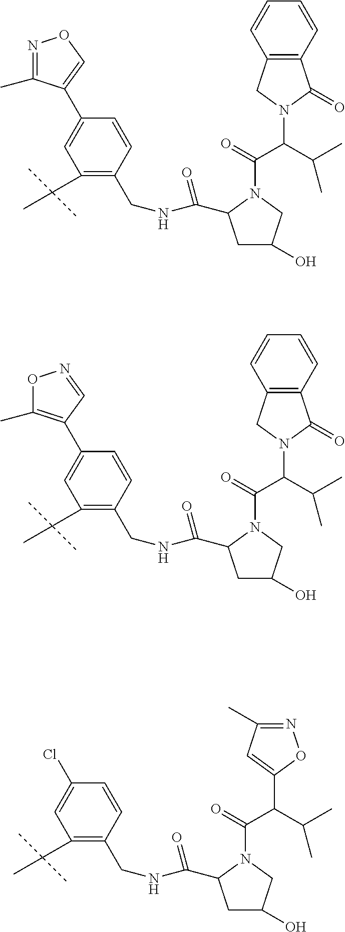

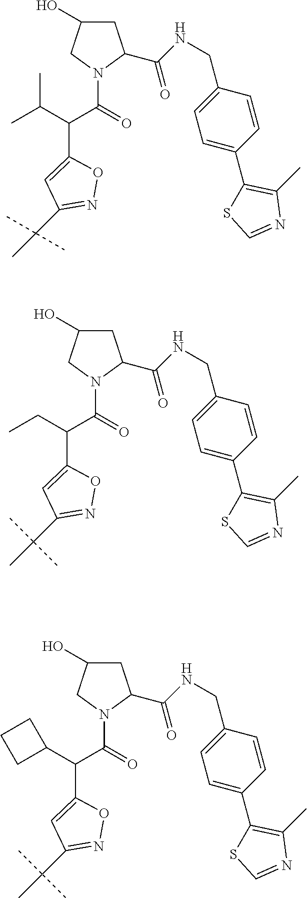

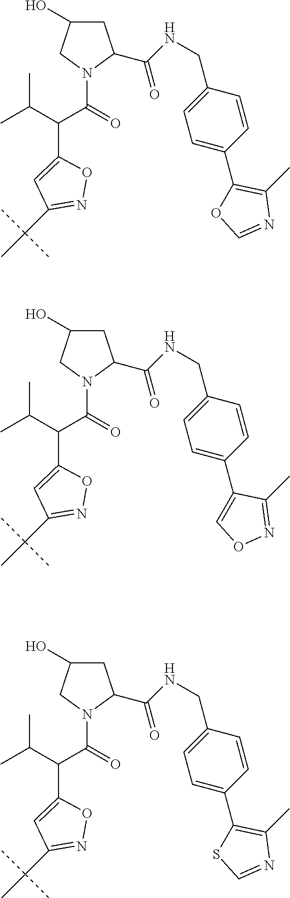

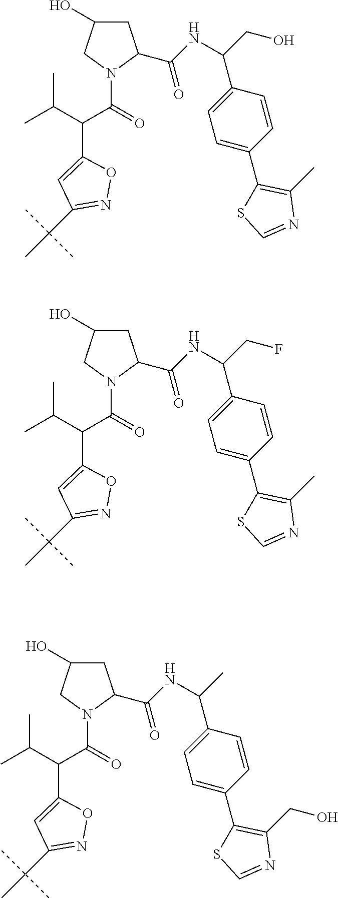

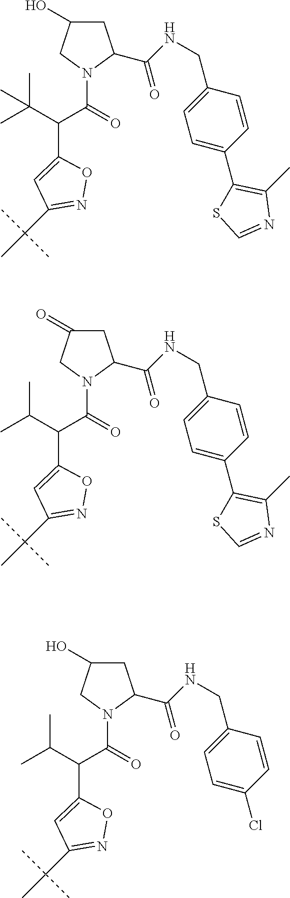

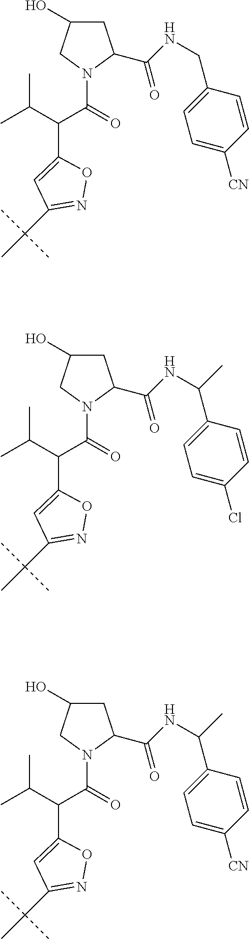

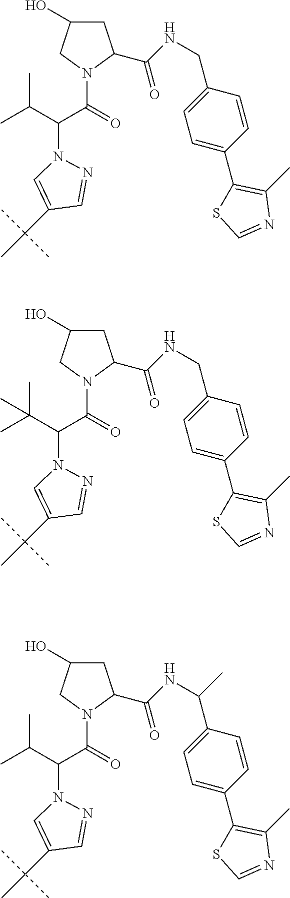

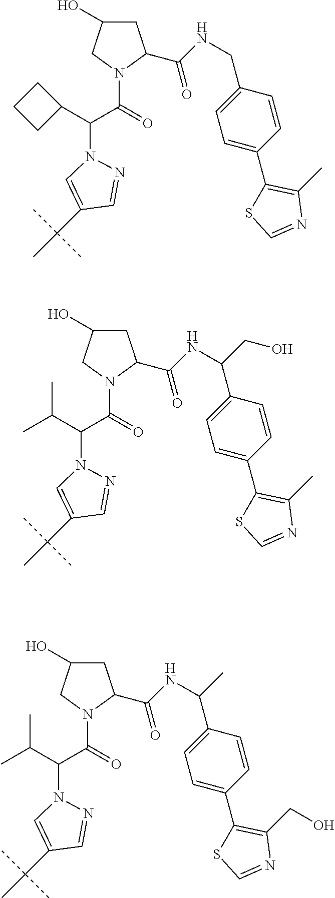

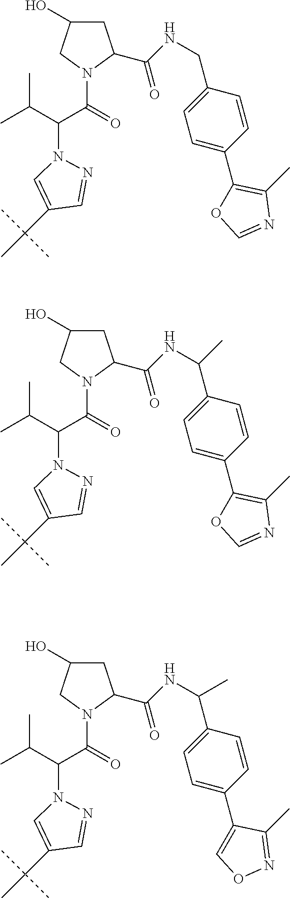

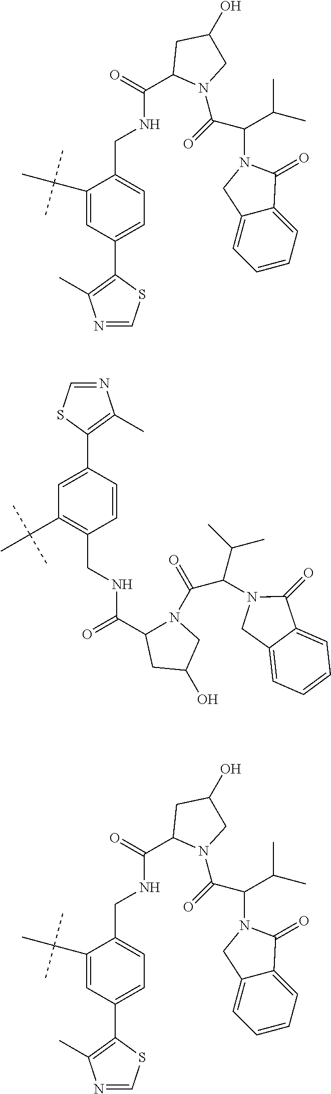

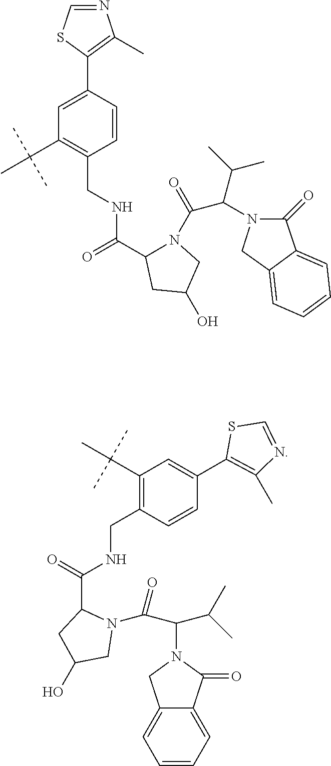

47. The targeted protein degraders according to claim 44, wherein L₃ is —C(O)—; and/or L₁ is O or a single bond.

48. The targeted protein degrader according to claim 44, wherein the targeted protein degrader represented by general formula II has the following structure: 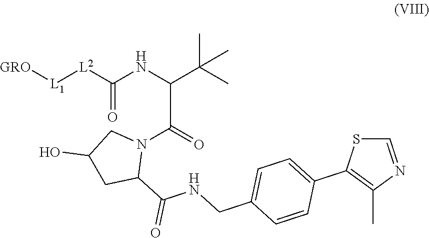
preferably, the targeted protein degrader represented by general formula II has the following structure: 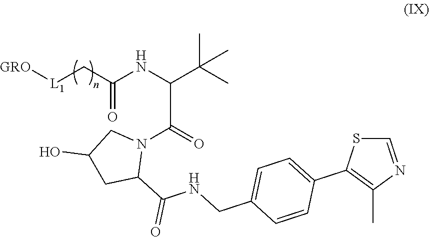
wherein n is an integer of 1-10;
preferably, the targeted protein degrader represented by general formula II has the following structure:

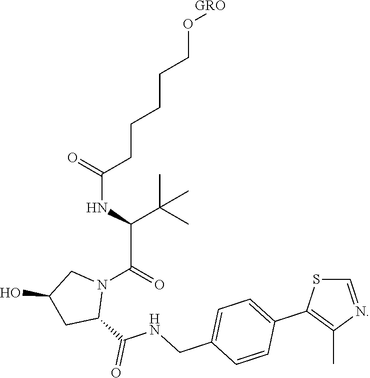

49. A pharmaceutical composition, comprising the targeted protein degrader according to claim 28, and one or more pharmaceutically acceptable auxiliary materials.

50. A delivery system for a targeted protein degrader, comprising the targeted protein degrader according to claim 28 and a carrier, wherein
preferably, the carrier is a viral carrier or a non-viral carrier;
more preferably, the viral carrier is a lentivirus, an adenovirus, or an adeno-associated viral carrier;
more preferably, the non-viral carrier is a lipid nanoparticle (LNP), a polymeric nanocarrier, an inorganic nanocarrier, a protein carrier, or an exosome.

51. Use of the targeted protein degrader according to claim 28, the pharmaceutical composition, and the delivery system for the targeted protein degrader in the preparation of a medicament for preventing and/or treating a disease, wherein
preferably, the disease is a disease that can be prevented and/or treated beneficially by inhibiting/degrading the target protein, and is preferably selected from: at least one of a tumor, an autoimmune disease, an inflammatory disease, a disease associated with pathogen infection, a neurodegenerative disease, a cardiovascular disease, a metabolic disease, a fibrotic disease, and an ophthalmic disease.

52. Use of a GRO as a recruitment element of MDM2 in the preparation of a medicament, wherein the GRO is a guanine-rich oligonucleotide capable of specifically binding to NCL, and preferably, the GRO has stable G4 structural characteristics.

53. The use according to claim 52, wherein the GRO is one of DNA aptamer AS1411 and an AS1411 derivative/analog; preferably, the AS1411 derivative/analog is one of GRO29A, GRO15A, AT11, AT11-L0, AT21, AT27, GRO29-2, GRO29-3, GRO29-5, GRO29-13, GRO14C, GRO25A, GRO28A, GRO28B, GRO29-6, GRO32A, GRO32B, GRO56A, GRO A, GRO B, GRO C, GRO D, GRO E, GRO F, GRO G, GRO H, GRO I, GRO J, GRO K, GRO L, and GRO M;
preferably, the GRO comprises a chemical modification, a nucleic acid unit replacement, or a linkage to a functional group on the GRO, wherein
the chemical modification is that at least one base is modified, and the chemical modification comprises at least one of phosphorylation, methylation, amination, sulfydrylation, isotopication, phosphorothioate backbone modification, methoxy modification, and fluoro modification;
the nucleic acid unit replacement is that at least one nucleic acid unit is replaced by LNA, UNA, or GNA;
the functional group comprises at least one of a fluorophore, a radioactive group, a therapeutic drug, biotin, digoxigenin, a nano-luminescent material, a nucleic acid substance, or an enzyme label.

54. The use according to claim 52, wherein the medicament is a targeted protein degrader and is preferably selected from: a PROTAC, a molecular glue, a CHAMP, an LYTAC, a GlucTAC, an AbTAC, an AUTAC, an AUTOTAC, an ATTEC, an antibody-PROTAC conjugate, and an MADTAC, and more preferably, the medicament is a PROTAC.


SEQUENCE LISTING (47 sequences)

SEQ ID NO: 1 - DNA, 26 bp, synthetic construct
  source 1..26: mol_type = other DNA
ggtggtggtggttgtggtggtggtgg

SEQ ID NO: 2 - DNA, 29 bp, synthetic construct
  source 1..29: mol_type = other DNA
tttggtggtggtggttgtggtggtggtgg

SEQ ID NO: 3 - DNA, 15 bp, synthetic construct
  source 1..15: mol_type = other DNA
gttgtttggggtggt

SEQ ID NO: 4 - DNA, 28 bp, synthetic construct
  source 1..28: mol_type = other DNA
tggtggtggttgttgtggtggtggtggt

SEQ ID NO: 5 - DNA, 11 bp, synthetic construct
  source 1..11: mol_type = other DNA
cttctgggaaa

SEQ ID NO: 6 - DNA, 11 bp, synthetic construct
  source 1..11: mol_type = other DNA
tttcccagaag

SEQ ID NO: 7 - DNA, 11 bp, synthetic construct
  source 1..11: mol_type = other DNA
gagcacgtggt

SEQ ID NO: 8 - DNA, 11 bp, synthetic construct
  source 1..11: mol_type = other DNA
accacgtgctc

SEQ ID NO: 9 - RNA, 25 nt, synthetic construct
  source 1..25: mol_type = other RNA
attagcgcattttaacatagggtgc

SEQ ID NO: 10 - RNA, 23 nt, synthetic construct
  source 1..23: mol_type = other RNA
ccttttgtttttccctctccagg

SEQ ID NO: 11 - RNA, 20 nt, synthetic construct
  source 1..20: mol_type = other RNA
tatttttccctctccaccct

SEQ ID NO: 12 - DNA, 25 bp, synthetic construct
  source 1..25: mol_type = other DNA
tgtgggggtggacgggccgggtaga

SEQ ID NO: 13 - DNA, 26 bp, synthetic construct
  source 1..26: mol_type = other DNA
cctcctcctccttctcctcctcctcc

SEQ ID NO: 14 - DNA, 18 bp, synthetic construct
  source 1..18: mol_type = other DNA
agattagggtgagggtga

SEQ ID NO: 15 - DNA, 35 bp, synthetic construct
  source 1..35: mol_type = other DNA
ggaattcccggtgcgccgatcgccggatataactt

SEQ ID NO: 16 - RNA, 21 nt, synthetic construct
  source 1..21: mol_type = other RNA
  modified_base 20..21: mod_base = OTHER; note = thymine
ttctccgaacgtgtcacgttt

SEQ ID NO: 17 - RNA, 23 nt, synthetic construct
  source 1..23: mol_type = other RNA
  modified_base 22..23: mod_base = OTHER; note = thymine
ggatgacgacgacgacgaagatt

SEQ ID NO: 18 - RNA, 21 nt, synthetic construct
  source 1..21: mol_type = other RNA
  modified_base 20..21: mod_base = OTHER; note = thymine
gcttggcctacagtcatcttt

SEQ ID NO: 19 - DNA, 50 bp, synthetic construct
  source 1..50: mol_type = other DNA
atcaggctggatggtagctcggtcggggtgggtgggttggcaagtctgat

SEQ ID NO: 20 - DNA, 26 bp, synthetic construct
  source 1..26: mol_type = other DNA
tggtggtggttgttgggtggtggggt

SEQ ID NO: 21 - DNA, 28 bp, synthetic construct
  source 1..28: mol_type = other DNA
tggtggtggtggttgtggtgttggtggt

SEQ ID NO: 22 - DNA, 28 bp, synthetic construct
  source 1..28: mol_type = other DNA
tggtggtggtggttgtggtggtggtgtt

SEQ ID NO: 23 - DNA, 29 bp, synthetic construct
  source 1..29: mol_type = other DNA
tttggtggtggtggttttggtggtggtgg

SEQ ID NO: 24 - DNA, 29 bp, synthetic construct
  source 1..29: mol_type = other DNA
tttggtggtggtggtggtggtggtggtgg

SEQ ID NO: 25 - DNA, 29 bp, synthetic construct
  source 1..29: mol_type = other DNA
tttggtggtggtggtttgggtggtggtgg

SEQ ID NO: 26 - DNA, 13 bp, synthetic construct
  source 1..13: mol_type = other DNA
tggtggtggtggt

SEQ ID NO: 27 - DNA, 14 bp, synthetic construct
  source 1..14: mol_type = other DNA
ggtggttgtggtgg

SEQ ID NO: 28 - DNA, 25 bp, synthetic construct
  source 1..25: mol_type = other DNA
ggttggggtgggtggggtgggtggg

SEQ ID NO: 29 - DNA, 28 bp, synthetic construct
  source 1..28: mol_type = other DNA
tttggtggtggtggttgtggtggtggtg

SEQ ID NO: 30 - DNA, 28 bp, synthetic construct
  source 1..28: mol_type = other DNA
tttggtggtggtggtgtggtggtggtgg

SEQ ID NO: 31 - DNA, 29 bp, synthetic construct
  source 1..29: mol_type = other DNA
ggtggtggtggttgtggtggtggtggttt

SEQ ID NO: 32 - DNA, 32 bp, synthetic construct
  source 1..32: mol_type = other DNA
ggtggttgtggtggttgtggtggttgtggtgg

SEQ ID NO: 33 - DNA, 32 bp, synthetic construct
  source 1..32: mol_type = other DNA
tttggtggtggtggttgtggtggtggtggttt

SEQ ID NO: 34 - DNA, 56 bp, synthetic construct
  source 1..56: mol_type = other DNA
ggtggtggtggttgtggtggtggtggttgtggtggtggtggttgtggtggtggtgg

SEQ ID NO: 35 - DNA, 24 bp, synthetic construct
  source 1..24: mol_type = other DNA
ttagggttagggttagggttaggg

SEQ ID NO: 36 - DNA, 11 bp, synthetic construct
  source 1..11: mol_type = other DNA
ggtggtggtgg

SEQ ID NO: 37 - DNA, 14 bp, synthetic construct
  source 1..14: mol_type = other DNA
ggtggttgtggtgg

SEQ ID NO: 38 - DNA, 15 bp, synthetic construct
  source 1..15: mol_type = other DNA
ggttggtgtggttgg

SEQ ID NO: 39 - DNA, 10 bp, synthetic construct
  source 1..10: mol_type = other DNA
gggttttggg

SEQ ID NO: 40 - DNA, 20 bp, synthetic construct
  source 1..20: mol_type = other DNA
ggttttggttttggttttgg

SEQ ID NO: 41 - DNA, 15 bp, synthetic construct
  source 1..15: mol_type = other DNA
ggttggtgtggttgg

SEQ ID NO: 42 - DNA, 12 bp, synthetic construct
  source 1..12: mol_type = other DNA
ggggttttgggg

SEQ ID NO: 43 - DNA, 10 bp, synthetic construct
  source 1..10: mol_type = other DNA
gggttttggg

SEQ ID NO: 44 - DNA, 28 bp, synthetic construct
  source 1..28: mol_type = other DNA
ggggttttggggttttggggttttgggg

SEQ ID NO: 45 - DNA, 24 bp, synthetic construct
  source 1..24: mol_type = other DNA
ttggggttggggttggggttgggg

SEQ ID NO: 46 - DNA, 16 bp, synthetic construct
  source 1..16: mol_type = other DNA
gggtgggtgggtgggt

SEQ ID NO: 47 - DNA, 26 bp, synthetic construct
  source 1..26: mol_type = other DNA
ggttttggttttggttttggttttgg